(19) United States
(12) Patent Application Publication
(10) Pub. No.: US 2025/0123280 A1
(43) Pub. Date: Apr. 17, 2025

(54) LABEL-FREE DETECTION OF PROTEASE ACTIVITY

(71) Applicant: OREGON HEALTH & SCIENCE UNIVERSITY, Portland, OR (US)

(72) Inventors: Adem Yildirim, Portland, OR (US); Bruce Branchaud, Portland, OR (US); Justin Plaut, Portland, OR (US); Srivathsan Ranganathan, Portland, OR (US); Sean Speese, Portland, OR (US); Corey Dambacher, Portland, OR (US); Sarah Barnhill, Portland, OR (US); Emma Olson, Portland, OR (US)

(21) Appl. No.: 18/290,740

(22) PCT Filed: Jul. 20, 2022
(86) PCT No.: PCT/US2022/037769

§ 371 Date: Jan. 19, 2024

Related U.S. Application Data
(60) Provisional application No. 63/224,309, filed on Jul. 21, 2021.
(60) Provisional application No. 63/223,907, filed on Jul. 20, 2021.

(51) Int. Cl. G01N 33/574; C07K 7/06; C07K 7/08; C12Q 1/37; G01N 33/58

(52) U.S. Cl. CPC G01N 33/57407; C07K 7/06; C07K 7/08; C12Q 1/37; G01N 33/582; G01N 2800/7028

(57) ABSTRACT

The present disclosure provides self-assembling polypeptides and methods for detecting protease activity by enzyme-instructed beta-sheet formation. A self-assembling polypeptide comprises a β-strand motif configured to self-assemble with one or more nominally identical β-strand motifs and form an anti-parallel beta-sheet structure. The β-strand motif being operatively connected to a hydrophilic motif by a protease substrate motif that comprises a protease cleavage site configured to specifically hybridize with a protease. Whereby, when in an aqueous milieu and upon hybridization of the protease to the protease cleavage site, the protease cleaves the self-assembling polypeptide and dissociates the β-strand motif allowing the dissociated β-strand motif to self-assemble with the one or more nominally identical β-strand motifs and thereby form the anti-parallel β-sheet structure. A β-sheet intercalating dye is complexed with the anti-parallel β-sheet structure and detection of fluorescent signal indicates proteolytic activity.

### COPYRIGHT NOTICE

© 2022 Oregon Health & Science University. A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR § 1.71(d).

### CROSS-REFERENCE TO RELATED APPLICATIONS

The present application claims the benefit of U.S. Provisional Patent Application No. 63/223,907, filed Jul. 20, 2021, and U.S. Provisional Patent Application No. 63/224,309, filed Jul. 21, 2021, which are hereby incorporated by reference in their entirety.

### TECHNICAL FIELD

This disclosure relates generally to the field of biotechnology and in particular to utilizing enzyme-instructed self-assembly (EISA) and related products and uses thereof.

### BACKGROUND

Over the past few decades, various assays have been developed to detect protease activity, with the most widely reported ones being quenched probes. In quenched probe detection scheme, a fluorophore is attached to a protease substrate where its emission is typically quenched by internal energy transfer to the peptide substrate, or a quencher molecule or a nanoparticle. These probes often suffer from high background signal due to the incomplete quenching of the dyes and, thus, low signal enhancement after protease cleavage. Incorporating self-assembly motifs to conventional quenched probes can lower their background signal by further quenching fluorophore emission through aggregation-induced quenching. The utilization of peptide self-assembly offers opportunities to design molecular probes for more sensitive detection of protease activity. However, previously developed EISA or quenching-based protease activity assays often require labeling the protease substrates with a fluorophore or other type of molecular probe, which complicates their synthesis and increases cost.

Thus, the development of label-free EISA methods detection of protease activity would lower background signal, increase sensitivity, simplify probe synthesis, reduce cost.

### SUMMARY OF THE DISCLOSURE

The disclosed materials and methods relate to detecting protease activity. The present disclosure provides compositions and methods for detecting protease activity by enzyme-instructed beta-sheet formation. In an exemplary embodiment, a self-assembling polypeptide comprises a β-strand motif configured to self-assemble with one or more nominally identical β-strand motifs and form an anti-parallel beta-sheet structure. The β-strand motif is operatively connected to a hydrophilic motif by a protease substrate motif and the protease substrate motif comprises a protease cleavage site configured to specifically hybridize with a protease. Whereby, when in an aqueous milieu and upon hybridization of the protease to the protease cleavage site, the protease cleaves the self-assembling polypeptide and dissociates the β-strand motif allowing the dissociated β-strand motif to self-assemble with the one or more nominally identical β-strand motifs and thereby form the anti-parallel β-sheet structure.

In some aspects, the disclosure provides a method for detecting proteolytic cleavage by enzyme-instructed β-sheet formation. The method comprises administering, into an aqueous milieu, a set of one or more self-assembling polypeptides A β-sheet intercalating dye configured to emit a fluorescent signal is administered into the aqueous milieu and forms a complex with one or more anti-parallel β-sheet structures formed by the self-assembly of β-strand motifs. The fluorescent signal is then detected to thereby indicate the presence of the protease in the aqueous milieu.

Additional aspects and advantages will be apparent from the following detailed description of preferred embodiments, which proceeds with reference to the accompanying drawings.

### BRIEF DESCRIPTION OF THE DRAWINGS

FIGS. 1A, 1B, and 1C are schematic representations of label-free protease detection using enzyme-instructed β-sheet structure formation.

FIG. 2 is a graph of fluorescence spectra of ThT in the presence or absence of peptide 2 in assay buffer.

FIGS. 3A and 3B show TEM images of self-assembled structures of peptide 2.

FIGS. 4A and 4B show AFM images of self-assembled structures of peptide 2.

FIGS. 5A and 5B are line and percent graphs showing CD spectrum of peptide 2 suspended in assay buffer.

FIGS. 6A and 6B show TEM images of peptide 1 incubated with legumain after bath sonication.

FIGS. 7A and 7B show AFM characterization of peptide 1 incubated with legumain.

FIG. 8 shows CD spectra of peptide 1 before and after legumain addition.

FIGS. 9A and 9B are line graphs showing, respectively, fluorescent intensity enhancement of ThT at different concentrations and absorbance spectra of ThT in the present or absence of peptide 1.

FIGS. 10 and 11 are line graphs showing, respectively, the kinetics of fluorescence signal change with or without legumain and the percent inhibition of the legumain activity at different inhibitor concentrations.

FIG. 12A is a line graph showing the representative fluorescence spectra of ThT in the presence of different peptide 1 amounts; and FIG. 12B is a line graph showing the relative fluorescence intensity of ThT in the presence of different amounts of peptide 1.

FIG. 13 is a FTIR spectra of peptide 1, before and after incubation with legumain and peptide 2.

FIG. 14 is a fluorescence spectra of peptide 2 in DMSO or buffer.

FIGS. 15 and 16 shows various stick models of peptide 2.

FIGS. 17A and 17B are, respectively, liquid chromatography (LC) and mass spectrometry (MS) data of peptide 1 before and after incubating with legumain.

FIG. 18 shows parallel photographs of peptide 1 solutions incubated with or without legumain after centrifugation showing ThT-complexed self-assembled beta-sheet structures; FIG. 19 shows CD spectrum of peptide 1 after incubation for two weeks with legumain; and FIG. 20 is a graph showing the fluorescence intensity enhancement of ThT at different legumain concentrations.

FIG. 21 shows representative absorbance spectra of ThT in the presence of peptide 1 after a two hour incubation with different amounts of legumain.

FIG. 22A is a line graph showing representative fluorescence spectra of ThT in the presence or absence of peptide 1 after incubation with legumain; and, FIG. 22B is a bar graph showing the fluorescence intensity enhancement of ThT at different legumain concentrations with or without legumain.

FIG. 23 is a bar graph showing the relative fluorescence intensity of ThT in peptide 1 solution with or without legumain at different time points.

FIGS. 24A and 24B are representative fluorescence spectra of ThT in, respectively, 20% plasma and albumin depleted 20% plasma, in the presence or absence of peptide 1 after incubation with or without legumain.

FIG. 25 is a fluorescence spectra of peptide 1 samples incubated in 10% plasma in the presence or absence of Legumain after separating the peptide aggregates.

FIGS. 26A and 26B are line graphs showing the fluorescence of ThT at different concentrations in 10% plasma, respectively, without peptide1 or legumain and with peptide 1 and legumain.

FIG. 27 is a line graph of a Z-AAN-AMC probe after incubation with different amounts of legumain in buffer or 10% plasma; and FIG. 28 is a line graph of representative fluorescence spectra of MCAAD-3 in the presence or absence of peptide 1 with different amounts of legumain.

FIG. 28 is a line graph of representative fluorescence spectra of MCAAD-3 in the presence or absence of peptide 1 with different amounts of legumain.

FIG. 29A shows the representative fluorescence spectra of ThT after treating peptide 3 with 1000 ng/mL and 0 ng/mL cathepsin B. FIG. 29B shows the fold-increase in ThT fluorescence intensity after treatment of peptide 3 with 1000 ng/mL as compared to untreated peptide 3 (0 ng/mL cathepsin B).

### SEQUENCE LISTING

Any nucleic acid and amino acid sequences listed herein or in the accompanying sequence listing are shown using standard abbreviations for nucleotide bases and amino acids, as defined in 37 C.F.R. § 1.822. In as least some cases, only one strand of each nucleic acid sequence is shown, but the complementary strand is understood as included by any reference to the displayed strand.

SEQ ID NO: 1 is an amino acid sequence of an exemplary ß-strand motif, consisting of the amino acid sequence: Fmoc-Phe-Lys-Phe-Glu, in which the N-terminus is modified to comprise a Fmoc protecting group.

SEQ ID NO: 2 is an amino acid sequence of an exemplary ß-strand motif consisting of the amino acid sequence: Fmoc-Phe-Phe, in which the N-terminus is modified to comprise a Fmoc protecting group.

SEQ ID NO: 3 is an amino acid sequence of an exemplary ß-strand motif consisting of the amino acid sequence: Fmoc-Phe-Phe-(D-Lys)-(D-Lys), in which the N-terminus is modified to comprise a Fmoc protecting group.

SEQ ID NO: 4 is an amino acid sequence of an exemplary ß-strand motif consisting of the amino acid sequence: Fmoc-Phe-(D-Lys)-Phe-(D-Lys),in which the N-terminus is modified to comprise a Fmoc protecting group.

SEQ ID NO: 5 is an amino acid sequence of an exemplary ß-strand motif consisting of the amino acid sequence: Phe-Glu-Phe-Glu-Phe-Lys-Phe-Lys.

SEQ ID NO: 6 is an amino acid sequence of an exemplary ß-strand motif consisting of the amino acid sequence: Phe-Glu-Phe-Lys-Phe-Glu-Phe-Lys.

SEQ ID NO: 7 is an amino acid sequence of an exemplary ß-strand motif consisting of the amino acid sequence: Phe-Lys-Phe-Glu-Phe-Lys-Phe-Glu.

SEQ ID NO: 8 is an amino acid sequence of an exemplary ß-strand motif consisting of the amino acid sequence: (D-Phe)-(D-Lys)-(D-Phe)-(D-Glu)-(D-Phe)-(D-Lys)-(D-Phe)-(D-Glu).

SEQ ID NO: 9 is an amino acid sequence of an exemplary ß-strand motif consisting of the amino acid sequence: Acetyl-Phe-Lys-Phe-Glu-Phe-Lys-Phe-Glu-Amide, in which the C-terminus and N-terminus are modified to comprise, respectively, an acetyl protecting group and an amide protecting group.

SEQ ID NO: 10 is an amino acid sequence of an exemplary ß-strand motif consisting of the amino acid sequence: Acetyl-Phe-Lys-Phe-Glu-Phe-Lys-Phe-Amide, in which the C-terminus and N-terminus are modified to comprise, respectively, an acetyl protecting group and an amide protecting group.

SEQ ID NO: 11 is an amino acid sequence of an exemplary hydrophilic motif consisting of the amino acid sequence: Glu-Glu-Glu-Gly-Ser-Gly-Glu-Glu-Glu.

SEQ ID NO: 12 is an amino acid sequence of an exemplary hydrophilic motif consisting of the amino acid sequence: Asp-Asp-Asp-Gly-Ser-Gly-Glu-Glu-Glu.

SEQ ID NO: 13 is an amino acid sequence of an exemplary hydrophilic motif consisting of the amino acid sequence: Glu-Glu-Glu-Gly-Ser-Gly-Asp-Asp-Asp.

SEQ ID NO: 14 is an amino acid sequence of an exemplary hydrophilic motif consisting of the amino acid sequence: Glu-Glu-Glu-Gly-Ser-Gly-Glu-Glu-Glu-Gly-Ser-Gly-Glu-Glu-Glu.

SEQ ID NO: 15 is an amino acid sequence of an exemplary hydrophilic motif consisting of the amino acid sequence: Asp-Asp-Asp-Gly-Ser-Gly-Glu-Glu-Glu-Gly-Ser-Gly-Glu-Glu-Glu.

SEQ ID NO: 16 is an amino acid sequence of an exemplary hydrophilic motif consisting of the amino acid sequence: Glu-Glu-Glu-Gly-Ser-Gly-Asp-Asp-Asp-Gly-Ser-Gly-Glu-Glu-Glu.

SEQ ID NO: 17 is an amino acid sequence of an exemplary hydrophilic motif consisting of the amino acid sequence: Asp-Asp-Asp-Gly-Ser-Gly-Asp-Asp-Asp-Gly-Ser-Gly-Asp-Asp-Asp.

SEQ ID NO: 18 is an amino acid sequence of an exemplary hydrophilic motif consisting of the amino acid sequence: Asp-Asp-Gly-Asp-Asp.

SEQ ID NO: 19 is an amino acid sequence of an exemplary hydrophilic motif consisting of the amino acid sequence: Asp-Asp-Gly-Asp-Asp-Gly-Asp-Asp.

SEQ ID NO: 20 is an amino acid sequence of an exemplary hydrophilic motif consisting of the amino acid sequence: Asp-Asp-Gly-Asp-Asp-Gly-Asp-Asp-Gly-Asp-Asp.

SEQ ID NO: 21 is an amino acid sequence of an exemplary hydrophilic motif consisting of the amino acid sequence: Asp-Asp-Gly-Asp-Asp-Gly-Asp-Asp-Gly-Asp-Asp-Gly-Asp-Asp.

SEQ ID NO: 22 is an amino acid sequence of an exemplary hydrophilic motif consisting of the amino acid sequence: Glu-Glu-Gly-Glu-Glu.

SEQ ID NO: 23 is an amino acid sequence of an exemplary hydrophilic motif consisting of the amino acid sequence: Glu-Glu-Gly-Glu-Glu-Gly-Glu-Glu.

SEQ ID NO: 24 is an amino acid sequence of an exemplary hydrophilic motif consisting of the amino acid sequence: Glu-Glu-Gly-Glu-Glu-Gly-Glu-Glu-Gly-Glu-Glu.

SEQ ID NO: 25 is an amino acid sequence of an exemplary hydrophilic motif consisting of the amino acid sequence: Glu-Glu-Gly-Glu-Glu-Gly-Glu-Glu-Gly-Glu-Glu-Gly-Glu-Glu.

SEQ ID NO: 26 is an amino acid sequence of an exemplary hydrophilic motif consisting of the amino acid sequence: Glu-Glu-Gly-Lys-Asp-Asp-Gly-Glu-Glu-Gly-Asp-Asp.

SEQ ID NO: 27 is an amino acid sequence of an exemplary hydrophilic motif consisting of the amino acid sequence: Asp-Asp-Gly-Glu-Glu-Gly-Asp-Asp-Gly-Glu-Glu.

SEQ ID NO: 28 is an amino acid sequence of an exemplary hydrophilic motif consisting of the amino acid sequence: Glu-Glu-Gly-Lys-Lys-Gly-Glu-Glu-Gly-Lys-Lys-Gly-Glu-Glu.

SEQ ID NO: 29 is an amino acid sequence of an exemplary hydrophilic motif consisting of the amino acid sequence: Asp-Asp-Gly-Glu-Glu-Gly-Lys-Lys-Gly-Glu-Glu-Gly-Lys-Lys.

SEQ ID NO: 30 is an amino acid sequence of an exemplary hydrophilic motif consisting of the amino acid sequence: Asp-Ser-Asp-Ser (SEQ ID NO: 30).

SEQ ID NO: 31 is an amino acid sequence of an exemplary hydrophilic motif consisting of the amino acid sequence: Asp-Ser-Asp-Ser-Asp-Ser (SEQ ID NO: 31).

SEQ ID NO: 32 is an amino acid sequence of an exemplary hydrophilic motif consisting of the amino acid sequence: Asp-Ser-Asp-Ser-Asp-Ser-Asp-Ser.

SEQ ID NO: 33 is an amino acid sequence of an exemplary hydrophilic motif consisting of the amino acid sequence: Asp-Ser-Asp-Ser-Asp-Ser-Asp-Ser-Asp-Ser.

SEQ ID NO: 34 is an amino acid sequence of an exemplary hydrophilic motif consisting of the amino acid sequence: Asp-Ser-Asp-Ser-Asp-Ser-Asp-Ser-Asp-Ser-Asp-Ser.

SEQ ID NO: 35 is an amino acid sequence of an exemplary hydrophilic motif consisting of the amino acid sequence: Asp-Ser-Asp-Ser-Asp-Ser-Asp-Ser-Asp-Ser-Asp-Ser-Asp-Ser.

SEQ ID NO: 36 is an amino acid sequence of an exemplary hydrophilic motif consisting of the amino acid sequence: Asp-Ser-Asp-Ser-Asp-Ser-Asp-Ser-Asp-Ser-Asp-Ser-Asp-Ser-Asp-Ser.

SEQ ID NO: 37 is an amino acid sequence of an exemplary hydrophilic motif consisting of the amino acid sequence: Glu-Ser-Glu-Ser.

SEQ ID NO: 38 is an amino acid sequence of an exemplary hydrophilic motif consisting of the amino acid sequence: Glu-Ser-Glu-Ser-Glu-Ser.

SEQ ID NO: 39 is an amino acid sequence of an exemplary hydrophilic motif consisting of the amino acid sequence: Glu-Ser-Glu-Ser-Glu-Ser-Glu-Ser.

SEQ ID NO: 40 is an amino acid sequence of an exemplary hydrophilic motif consisting of the amino acid sequence: Glu-Ser-Glu-Ser-Glu-Ser-Glu-Ser-Glu-Ser.

SEQ ID NO: 41 is an amino acid sequence of an exemplary hydrophilic motif consisting of the amino acid sequence: Glu-Ser-Glu-Ser-Glu-Ser-Glu-Ser-Glu-Ser-Glu-Ser.

SEQ ID NO: 42 is an amino acid sequence of an exemplary hydrophilic motif consisting of the amino acid sequence: Glu-Ser-Glu-Ser-Glu-Ser-Glu-Ser-Glu-Ser-Glu-Ser-Glu-Ser.

SEQ ID NO: 43 is an amino acid sequence of an exemplary hydrophilic motif consisting of the amino acid sequence: Glu-Ser-Glu-Ser-Glu-Ser-Glu-Ser-Glu-Ser-Glu-Ser-Glu-Ser-Glu-Ser.

SEQ ID NO: 44 is an amino acid sequence of an exemplary hydrophilic motif consisting of the amino acid sequence: Glu-Glu.

SEQ ID NO: 45 is an amino acid sequence of an exemplary hydrophilic motif consisting of the amino acid sequence: Glu-Glu-Glu.

SEQ ID NO: 46 is an amino acid sequence of an exemplary hydrophilic motif consisting of the amino acid sequence: Glu-Glu-Glu-Glu.

SEQ ID NO: 47 is an amino acid sequence of an exemplary hydrophilic motif consisting of the amino acid sequence: Glu-Glu-Glu-Glu-Glu.

SEQ ID NO: 48 is an amino acid sequence of an exemplary hydrophilic motif consisting of the amino acid sequence: Glu-Glu-Glu-Glu-Glu-Glu.

SEQ ID NO: 49 is an amino acid sequence of an exemplary hydrophilic motif consisting of the amino acid sequence: Glu-Glu-Glu-Glu-Glu-Glu-Glu.

SEQ ID NO: 50 is an amino acid sequence of an exemplary hydrophilic motif consisting of the amino acid sequence: Glu-Glu-Glu-Glu-Glu-Glu-Glu-Glu.

SEQ ID NO: 51 is an amino acid sequence of an exemplary hydrophilic motif consisting of the amino acid sequence: Glu-Glu-Glu-Glu-Glu-Glu-Glu-Glu-Glu.

SEQ ID NO: 52 is an amino acid sequence of an exemplary hydrophilic motif consisting of the amino acid sequence: Glu-Glu-Glu-Glu-Glu-Glu-Glu-Glu-Glu-Glu.

SEQ ID NO: 53 is an amino acid sequence of an exemplary hydrophilic motif consisting of the amino acid sequence: Asp-Asp.

SEQ ID NO: 54 is an amino acid sequence of an exemplary hydrophilic motif consisting of the amino acid sequence: Asp-Asp-Asp.

SEQ ID NO: 55 is an amino acid sequence of an exemplary hydrophilic motif consisting of the amino acid sequence: Asp-Asp-Asp-Asp.

SEQ ID NO: 56 is an amino acid sequence of an exemplary hydrophilic motif consisting of the amino acid sequence: Asp-Asp-Asp-Asp-Asp.

SEQ ID NO: 57 is an amino acid sequence of an exemplary hydrophilic motif consisting of the amino acid sequence: Asp-Asp-Asp-Asp-Asp-Asp.

SEQ ID NO: 58 is an amino acid sequence of an exemplary hydrophilic motif consisting of the amino acid sequence: Asp-Asp-Asp-Asp-Asp-Asp-Asp.

SEQ ID NO: 59 is an amino acid sequence of an exemplary hydrophilic motif consisting of the amino acid sequence: Asp-Asp-Asp-Asp-Asp-Asp-Asp-Asp.

SEQ ID NO: 60 is an amino acid sequence of an exemplary hydrophilic motif consisting of the amino acid sequence: Asp-Asp-Asp-Asp-Asp-Asp-Asp-Asp-Asp.

SEQ ID NO: 61 is an amino acid sequence of an exemplary hydrophilic motif consisting of the amino acid sequence: Asp-Asp-Asp-Asp-Asp-Asp-Asp-Asp-Asp-Asp.

SEQ ID NO: 62 is an amino acid sequence of an exemplary hydrophilic motif consisting of the amino acid sequence: Glu-Asp.

SEQ ID NO: 63 is an amino acid sequence of an exemplary hydrophilic motif consisting of the amino acid sequence: Glu-Asp-Glu-Asp.

SEQ ID NO: 64 is an amino acid sequence of an exemplary hydrophilic motif consisting of the amino acid sequence: Glu-Asp-Glu-Asp-Glu-Asp.

SEQ ID NO: 65 is an amino acid sequence of an exemplary hydrophilic motif consisting of the amino acid sequence: Glu-Asp-Glu-Asp-Glu-Asp-Glu-Asp.

SEQ ID NO: 66 is an amino acid sequence of an exemplary hydrophilic motif consisting of the amino acid sequence: Glu-Asp-Glu-Asp-Glu-Asp-Glu-Asp-Glu-Asp.

SEQ ID NO: 67 is an amino acid sequence of an exemplary hydrophilic motif consisting of the amino acid sequence: Asp-Glu.

SEQ ID NO: 68 is an amino acid sequence of an exemplary hydrophilic motif consisting of the amino acid sequence: Asp-Glu-Asp-Glu.

SEQ ID NO: 69 is an amino acid sequence of an exemplary hydrophilic motif consisting of the amino acid sequence: Asp-Glu-Asp-Glu-Asp-Glu.

SEQ ID NO: 70 is an amino acid sequence of an exemplary hydrophilic motif consisting of the amino acid sequence: Asp-Glu-Asp-Glu-Asp-Glu-Asp-Glu.

SEQ ID NO: 71 is an amino acid sequence of an exemplary hydrophilic motif consisting of the amino acid sequence: Asp-Glu-Asp-Glu-Asp-Glu-Asp-Glu-Asp-Glu.

SEQ ID NO: 72 is an amino acid sequence of an exemplary hydrophilic motif consisting of the amino acid sequence: pSer-pSer-Gly-Ser-Gly-pSer-pSer.

SEQ ID NO: 73 is an amino acid sequence of an exemplary hydrophilic motif consisting of the amino acid sequence: Lys-Glu-Lys-Glu-Lys.

SEQ ID NO: 74 is an amino acid sequence of an exemplary hydrophilic motif consisting of the amino acid sequence: Lys-Glu-Lys-Glu-Lys-Glu-Lys.

SEQ ID NO: 75 is an amino acid sequence of an exemplary hydrophilic motif consisting of the amino acid sequence: Lys-Glu-Lys-Glu-Lys-Glu-Lys-Glu-Lys.

SEQ ID NO: 76 is an amino acid sequence of an exemplary hydrophilic motif consisting of the amino acid sequence: Lys-Glu-Lys-Glu-Lys-Glu-Lys-Glu-Lys-Glu-Lys.

SEQ ID NO: 77 is an amino acid sequence of an exemplary hydrophilic motif consisting of the amino acid sequence: Arg-Asp-Arg-Asp-Arg.

SEQ ID NO: 78 is an amino acid sequence of an exemplary hydrophilic motif consisting of the amino acid sequence: Arg-Asp-Arg-Asp-Arg-Asp-Arg.

SEQ ID NO: 79 is an amino acid sequence of an exemplary hydrophilic motif consisting of the amino acid sequence: Arg-Asp-Arg-Asp-Arg-Asp-Arg-Asp-Arg.

SEQ ID NO: 80 is an amino acid sequence of an exemplary hydrophilic motif consisting of the amino acid sequence: Arg-Asp-Arg-Asp-Asp-Arg-Asp-Arg-Asp-Arg-Arg.

SEQ ID NO: 81 is an amino acid sequence of an exemplary hydrophilic motif consisting of the amino acid sequence: Arg-Glu-Arg-Glu-Arg.

SEQ ID NO: 82 is an amino acid sequence of an exemplary hydrophilic motif consisting of the amino acid sequence: Arg-Glu-Arg-Glu-Arg-Glu-Arg.

SEQ ID NO: 83 is an amino acid sequence of an exemplary hydrophilic motif consisting of the amino acid sequence: Arg-Glu-Arg-Glu-Arg-Glu-Arg-Glu-Arg.

SEQ ID NO: 84 is an amino acid sequence of an exemplary hydrophilic motif consisting of the amino acid sequence: Arg-Glu-Arg-Glu-Arg-Glu-Arg-Glu-Arg-Glu-Arg.

SEQ ID NO: 85 is an amino acid sequence of an exemplary hydrophilic motif consisting of the amino acid sequence: Lys-Asp-Lys-Asp-Lys.

SEQ ID NO: 86 is an amino acid sequence of an exemplary hydrophilic motif consisting of the amino acid sequence: Lys-Asp-Lys-Asp-Lys-Asp-Lys.

SEQ ID NO: 87 is an amino acid sequence of an exemplary hydrophilic motif consisting of the amino acid sequence: Lys-Asp-Lys-Asp-Lys-Asp-Lys.

SEQ ID NO: 88 is an amino acid sequence of an exemplary hydrophilic motif consisting of the amino acid sequence: Lys-Asp-Lys-Asp-Lys-Asp-Lys.

SEQ ID NO: 89 is an amino acid sequence of an exemplary hydrophilic motif consisting of the amino acid sequence: pSer-Lys-pSer-Lys-Lys.

SEQ ID NO: 90 is an amino acid sequence of an exemplary hydrophilic motif consisting of the amino acid sequence: pSer-Lys-pSer-Lys-pSer-Lys-Lys.

SEQ ID NO: 91 is an amino acid sequence of an exemplary hydrophilic motif consisting of the amino acid sequence: pSer-Lys-pSer-Lys-pSer-Lys-pSer-Lys-Lys.

SEQ ID NO: 92 is an amino acid sequence of an exemplary hydrophilic motif consisting of the amino acid sequence: pSer-Lys-pSer-Lys-pSer-Lys-pSer-Lys-pSer-Lys-Lys.

SEQ ID NO: 93 is an amino acid sequence of an exemplary hydrophilic motif consisting of the amino acid sequence: pSer-Arg-pSer-Arg-Arg.

SEQ ID NO: 94 is an amino acid sequence of an exemplary hydrophilic motif consisting of the amino acid sequence: pSer-Arg-pSer-Arg-pSer-Arg-Arg.

SEQ ID NO: 95 is an amino acid sequence of an exemplary hydrophilic motif consisting of the amino acid sequence: pSer-Arg-pSer-Arg-pSer-Arg-pSer-Arg-Arg.

SEQ ID NO: 96 is an amino acid sequence of an exemplary hydrophilic motif consisting of the amino acid sequence: pSer-Arg-pSer-Arg-pSer-Arg-pSer-Arg-pSer-Arg-Arg.

SEQ ID NO: 97 is an amino acid sequence of an exemplary hydrophilic motif consisting of the amino acid sequence: Ser-Lys-Asp-Ser-Lys-Asp-Lys.

SEQ ID NO: 98 is an amino acid sequence of an exemplary hydrophilic motif consisting of the amino acid sequence: Ser-Lys-Asp-Ser-Lys-Asp-Ser-Lys-Asp-Lys.

SEQ ID NO: 99 is an amino acid sequence of an exemplary hydrophilic motif consisting of the amino acid sequence: Ser-Lys-Asp-Ser-Lys-Asp-Ser-Lys-Asp-Ser-Lys-Asp-Lys.

SEQ ID NO: 100 is an amino acid sequence of an exemplary hydrophilic motif consisting of the amino acid sequence: Ser-Arg-Glu-Ser-Arg-Glu-Arg.

SEQ ID NO: 101 is an amino acid sequence of an exemplary hydrophilic motif consisting of the amino acid sequence: Ser-Arg-Glu-Ser-Arg-Glu-Ser-Arg-Glu-Arg.

SEQ ID NO: 102 is an amino acid sequence of an exemplary hydrophilic motif consisting of the amino acid sequence: Ser-Arg-Glu-Ser-Arg-Glu-Ser-Arg-Glu-Ser-Arg-Glu-Arg.

SEQ ID NO: 103 is an amino acid sequence of an exemplary hydrophilic motif consisting of the amino acid sequence: Ser-Lys-Glu-Ser-Lys-Glu-Lys.

SEQ ID NO: 104 is an amino acid sequence of an exemplary hydrophilic motif consisting of the amino acid sequence: Ser-Lys-Glu-Ser-Lys-Glu-Ser-Lys-Glu-Lys.

SEQ ID NO: 105 is an amino acid sequence of an exemplary hydrophilic motif consisting of the amino acid sequence: Ser-Lys-Glu-Ser-Lys-Glu-Ser-Lys-Glu-Ser-Lys-Glu-Lys.

SEQ ID NO: 106 is an amino acid sequence of an exemplary hydrophilic motif consisting of the amino acid sequence: Ser-Arg-Asp-Ser-Arg-Asp-Arg.

SEQ ID NO: 107 is an amino acid sequence of an exemplary hydrophilic motif consisting of the amino acid sequence: Ser-Arg-Asp-Ser-Arg-Asp-Ser-Arg-Asp-Arg.

SEQ ID NO: 108 is an amino acid sequence of an exemplary hydrophilic motif consisting of the amino acid sequence: Ser-Arg-Asp-Ser-Arg-Asp-Ser-Arg-Asp-Ser-Arg-Asp-Arg.

SEQ ID NO: 109 is an amino acid sequence of an exemplary hydrophilic motif consisting of the amino acid sequence: Lys-Glu-Lys-Glu, in which the C-terminus of the amino acid sequence is amidated.

SEQ ID NO: 110 is an amino acid sequence of an exemplary hydrophilic motif consisting of the amino acid sequence: Lys-Glu-Lys-Glu-Lys-Glu, in which the C-terminus of the amino acid sequence is amidated.

SEQ ID NO: 111 is an amino acid sequence of an exemplary hydrophilic motif consisting of the amino acid sequence: Lys-Glu-Lys-Glu-Lys-Glu-Lys-Glu, in which the C-terminus of the amino acid sequence is amidated.

SEQ ID NO: 112 is an amino acid sequence of an exemplary hydrophilic motif consisting of the amino acid sequence: Lys-Glu-Lys-Glu-Lys-Glu-Lys-Glu-Lys-Glu, in which the C-terminus of the amino acid sequence is amidated.

SEQ ID NO: 113 is an amino acid sequence of an exemplary hydrophilic motif consisting of the amino acid sequence: Arg-Asp-Arg-Asp, in which the C-terminus of the amino acid sequence is amidated.

SEQ ID NO: 114 is an amino acid sequence of an exemplary hydrophilic motif consisting of the amino acid sequence: Arg-Asp-Arg-Asp-Arg-Asp, in which the C-terminus of the amino acid sequence is amidated.

SEQ ID NO: 115 is an amino acid sequence of an exemplary hydrophilic motif consisting of the amino acid sequence: Arg-Asp-Arg-Asp-Arg-Asp-Arg-Asp, in which the C-terminus of the amino acid sequence is amidated.

SEQ ID NO: 116 is an amino acid sequence of an exemplary hydrophilic motif consisting of the amino acid sequence: Arg-Asp-Arg-Asp-Asp-Arg-Asp-Arg-Asp-Arg, in which the C-terminus of the amino acid sequence is amidated.

SEQ ID NO: 117 is an amino acid sequence of an exemplary hydrophilic motif consisting of the amino acid sequence: Arg-Glu-Arg-Glu, in which the C-terminus of the amino acid sequence is amidated.

SEQ ID NO: 118 is an amino acid sequence of an exemplary hydrophilic motif consisting of the amino acid sequence: Arg-Glu-Arg-Glu-Arg-Glu, in which the C-terminus of the amino acid sequence is amidated.

SEQ ID NO: 119 is an amino acid sequence of an exemplary hydrophilic motif consisting of the amino acid sequence: Arg-Glu-Arg-Glu-Arg-Glu-Arg-Glu, in which the C-terminus of the amino acid sequence is amidated.

SEQ ID NO: 120 is an amino acid sequence of an exemplary hydrophilic motif consisting of the amino acid sequence: Arg-Glu-Arg-Glu-Arg-Glu-Arg-Glu-Arg-Glu, in which the C-terminus of the amino acid sequence is amidated.

SEQ ID NO: 121 is an amino acid sequence of an exemplary hydrophilic motif consisting of the amino acid sequence: Lys-Asp-Lys-Asp, in which the C-terminus of the amino acid sequence is amidated.

SEQ ID NO: 122 is an amino acid sequence of an exemplary hydrophilic motif consisting of the amino acid sequence: Lys-Asp-Lys-Asp-Lys-Asp, in which the C-terminus of the amino acid sequence is amidated.

SEQ ID NO: 123 is an amino acid sequence of an exemplary hydrophilic motif consisting of the amino acid sequence: Lys-Asp-Lys-Asp-Lys-Asp, in which the C-terminus of the amino acid sequence is amidated.

SEQ ID NO: 124 is an amino acid sequence of an exemplary hydrophilic motif consisting of the amino acid sequence: Lys-Asp-Lys-Asp-Lys-Asp, in which the C-terminus of the amino acid sequence is amidated.

SEQ ID NO: 125 is an amino acid sequence of an exemplary hydrophilic motif consisting of the amino acid sequence: pSer-Lys-pSer-Lys, in which the C-terminus of the amino acid sequence is amidated.

SEQ ID NO: 126 is an amino acid sequence of an exemplary hydrophilic motif consisting of the amino acid sequence: pSer-Lys-pSer-Lys-pSer-Lys, in which the C-terminus of the amino acid sequence is amidated.

SEQ ID NO: 127 is an amino acid sequence of an exemplary hydrophilic motif consisting of the amino acid sequence: pSer-Lys-pSer-Lys-pSer-Lys-pSer-Lys, in which the C-terminus of the amino acid sequence is amidated.

SEQ ID NO: 128 is an amino acid sequence of an exemplary hydrophilic motif consisting of the amino acid sequence: pSer-Lys-pSer-Lys-pSer-Lys-pSer-Lys-pSer-Lys, in which the C-terminus of the amino acid sequence is amidated.

SEQ ID NO: 129 is an amino acid sequence of an exemplary hydrophilic motif consisting of the amino acid sequence: pSer-Arg-pSer-Arg, in which the C-terminus of the amino acid sequence is amidated.

SEQ ID NO: 130 is an amino acid sequence of an exemplary hydrophilic motif consisting of the amino acid sequence: pSer-Arg-pSer-Arg, in which the C-terminus of the amino acid sequence is amidated.

SEQ ID NO: 131 is an amino acid sequence of an exemplary hydrophilic motif consisting of the amino acid sequence: pSer-Arg-pSer-Arg-pSer-Arg-pSer-Arg, in which the C-terminus of the amino acid sequence is amidated.

SEQ ID NO: 132 is an amino acid sequence of an exemplary hydrophilic motif consisting of the amino acid sequence: pSer-Arg-pSer-Arg-pSer-Arg-pSer-Arg-pSer-Arg, in which the C-terminus of the amino acid sequence is amidated.

SEQ ID NO: 133 is an amino acid sequence of an exemplary hydrophilic motif consisting of the amino acid sequence: Ser-Lys-Asp-Ser-Lys-Asp, in which the C-terminus of the amino acid sequence is amidated.

SEQ ID NO: 134 is an amino acid sequence of an exemplary hydrophilic motif consisting of the amino acid sequence: Ser-Lys-Asp-Ser-Lys-Asp-Ser-Lys-Asp, in which the C-terminus of the amino acid sequence is amidated.

SEQ ID NO: 135 is an amino acid sequence of an exemplary hydrophilic motif consisting of the amino acid sequence: Ser-Lys-Asp-Ser-Lys-Asp-Ser-Lys-Asp-Ser-Lys-Asp, in which the C-terminus of the amino acid sequence is amidated.

SEQ ID NO: 136 is an amino acid sequence of an exemplary hydrophilic motif consisting of the amino acid sequence: Ser-Arg-Glu-Ser-Arg-Glu, in which the C-terminus of the amino acid sequence is amidated.

SEQ ID NO: 137 is an amino acid sequence of an exemplary hydrophilic motif consisting of the amino acid sequence: Ser-Arg-Glu-Ser-Arg-Glu-Ser-Arg-Glu, in which the C-terminus of the amino acid sequence is amidated.

SEQ ID NO: 138 is an amino acid sequence of an exemplary hydrophilic motif consisting of the amino acid sequence: Ser-Arg-Glu-Ser-Arg-Glu-Ser-Arg-Glu-Ser-Arg-Glu, in which the C-terminus of the amino acid sequence is amidated.

SEQ ID NO: 139 is an amino acid sequence of an exemplary hydrophilic motif consisting of the amino acid sequence: Ser-Lys-Glu-Ser-Lys-Glu, in which the C-terminus of the amino acid sequence is amidated.

SEQ ID NO: 140 is an amino acid sequence of an exemplary hydrophilic motif consisting of the amino acid sequence: Ser-Lys-Glu-Ser-Lys-Glu-Ser-Lys-Glu, in which the C-terminus of the amino acid sequence is amidated.

SEQ ID NO: 141 is an amino acid sequence of an exemplary hydrophilic motif consisting of the amino acid sequence: Ser-Lys-Glu-Ser-Lys-Glu-Ser-Lys-Glu-Ser-Lys-Glu, in which the C-terminus of the amino acid sequence is amidated.

SEQ ID NO: 142 is an amino acid sequence of an exemplary hydrophilic motif consisting of the amino acid sequence: Ser-Arg-Asp-Ser-Arg-Asp, in which the C-terminus of the amino acid sequence is amidated.

SEQ ID NO: 143 is an amino acid sequence of an exemplary hydrophilic motif consisting of the amino acid sequence: Ser-Arg-Asp-Ser-Arg-Asp-Ser-Arg-Asp, in which the C-terminus of the amino acid sequence is amidated.

SEQ ID NO: 144 is an amino acid sequence of an exemplary hydrophilic motif consisting of the amino acid sequence: Ser-Arg-Asp-Ser-Arg-Asp-Ser-Arg-Asp-Ser-Arg-Asp, in which the C-terminus of the amino acid sequence is amidated.

SEQ ID NO: 145 is an amino acid sequence of an exemplary protease substrate motif consisting of the amino acid sequence: Ala-Ala-Asn-Gly, which comprises a protease recognition site of legumain.

SEQ ID NO: 146 is an amino acid sequence of an exemplary protease substrate motif consisting of the amino acid sequence: Leu-Ala-Gly-Gly-Ala-Gly, which comprises a protease recognition site of cathepsin B.

SEQ ID NO: 147 is an amino acid sequence of an exemplary protease substrate motif consisting of the amino acid sequence: Arg-Ser-Lys-Arg-Val-Ser-Gly, which comprises a protease recognition site of a furin protease.

SEQ ID NO: 148 is an amino acid sequence of an exemplary protease substrate motif consisting of the amino acid sequence: Arg-Ser-Lys-Arg-Ser, which comprises a protease recognition site of a furin protease.

SEQ ID NO: 149 is an amino acid sequence of an exemplary protease substrate motif consisting of the amino acid sequence: Ser-Ala-Gln-Ala-Val-Val-Ser-Gln, which comprises a protease recognition site of an ADAM10 protease.

SEQ ID NO: 150 is an amino acid sequence of an exemplary protease substrate motif consisting of the amino acid sequence: Ala-Gin-Ala-Val-Val-Ser, which comprises a protease recognition site of an ADAM10 protease.

SEQ ID NO: 151 is an amino acid sequence of an exemplary protease substrate motif consisting of the amino acid sequence: Leu-Ala-Gln-Ala-Val-Val-Ser-Ala, which comprises a protease recognition site of a TACE protease.

SEQ ID NO: 152 is an amino acid sequence of an exemplary protease substrate motif consisting of the amino acid sequence: Ala-Gin-Ala-Val-Val-Ser, which comprises a protease recognition site of a TACE protease.

SEQ ID NO: 153 is an amino acid sequence of an exemplary protease substrate motif consisting of the amino acid sequence: Leu-Ala-Ala-Ala-Val-Val-Ser-Ser, which comprises a protease recognition site of a TACE protease.

SEQ ID NO: 154 is an amino acid sequence of an exemplary protease substrate motif consisting of the amino acid sequence: Ala-Ala-Ala-Val-Val, which comprises a protease recognition site of a TACE protease.

SEQ ID NO: 155 is an amino acid sequence of an exemplary protease substrate motif consisting of the amino acid sequence: Pro-Ala-Ala-Ala-Gln-Arg-Leu-Arg, which comprises a protease recognition site of an ADAM8 protease.

SEQ ID NO: 156 is an amino acid sequence of an exemplary protease substrate motif consisting of the amino acid sequence: Ala-Ala-Ala-Gln-Arg-Leu, which comprises a protease recognition site of an ADAM8 protease.

SEQ ID NO: 157 is an amino acid sequence of an exemplary protease substrate motif consisting of the amino acid sequence: Leu-Pro-Ala-Ala-Leu-Val-Gly-Ala, which comprises a protease recognition site of a MMP-2 protease.

SEQ ID NO: 158 is an amino acid sequence of an exemplary protease substrate motif consisting of the amino acid sequence: Pro-Ala-Ala-Leu, which comprises a protease recognition site of a MMP-2 protease.

SEQ ID NO: 159 is an amino acid sequence of an exemplary protease substrate motif consisting of the amino acid sequence: Leu-Pro-Ser-Gly-Leu-Val-Gly-Ala, which comprises a protease recognition site of a MMP-2 protease.

SEQ ID NO: 160 is an amino acid sequence of an exemplary protease substrate motif consisting of the amino acid sequence: Pro-Ser-Gly-Leu, which comprises a protease recognition site of a MMP-2 protease.

SEQ ID NO: 161 is an amino acid sequence of an exemplary protease substrate motif consisting of the amino acid sequence: Gly-Pro-Ala-Gly-Leu-Ala-Gly-Ala, which comprises a protease recognition site of a MMP-9 protease.

SEQ ID NO: 162 is an amino acid sequence of an exemplary protease substrate motif consisting of the amino acid sequence: Pro-Ala-Gly-Leu, which comprises a protease recognition site of a MMP-9 protease.

SEQ ID NO: 163 is an amino acid sequence of an exemplary protease substrate motif consisting of the amino acid sequence: Gly-Pro-Gly-Gly-Leu-Ala-Gly-Ala, which comprises a protease recognition site of a MMP-9 protease.

SEQ ID NO: 164 is an amino acid sequence of an exemplary protease substrate motif consisting of the amino acid sequence: Gly-Pro-Leu-Gly-Leu-Val-Gly-Gln, which comprises a protease recognition site of a MMP-1 protease.

SEQ ID NO: 165 is an amino acid sequence of an exemplary protease substrate motif consisting of the amino acid sequence: Pro-Leu-Gly-Leu, which comprises a protease recognition site of a MMP-1 protease.

SEQ ID NO: 166 is an amino acid sequence of an exemplary protease substrate motif consisting of the amino acid sequence: Gly-Pro-Ala-Gly-Leu-Gly-Gly-Gly, which comprises a protease recognition site of a MMP-7 protease.

SEQ ID NO: 167 is an amino acid sequence of an exemplary protease substrate motif consisting of the amino acid sequence: Pro-Ala-Gly-Leu, which comprises a protease recognition site of a MMP-7 protease.

SEQ ID NO: 168 is an amino acid sequence of an exemplary protease substrate motif consisting of the amino acid sequence: Gly-Pro-Pro-Gly-Leu-Arg-Gly-Pro, which comprises a protease recognition site of a MMP-13 protease.

SEQ ID NO: 169 is an amino acid sequence of an exemplary protease substrate motif consisting of the amino acid sequence: Pro-Pro-Gly-Leu, which comprises a protease recognition site of a MMP-13 protease.

SEQ ID NO: 170 is an amino acid sequence of an exemplary protease substrate motif consisting of the amino acid sequence: Gly-Pro-Leu-Gly-Leu-Arg-Gly-Pro, which comprises a protease recognition site of a MMP-13 protease.

SEQ ID NO: 171 is an amino acid sequence of an exemplary protease substrate motif consisting of the amino acid sequence: Pro-Leu-Gly-Leu, which comprises a protease recognition site of a MMP-13 protease.

SEQ ID NO: 172 is an amino acid sequence of an exemplary protease substrate motif consisting of the amino acid sequence: Gly-Pro-Ala-Gly-Leu-Arg-Thr-Glu, which comprises a protease recognition site of a MMP-14 protease.

SEQ ID NO: 173 is an amino acid sequence of an exemplary protease substrate motif consisting of the amino acid sequence: Leu-Pro-Gln-Gly-Leu-Ala-Gly-Arg, which comprises a protease recognition site of a MMP-14 protease.

SEQ ID NO: 174 is an amino acid sequence of an exemplary protease substrate motif consisting of the amino acid sequence: Pro-Ala-Gly-Leu, which comprises a protease recognition site of a MMP-14 protease.

SEQ ID NO: 175 is an amino acid sequence of an exemplary protease substrate motif consisting of the amino acid sequence: Glu-Ala-Glu-Asn-Gly-Glu-Leu-Pro, which comprises a protease recognition site of a LGMN protease.

SEQ ID NO: 176 is an amino acid sequence of an exemplary protease substrate motif consisting of the amino acid sequence: Ala-Ala-Asn-Gly, which comprises a protease recognition site of a LGMN protease.

SEQ ID NO: 177 is an amino acid sequence of an exemplary protease substrate motif consisting of the amino acid sequence: Asp-Asn-Phe-Leu-Val, which comprises a protease recognition site of a Cathepsin A protease.

SEQ ID NO: 178 is an amino acid sequence of an exemplary protease substrate motif consisting of the amino acid sequence: Asp-Asn-Phe-Phe-Val, which comprises a protease recognition site of a Cathepsin A protease.

SEQ ID NO: 179 is an amino acid sequence of an exemplary protease substrate motif consisting of the amino acid sequence: Gly-Leu-Ala-Gly-Gly-Ala-Gly-Gly, which comprises a protease recognition site of a Cathepsin B protease.

SEQ ID NO: 180 is an amino acid sequence of an exemplary protease substrate motif consisting of the amino acid sequence: Leu-Ala-Gly-Gly-Ala-Gly, which comprises a protease recognition site of a Cathepsin B protease.

SEQ ID NO: 181 is an amino acid sequence of an exemplary protease substrate motif consisting of the amino acid sequence: Gly-Leu-Val-Ala-Leu-Leu-Ala-Gly-Gly, which comprises a protease recognition site of a Cathepsin B protease.

SEQ ID NO: 182 is an amino acid sequence of an exemplary protease substrate motif consisting of the amino acid sequence: Leu-Glu-Val-Leu-Ile-Val, which comprises a protease recognition site of a Cathepsin D protease.

SEQ ID NO: 183 is an amino acid sequence of an exemplary protease substrate motif consisting of the amino acid sequence: Glu-Val-Leu-Ile-Val, which comprises a protease recognition site of a Cathepsin D protease.

SEQ ID NO: 184 is an amino acid sequence of an exemplary protease substrate motif consisting of the amino acid sequence: Glu-Val-Val-Leu-Val-Ala-Leu-Ala, which comprises a protease recognition site of a Cathepsin E protease.

SEQ ID NO: 185 is an amino acid sequence of an exemplary protease substrate motif consisting of the amino acid sequence: Glu-Val-Val-Phe-Val-Ala-Leu-Ala, which comprises a protease recognition site of a Cathepsin E protease.

SEQ ID NO: 186 is an amino acid sequence of an exemplary protease substrate motif consisting of the amino acid sequence: Val-Leu-Val-Ala, which comprises a protease recognition site of a Cathepsin E protease.

SEQ ID NO: 187 is an amino acid sequence of an exemplary protease substrate motif consisting of the amino acid sequence: Val-Phe-Val-Ala, which comprises a protease recognition site of a Cathepsin E protease.

SEQ ID NO: 188 is an amino acid sequence of an exemplary protease substrate motif consisting of the amino acid sequence: Asp-Val-Leu-Leu-Ser-Trp-Ala-Val, which comprises a protease recognition site of a Cathepsin G protease.

SEQ ID NO: 189 is an amino acid sequence of an exemplary protease substrate motif consisting of the amino acid sequence: Val-Leu-Leu-Ser-Trp, which comprises a protease recognition site of a Cathepsin G protease.

SEQ ID NO: 190 is an amino acid sequence of an exemplary protease substrate motif consisting of the amino acid sequence: Ala-Lys-Leu-Lys-Glu-Glu-Asp-Asp, which comprises a protease recognition site of a Cathepsin K protease.

SEQ ID NO: 191 is an amino acid sequence of an exemplary protease substrate motif consisting of the amino acid sequence: Ala-Gly-Leu-Gly-Glu-Glu-Asp-Asp, which comprises a protease recognition site of a Cathepsin K protease.

SEQ ID NO: 192 is an amino acid sequence of an exemplary protease substrate motif consisting of the amino acid sequence: Ala-Leu-Leu-Gly-Ala-Pro-Pro-Pro, which comprises a protease recognition site of a Cathepsin L protease.

SEQ ID NO: 193 is an amino acid sequence of an exemplary protease substrate motif consisting of the amino acid sequence: Gly-Leu-Leu-Gly-Ser-Glu-Pro-Glu, which comprises a protease recognition site of a Cathepsin L protease.

SEQ ID NO: 194 is an amino acid sequence of an exemplary protease substrate motif consisting of the amino acid sequence: Leu-Gly-Ala-Pro, which comprises a protease recognition site of a Cathepsin L protease.

SEQ ID NO: 195 is an amino acid sequence of an exemplary protease substrate motif consisting of the amino acid sequence: Leu-Gly-Ser-Glu, which comprises a protease recognition site of a Cathepsin L protease.

SEQ ID NO: 196 is an amino acid sequence of an exemplary protease substrate motif consisting of the amino acid sequence: Ala-Ala-Lys-Gly-Ala-Ala-Pro-Glu, which comprises a protease recognition site of a Cathepsin S protease.

SEQ ID NO: 197 is an amino acid sequence of an exemplary protease substrate motif consisting of the amino acid sequence: Leu-Gly-Ala-Ala, which comprises a protease recognition site of a Cathepsin S protease.

SEQ ID NO: 198 is an amino acid sequence of an exemplary protease substrate motif consisting of the amino acid sequence: Ser-Ser-Gln-Tyr-Ser-Ser-Asn-Gly, which comprises a protease recognition site of a KLK3 protease.

SEQ ID NO: 199 is an amino acid sequence of an exemplary protease substrate motif consisting of the amino acid sequence: Ser-Gln-Gln-Tyr-Ser-Ser-Asn-Gly, which comprises a protease recognition site of a KLK3 protease.

SEQ ID NO: 200 is an amino acid sequence of an exemplary protease substrate motif consisting of the amino acid sequence: Ser-Ser-Gln-Gln-Ser-Ser-Asn-Gly, which comprises a protease recognition site of a KLK3 protease.

SEQ ID NO: 201 is an amino acid sequence of an exemplary protease substrate motif consisting of the amino acid sequence: Gly-Gly-Ser-Arg-Ser-Gly-Gly-Gly, which comprises a protease recognition site of a KLK2 protease.

SEQ ID NO: 202 is an amino acid sequence of an exemplary protease substrate motif consisting of the amino acid sequence: Gly-Gly-Ser-Arg-Ser-Pro-Gly-Gly, which comprises a protease recognition site of a KLK2 protease.

SEQ ID NO: 203 is an amino acid sequence of an exemplary protease substrate motif consisting of the amino acid sequence: Gly-Val-Asn-Leu-Asp-Val-Glu-Val, which comprises a protease recognition site of a beta-secretase 1 protease.

SEQ ID NO: 204 is an amino acid sequence of an exemplary protease substrate motif consisting of the amino acid sequence: Arg-Gln-Ala-Arg-Lys-Val-Gly-Gly, which comprises a protease recognition site of a matriptase-1 protease.

SEQ ID NO: 205 is an amino acid sequence of an exemplary protease substrate motif consisting of the amino acid sequence: Ala-Ala-Ala-Arg-Lys-Val-Gly-Gly, which comprises a protease recognition site of a matriptase-1 protease.

SEQ ID NO: 206 is an amino acid sequence of protein 1 consisting of the amino acid sequence: Phe-Lys-Phe-Glu-Ala-Ala-Asn-Gly-Glu-Glu-Gly-Ser-Gly-Glu-Glu, in which the N-terminus is modified to comprise a Fmoc protecting group.

SEQ ID NO: 207 is an amino acid sequence of protein 2 consisting of the amino acid sequence: Phe-Lys-Phe-Glu-Ala-Ala-Asn, in which the N-terminus is modified to comprise a Fmoc protecting group.

SEQ ID NO: 208 is an amino acid sequence of protein 3 consisting of the amino acid sequence: Phe-Lys-Phe-Glu-Leu-Ala-Gly-Gly-Ala-Gly-Glu-Glu-Gly-Ser-Gly-Glu-Glu-Glu, in which the N-terminus is modified to comprise a Fmoc protecting group.

### DETAILED DESCRIPTION

As used herein, “4-{4-[1-(9-Fluorenylmethyloxycarbonylamino)ethyl]-2-methoxy-5-nitrophenoxy}butanoic acid” refers to a fluorenylmethoxycarbonyl protecting group (Fmoc) (CAS 162827-98-7).

As used herein, the singular forms “a,” “an,” and “the” include the plural referents unless the context clearly indicates otherwise. The terms “include” and “such as” are intended to convey inclusion without limitation, unless otherwise specifically indicated otherwise.

As used herein, “about” or “approximately” may be used interchangeably and refer to within an acceptable error range for the particular value as determined by skilled persons which will depend in part on how the value is measured or determined, e.g., the limitations of the measurement system. Where particular values are described in the application and claims, unless otherwise stated, the term “about” should be assumed to mean an acceptable error range for the particular value.

As used herein, “activation” refers to rendering molecules capable of reaction or to increase the reactivity of substrate molecules by the presence of other molecules, moieties, motifs, domains, or functional groups proximal to the substrate molecules.

As used herein, “amino acid” refers to naturally-occurring α-amino acids and their stereoisomers, as well as unnatural (non-naturally occurring) amino acids and their stereoisomers. “Stereoisomers” of amino acids refers to mirror image isomers of the amino acids, such as L-amino acids or D-amino acids. For example, a stereoisomer of a naturally-occurring amino acid refers to the mirror image isomer of the naturally-occurring amino acid, i.e., the D-amino acid. Naturally-occurring amino acids are those encoded by the genetic code, as well as those amino acids that are later modified, e.g., hydroxyproline, γ-carboxyglutamate and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (lie), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof.

Stereoisomers of naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof. Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” are unnatural amino acids that have the same basic chemical structure as naturally-occurring amino acids, i.e., an a carbon that is bound to a hydrogen, a carboxyl group, an amino group, but have modified R (i.e., side-chain) groups or modified peptide backbones, e.g., homoserine, norleucine, methionine sulfoxide, methionine methyl sulfonium. Amino acids may be referred to herein by either their commonly known three letter symbols or by the one-letter symbols recommended by the IUPAC-IUB Biochemical Nomenclature Commission. For example, an L-amino acid may be represented herein by its commonly known three letter symbol (e.g., Arg for L-arginine) or by an upper-case one-letter amino acid symbol (e.g., R for L-arginine). A D-amino acid may be represented herein by its commonly known three letter symbol (e.g., D-Arg for D-arginine) or by a lower-case one-letter amino acid symbol (e.g., r for D-arginine). Skilled persons will understand that an amino acid residue (typically serine, threonine, or tyrosine residues) may be modified by phosphorylation. As used herein, an amino acid residue designated “p(Xaa)” refers to a phosphorylated amino acid residue (e.g., pCys, pLys, pArg, etc. . . . ).

As used herein, “amino acid sequence” refers to the order of amino acids as they occur in a polypeptide. Unless otherwise stated, skilled persons will understand that the order of an amino acid sequence forming a polypeptide is written from the N-terminus to the C-terminus of the polypeptide. With respect to amino acid sequences, one of skill in the art will recognize that individual substitutions, additions, or deletions to a peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. The chemically similar amino acid includes, without limitation, a naturally-occurring amino acid such as an L-amino acid, a stereoisomer of a naturally occurring amino acid such as a D-amino acid, and an unnatural amino acid such as an amino acid analog, amino acid mimetic, synthetic amino acid, N-substituted glycine, and N-methyl amino acid.

Conservative substitution tables providing functionally similar amino acids are well known in the art. For example, substitutions may be made wherein an aliphatic amino acid (e.g., G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some embodiments, an amino acid with an acidic side chain, e.g., E or D, may be substituted with its uncharged counterpart, e.g., Q or N, respectively; or vice versa. Each of the following eight groups contains other exemplary amino acids that are conservative substitutions for one another: 1) Alanine (A)|Glycine (G); 2) Aspartic acid (D)|Glutamic acid (E); 3) Asparagine (N)|Glutamine (Q); 4) Arginine (R)|Lysine (K); 5) Isoleucine (I)|Leucine (L)|Methionine (M)|Valine (V); 6) Phenylalanine (F)|Tyrosine (Y)|Tryptophan (W); 7) Serine (S)|Threonine (T); and, 8) Cysteine (C)|Methionine (M) (see, e.g., Creighton, Proteins, 1993).

Chemical polypeptide synthesis in general is well-known in the art and usually proceeds from the polypeptide's C-terminus to the N-terminus (cf., brochure “Solid Phase Peptide Synthesis Bachem—Pioneering Partner for Peptides”, published by Global Marketing, Bachem group, June 2014). During synthesis, formation of the peptide bond between the alpha amino group of the first amino acid and the alpha carboxyl group of a second amino acid should be favored over unintended side reactions. This is commonly achieved by the use of “permanent” and “temporary” protecting groups. The former are used to block reactive amino acid side chains and the C-terminal carboxyl group of the growing peptide chain and are only removed at the end of the entire synthesis. The latter are used to block the alpha amino group of the second amino acid during the coupling step, thereby avoiding, e.g., peptide bond formation between multiple copies of the second amino acid. Two standard approaches to chemical peptide synthesis can be distinguished, namely Liquid Phase Peptide Synthesis (LPPS) and Solid Phase Peptide Synthesis (SPPS). LPPS, also referred to as Solution Peptide Synthesis, takes place in a homogenous reaction medium. Successive couplings yield the desired peptide. LPPS usually involves the isolation, characterization, and—where desired—purification of intermediates after each coupling. In SPPS, a peptide anchored by its C-terminus to an insoluble polymer resin is assembled by the successive addition of the protected amino acids constituting its sequence. Skilled persons will understand that custom polypeptide synthesis services are readily commercially available (e.g., Thermo Scientific Peptide Synthesis Quote Form (v20150818) Thermo Fisher Scientific. 168 Third Avenue. Waltham, MA USA 02451).

As used herein, “anti-parallel β-sheet structure” refers to a β-sheet motif comprising β-strands in an anti-parallel arrangement.

As used herein, “aqueous milieu” refers to the physical environment of an aqueous solution comprising one or more solutes. For example, skilled persons will understand that an aqueous milieu may include in vitro or in vivo physical environments, such an assay buffer or a plasma, respectively.

As used herein, “β-sheet” refers to a protein secondary structure motif comprising two or more β-strands in which each β-strand bonds intramolecularly to another β-strand by two or more hydrogen bonds.

As used herein, “β-strand motif” refers to a polypeptide motif comprising a pleated linear arrangement of amino acid residues in which the side-chains of the amino acid residues alternate above and below the backbone of the polypeptide (Cheng P. N. et al, The Supramolecular Chemistry of β-Sheets, J. Am. Chem. Soc., 135, 5477-5492 (2013); which is hereby incorporated by reference in its entirety). Skilled persons will understand that a β-strand typically comprises 3 to 10 amino acids residues and may form hydrogen bonds with adjacent β-strands in an anti-parallel arrangement, parallel arrangement, or a mix of anti-parallel and parallel arrangements. In the anti-parallel arrangement, successive β-strands alternate directions so that the N-terminus of one β-strand is adjacent to the C-terminus of the next β-strand. The anti-parallel arrangement generates an inter-strand stability by allowing the inter-strand hydrogen bonds between carbonyls and amines to be planar, with the peptide backbone dihedral angles (φ, ψ) being, respectively, about 140° and about 135°.

As used herein, “configured to self-assemble” refers to a polypeptide motif having an amino acid sequence configured such that, upon its dissociation, will form polypeptide secondary structure with other disorganized nominally identical polypeptide motifs to form an organized supramolecular structure spontaneously through non-covalent interactions (e.g., hydrogen bonding, hydrophobic interactions, and electrostatic attraction). For example, in some embodiments, a β-strand motif dissociated by protease cleavage will form a β-sheet structure with other disorganized nominally identical β-strand motifs.

As used herein, “crosslinker” refers to a molecule that comprises a reactive group or residue capable of chemically attaching to the specific functional groups of other molecules, such as proteins.

As used herein, “configured” refers to the selective arrangement, form, or order of a composition of matter.

As used herein, “construct” refers to a composition of matter formed, made, or created by combining parts or elements.

As used herein, “domain” refers to a distinct functional and/or structural unit of a polypeptide. For example, skilled persons will understand that a domain may include any portion of a polypeptide that is self-stabilizing and folds into its tertiary structure independently from the rest of the polypeptide.

As used herein, “hydrophilic motif” refers to a polypeptide motif configured to be soluble in water or any other composition of aqueous milieu. For example, a hydrophilic motif may have a net negative charge or comprise a zwitterion to facilitate solubility.

As used herein, “intermolecular interaction” refers to an interaction between two or more molecules not covalently bound to each other.

As used herein, “intramolecular interaction” refers to an interaction between two covalently bound molecules.

As used herein, “irreversible bond” refers to a chemical bond having a sufficiently high enough activation energy to not to react in a context.

As used herein, “ligand” refers to a molecule that binds to another molecule.

As used herein, “linker” refers to a molecule that covalently joins at least two other molecules.

As used herein, “moiety” refers to one of a part or portion of a molecule into which the molecule is divided. For example, skilled persons understand that a hemoglobin molecule comprises four heme moieties.

As used herein, “molecule” refers to one or more atoms bound to together, representing the smallest unit of a compound that can take part in a chemical reaction. As used herein, “motif” refers to a distinctive, sometimes recurrent, pattern in the sequence (i.e., primary structure) or spatial relationship (i.e., secondary structure) of a polymer. For example, as used herein, a “tri-glycine motif” refers to a portion of a polypeptide sequence consisting of three consecutive glycine molecules.

As used herein, “nominally identical β-strand motifs” refers to β-strand motifs having, from N-Terminus to C-Terminus, the same amino acid sequence.

As used herein, “non-covalent bond” refers to a chemical bond involving any combination of electrostatic, hydrogen bond, van der Waals, hydrophobic, hydrophilic, or induced dipole interactions between atoms.

As used herein, “operatively connected” refers to the joining or binding of two molecules either via a linker or directly to each other.

As used herein, “polymer” refers to any of a class of natural or synthetic substances composed of two or more chemical units (e.g., “monomers”). Polymers include, for example, proteins and nucleic acids.

As used herein, “protease cleavage site” refers to the location on a substrate in which a protease cleaves the substrate. Skilled persons will understand that the general nomenclature of cleavage site positions designates the cleavage site between P1-P1′, incrementing the position number in the N-terminal direction of the cleaved peptide bond (P2, P3, P4, etc. . . . ) and incrementing position number in the C-terminal direction in the same manner (P2′, P3′, P4′ etc. . . . ). In some cases, a protease cleavage site may include one to six amino acid residues on either side of the scissile bond, which bind to the active site of the protease and are used for recognition as a substrate, having an amino acid sequence that may be cleaved by a protease, such as, for example, a matrix metalloproteinase or a furin. Examples of such sites include Gly-Pro-Leu-Gly-Ile-Ala-Gly-Gln or Ala-Val-Arg-Trp-Leu-Leu-Thr-Ala, which can be cleaved by metalloproteinases, and Arg-Arg-Arg-Arg-Arg-Arg, which is cleaved by a furin. In therapeutic applications, the protease cleavage site can be cleaved by a protease that is produced by target cells, for example cancer cells or infected cells, or pathogens.

As used herein, “protein” and “polypeptide” may be used interchangeably and collectively refer to any polymer of two or more amino acids linked by peptide bonds and does not refer to a specific length of the product. Thus, “peptides,” “protein,” “amino acid chain,” or any other term used to refer to a chain of two or more amino acids, are included within the definition of “polypeptide,” and the term “polypeptide” may be used instead of, or interchangeably with, any of these terms. The term “polypeptide” is also intended to include products of post-translational modifications of the polypeptide like, e.g., glycosylation, which are well known in the art.

As used herein, “protease,” “proteinase,” “peptidase,” and “proteolytic enzyme” may be used interchangeably and collectively refer to an enzyme which catalyzes proteolysis, such as by hydrolyzing the peptide bonds of a protein.

As used herein, “protease substrate motif” refers to a polypeptide motif comprising a protease cleavage site.

As used herein, “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups may include, but are not limited to, benzyloxycarbonyl; 9-fluorenylmethyloxycarbonyl (Fmoc); tert-butyloxycarbonyl (Boc); allyloxycarbonyl (Alloc); p-toluene sulfonyl (Tos); 2,2,5,7,8-pentamethylchroman-6-sulfonyl (Pmc); 2,2,4,6,7-pentamethyl-2,3-dihydrobenzofuran-5-sulfonyl (Pbf); mesityl-2-sulfonyl (Mts); 4-methoxy-2,3,6-trimethylphenylsulfonyl (Mtr); acetamido; phthalimido; and the like. Other protecting groups are known to those of skill in the art including, for example, those described by Green and Wuts (Protective Groups in Organic Synthesis, 4th Ed. 2007, Wiley-Interscience, New York).

As used herein, “PubChem CID” refers to a compound ID number used as a database identifier from “PubChem,” a chemical information database administrated by the U.S. National Library of Medicine (National Center for Biotechnological Information, U.S. National Library of Medicine, 8600 Rockville Pike, Bethesda, MD 20894, USA).

As used herein, “residue” refers to single molecular unit within a polymer. For example, a residue may include, respectively, a single amino acid within a polypeptide or a single nucleotide within a polynucleotide.

As used herein, “reversible bond” refers to a chemical bond having an activation energy sufficiently low enough to react in a context.

As used herein, “scissile bond” refers to a covalent bond that can be broken by an enzyme, such as a peptide bond cleaved by a protease.

As used herein, “self-assembling polypeptide” refers to a polypeptide comprising a polypeptide motif that is configured to self-assemble.

As used herein, “self-assembly” is a process in which a disordered system of pre-existing components forms an organized structure or pattern as a consequence of specific, local interactions between the components themselves. For example, as disclosed herein, β-strand motifs dissociated by protease cleavage may form a β-sheet structure as a consequence of the local hydrogen bonding interactions between the β-strand motifs themselves.

As used herein, “sequence identity” refers to the similarity between two nucleic acid sequences, or two amino acid sequences. Sequence identity is frequently measured in terms of percent identity (or similarity or homology); the higher the percentage, the more similar the two sequences are. Polypeptides or domains thereof that have a significant amount of sequence identity and function the same or similarly to one another—for example, the same protein in different species—can be called “homologs.” Methods of alignment are well known in the art. Various programs and alignment algorithms are described in: Smith & Waterman, Adv. Appl. Math. 2: 482, 1981; Needleman & Wunsch, J. Mol. Biol. 48: 443, 1970; Pearson & Lipman, Proc. Natl. Acad. Sci. USA 85: 2444, 1988; Higgins & Sharp, Gene, 73: 237-244, 1988; Higgins & Sharp, Comput. Appl. Biosci. 5: 151-153, 1989; Corpet et al., Nucl. Acids Res. 16, 10881-90, 1988; Huang et al., Comput. Appl. Biosci. 8, 155-65, 1992; and Pearson, Methods Mol. Biol. 24:307-331, 1994. Altschul et al. (J. Mol. Biol. 215:403-410, 1990) presents a detailed consideration of sequence alignment methods and homology calculations. The NCBI Basic Local Alignment Search Tool (BLAST) is available from several sources, including the National Center for Biotechnology Information (NCBI, Bethesda, MD) and on the Internet, for use in connection with the sequence analysis programs blastp, blastn, blastx, tblastn and tblastx. In a further example, methods for determining the extent of an amino acid sequence identity of an arbitrary polypeptide relative to the amino acid sequence, the SIM Local similarity program may be employed (Huang and Webb Miller (1991), Advances in Applied Mathematics, 12: 337-357), that is freely available. For multiple alignment analysis, ClustalW can be used (Thompson et al. (1994) Nucleic Acids Res., 22: 4673-4680). Nucleic acid sequences that do not show a high degree of sequence identity may nevertheless encode similar amino acid sequences, due to the degeneracy of the genetic code. Skilled persons will understand that changes in nucleic acid sequence can be made using this degeneracy to produce multiple nucleic acid molecules that all encode substantially the same protein.

As used herein, “sequence” refers to a particular order in which things follow each other, such as the order of repeating molecular units in a polymer. For example, skilled persons will understand that the order of nucleic acid sequences and amino acid sequences are referred to by convention in the order of, respectively, nucleic acid residues running from a 5′ end to a 3′ end and amino acid residues running from a N-terminus to a C-terminus.

As used herein, “substrate” refers to a molecule or material that is acted upon by another molecule or material, such as by an enzyme.

As used herein, “trigger” refers to the immediate cause eliciting an effect, such as a change in configuration or an activation.

As used herein, “to bind” and its verb conjugates refer to the reversible or non-reversible attachment of one molecule to another.

As used herein, “to dissociate the β-strand motif” refers to the β-strand motif being cleaved from a self-assembling polypeptide at the scissile bond of the cleaving protease.

As used herein, “to specifically hybridize with a protease” refers to a protease substrate motif having a protease cleavage site that acts as substrate for a specific protease. Skilled persons will understand that one criteria for distinguishing one protease from another is its action upon substrates and that curated databases of known protease cleavage sites in substrates are readily available. For example, the MEROPS database is a curated protease repository known in the art that catalogs and identifies the proteolytic activity corresponding to specific protease-substrate interactions (Rawlings, N. D. et al., The MEROPS database of proteolytic enzymes, their substrates and inhibitors in 2017 and a comparison with peptidases in the PANTHER database. Nucleic Acids Res. 46, D624-D632 (2018); accessible at: ebi.ac.uk/merops/). As used herein, “MEROPS ID:” refers to a MEROPS database identifier. Moreover, skilled persons will understand that many methods exist for identifying specific protease-substrate relationships (Uliana et al., Mapping specificity, cleavage entropy, allosteric changes and substrates of blood proteases in a high-throughput screen, Nature Communications, 12:1693 (2021); which is hereby incorporated by reference in its entirety). Curated proteolytic databases known in the art may include the MEROPS database (accessible at: ebi.ac.uk/merops/), the PANTHER database (accessible at: pantherdb.org), the BRENDA database (accessible at: brenda-enzymes.org), the TopFIND database (accessible at: topfind.clip.msl.ubc.ca), and the UniProt database (accessible at: uniprot.org).

In an exemplary embodiment, the disclosed materials and methods relate to the detection of proteases in an aqueous milieu through utilizing enzyme-instructed self-assembly (EISA) of self-assembling polypeptides. In the exemplary embodiment, a self-assembling polypeptide comprises a β-strand motif configured to self-assemble with one or more nominally identical β-strand motifs and form an anti-parallel β-sheet structure. The β-strand motif is operatively connected to a hydrophilic motif by a protease substrate motif that comprises a protease cleavage site configured to specifically hybridize with a protease. Whereby, when in an aqueous milieu and upon hybridization of the protease to the protease cleavage site, the protease cleaves the self-assembling polypeptide and dissociates the β-strand motif, allowing the dissociated β-strand motif to self-assemble with the one or more nominally identical β-strand motifs and thereby form the anti-parallel β-sheet structure.

In some embodiments, the β-strand motif comprises, from N-terminus to C-terminus, an amino acid sequence selected from any one of: Fmoc-Phe-Lys-Phe-Glu (SEQ ID NO: 1), Fmoc-Phe-Phe (SEQ ID NO: 2), Fmoc-Phe-Phe-(D-Lys)-(D-Lys) (SEQ ID NO: 3), Fmoc-Phe-(D-Lys)-Phe-(D-Lys) (SEQ ID NO: 4), and Phe-Glu-Phe-Glu-Phe-Lys-Phe-Lys (SEQ ID NO: 5), Phe-Glu-Phe-Lys-Phe-Glu-Phe-Lys (SEQ ID NO: 6), Phe-Lys-Phe-Glu-Phe-Lys-Phe-Glu (SEQ ID NO: 7), (D-Phe)-(D-Lys)-(D-Phe)-(D-Glu)-(D-Phe)-(D-Lys)-(D-Phe)-(D-Glu) (SEQ ID NO: 8), Acetyl-Phe-Lys-Phe-Glu-Phe-Lys-Phe-Glu-Amide (SEQ ID NO: 9), and Acetyl-Phe-Lys-Phe-Glu-Phe-Lys-Phe-Amide (SEQ ID NO: 10).

In some embodiments, the net charge of the hydrophilic motif is negative. In some embodiments, the hydrophilic motif comprises a zwitterion.

In some embodiments, the hydrophilic motif comprises, from N-terminus to C-terminus, an amino acid sequence selected from any one of: Glu-Glu-Glu-Gly-Ser-Gly-Glu-Glu-Glu (SEQ ID NO: 11), Asp-Asp-Asp-Gly-Ser-Gly-Glu-Glu-Glu (SEQ ID NO: 12), Glu-Glu-Glu-Gly-Ser-Gly-Asp-Asp-Asp (SEQ ID NO: 13), Glu-Glu-Glu-Gly-Ser-Gly-Glu-Glu-Glu-Gly-Ser-Gly-Glu-Glu-Glu (SEQ ID NO: 14), Asp-Asp-Asp-Gly-Ser-Gly-Glu-Glu-Glu-Gly-Ser-Gly-Glu-Glu-Glu (SEQ ID NO: 15), Glu-Glu-Glu-Gly-Ser-Gly-Asp-Asp-Asp-Gly-Ser-Gly-Glu-Glu-Glu (SEQ ID NO: 16), Asp-Asp-Asp-Gly-Ser-Gly-Asp-Asp-Asp-Gly-Ser-Gly-Asp-Asp-Asp (SEQ ID NO: 17), Asp-Asp-Gly-Asp-Asp (SEQ ID NO: 18), Asp-Asp-Gly-Asp-Asp-Gly-Asp-Asp (SEQ ID NO: 19), Asp-Asp-Gly-Asp-Asp-Gly-Asp-Asp-Gly-Asp-Asp (SEQ ID NO: 20), Asp-Asp-Gly-Asp-Asp-Gly-Asp-Asp-Gly-Asp-Asp-Gly-Asp-Asp (SEQ ID NO: 21), Glu-Glu-Gly-Glu-Glu (SEQ ID NO: 22), Glu-Glu-Gly-Glu-Glu-Gly-Glu-Glu (SEQ ID NO: 23), Glu-Glu-Gly-Glu-Glu-Gly-Glu-Glu-Gly-Glu-Glu (SEQ ID NO: 24), Glu-Glu-Gly-Glu-Glu-Gly-Glu-Glu-Gly-Glu-Glu-Gly-Glu-Glu (SEQ ID NO: 25), Glu-Glu-Gly-Lys-Asp-Asp-Gly-Glu-Glu-Gly-Asp-Asp (SEQ ID NO: 26), Asp-Asp-Gly-Glu-Glu-Gly-Asp-Asp-Gly-Glu-Glu (SEQ ID NO: 27), Glu-Glu-Gly-Lys-Lys-Gly-Glu-Glu-Gly-Lys-Lys-Gly-Glu-Glu (SEQ ID NO: 28), and Asp-Asp-Gly-Glu-Glu-Gly-Lys-Lys-Gly-Glu-Glu-Gly-Lys-Lys (SEQ ID NO: 29), Asp-Ser-Asp-Ser (SEQ ID NO: 30), Asp-Ser-Asp-Ser-Asp-Ser (SEQ ID NO: 31), Asp-Ser-Asp-Ser-Asp-Ser-Asp-Ser (SEQ ID NO: 32), Asp-Ser-Asp-Ser-Asp-Ser-Asp-Ser-Asp-Ser (SEQ ID NO: 33), Asp-Ser-Asp-Ser-Asp-Ser-Asp-Ser-Asp-Ser-Asp-Ser (SEQ ID NO: 34), Asp-Ser-Asp-Ser-Asp-Ser-Asp-Ser-Asp-Ser-Asp-Ser-Asp-Ser (SEQ ID NO: 35), Asp-Ser-Asp-Ser-Asp-Ser-Asp-Ser-Asp-Ser-Asp-Ser-Asp-Ser-Asp-Ser (SEQ ID NO: 36), Glu-Ser-Glu-Ser (SEQ ID NO: 37), Glu-Ser-Glu-Ser-Glu-Ser (SEQ ID NO: 38), Glu-Ser-Glu-Ser-Glu-Ser-Glu-Ser (SEQ ID NO: 39), Glu-Ser-Glu-Ser-Glu-Ser-Glu-Ser-Glu-Ser (SEQ ID NO: 40), Glu-Ser-Glu-Ser-Glu-Ser-Glu-Ser-Glu-Ser-Glu-Ser (SEQ ID NO: 41), Glu-Ser-Glu-Ser-Glu-Ser-Glu-Ser-Glu-Ser-Glu-Ser-Glu-Ser (SEQ ID NO: 42), Glu-Ser-Glu-Ser-Glu-Ser-Glu-Ser-Glu-Ser-Glu-Ser-Glu-Ser-Glu-Ser (SEQ ID NO: 43), Glu-Glu (SEQ ID NO: 44), Glu-Glu-Glu (SEQ ID NO: 45), Glu-Glu-Glu-Glu (SEQ ID NO: 46), Glu-Glu-Glu-Glu-Glu (SEQ ID NO: 47), Glu-Glu-Glu-Glu-Glu-Glu (SEQ ID NO: 48), Glu-Glu-Glu-Glu-Glu-Glu-Glu (SEQ ID NO: 49), Glu-Glu-Glu-Glu-Glu-Glu-Glu-Glu (SEQ ID NO: 50), Glu-Glu-Glu-Glu-Glu-Glu-Glu-Glu-Glu (SEQ ID NO: 51), Glu-Glu-Glu-Glu-Glu-Glu-Glu-Glu-Glu-Glu (SEQ ID NO: 52), Asp-Asp (SEQ ID NO: 53), Asp-Asp-Asp (SEQ ID NO: 54), Asp-Asp-Asp-Asp (SEQ ID NO: 55), Asp-Asp-Asp-Asp-Asp (SEQ ID NO: 56), Asp-Asp-Asp-Asp-Asp-Asp (SEQ ID NO: 57), Asp-Asp-Asp-Asp-Asp-Asp-Asp (SEQ ID NO: 58), Asp-Asp-Asp-Asp-Asp-Asp-Asp-Asp (SEQ ID NO: 59), Asp-Asp-Asp-Asp-Asp-Asp-Asp-Asp-Asp (SEQ ID NO: 60), Asp-Asp-Asp-Asp-Asp-Asp-Asp-Asp-Asp-Asp (SEQ ID NO: 61), Glu-Asp (SEQ ID NO: 62), Glu-Asp-Glu-Asp (SEQ ID NO: 63), Glu-Asp-Glu-Asp-Glu-Asp (SEQ ID NO: 64), Glu-Asp-Glu-Asp-Glu-Asp-Glu-Asp (SEQ ID NO: 65), Glu-Asp-Glu-Asp-Glu-Asp-Glu-Asp-Glu-Asp (SEQ ID NO: 66), Asp-Glu (SEQ ID NO: 67), Asp-Glu-Asp-Glu (SEQ ID NO: 68), Asp-Glu-Asp-Glu-Asp-Glu (SEQ ID NO: 69), Asp-Glu-Asp-Glu-Asp-Glu-Asp-Glu (SEQ ID NO: 70), Asp-Glu-Asp-Glu-Asp-Glu-Asp-Glu-Asp-Glu (SEQ ID NO: 71), and pSer-pSer-Gly-Ser-Gly-pSer-pSer (SEQ ID NO: 72).

In some embodiments, the hydrophilic motif comprises, from N-terminus to C-terminus, an amino acid sequence selected from any one of: Lys-Glu-Lys-Glu-Lys (SEQ ID NO: 73), Lys-Glu-Lys-Glu-Lys-Glu-Lys (SEQ ID NO: 74), Lys-Glu-Lys-Glu-Lys-Glu-Lys-Glu-Lys (SEQ ID NO: 75), Lys-Glu-Lys-Glu-Lys-Glu-Lys-Glu-Lys-Glu-Lys (SEQ ID NO: 76), Arg-Asp-Arg-Asp-Arg (SEQ ID NO: 77), Arg-Asp-Arg-Asp-Arg-Asp-Arg (SEQ ID NO: 78), Arg-Asp-Arg-Asp-Arg-Asp-Arg-Asp-Arg (SEQ ID NO: 79), Arg-Asp-Arg-Asp-Asp-Arg-Asp-Arg-Asp-Arg-Arg (SEQ ID NO: 80), Arg-Glu-Arg-Glu-Arg (SEQ ID NO: 81), Arg-Glu-Arg-Glu-Arg-Glu-Arg (SEQ ID NO: 82), Arg-Glu-Arg-Glu-Arg-Glu-Arg-Glu-Arg (SEQ ID NO: 83), Arg-Glu-Arg-Glu-Arg-Glu-Arg-Glu-Arg-Glu-Arg (SEQ ID NO: 84), Lys-Asp-Lys-Asp-Lys (SEQ ID NO: 85), Lys-Asp-Lys-Asp-Lys-Asp-Lys (SEQ ID NO: 86), Lys-Asp-Lys-Asp-Lys-Asp-Lys (SEQ ID NO: 87), Lys-Asp-Lys-Asp-Lys-Asp-Lys (SEQ ID NO: 88), pSer-Lys-pSer-Lys-Lys (SEQ ID NO: 89), pSer-Lys-pSer-Lys-pSer-Lys-Lys (SEQ ID NO: 90), pSer-Lys-pSer-Lys-pSer-Lys-pSer-Lys-Lys (SEQ ID NO: 91), pSer-Lys-pSer-Lys-pSer-Lys-pSer-Lys-pSer-Lys-Lys (SEQ ID NO: 92), pSer-Arg-pSer-Arg-Arg (SEQ ID NO: 93), pSer-Arg-pSer-Arg-pSer-Arg-Arg (SEQ ID NO: 94), pSer-Arg-pSer-Arg-pSer-Arg-pSer-Arg-Arg (SEQ ID NO: 95), pSer-Arg-pSer-Arg-pSer-Arg-pSer-Arg-pSer-Arg-Arg (SEQ ID NO: 96), Ser-Lys-Asp-Ser-Lys-Asp-Lys (SEQ ID NO: 97), Ser-Lys-Asp-Ser-Lys-Asp-Ser-Lys-Asp-Lys (SEQ ID NO: 98), Ser-Lys-Asp-Ser-Lys-Asp-Ser-Lys-Asp-Ser-Lys-Asp-Lys (SEQ ID NO: 99), Ser-Arg-Glu-Ser-Arg-Glu-Arg (SEQ ID NO: 100), Ser-Arg-Glu-Ser-Arg-Glu-Ser-Arg-Glu-Arg (SEQ ID NO: 101), Ser-Arg-Glu-Ser-Arg-Glu-Ser-Arg-Glu-Ser-Arg-Glu-Arg (SEQ ID NO: 102), Ser-Lys-Glu-Ser-Lys-Glu-Lys (SEQ ID NO: 103), Ser-Lys-Glu-Ser-Lys-Glu-Ser-Lys-Glu-Lys (SEQ ID NO: 104), Ser-Lys-Glu-Ser-Lys-Glu-Ser-Lys-Glu-Ser-Lys-Glu-Lys (SEQ ID NO: 105), Ser-Arg-Asp-Ser-Arg-Asp-Arg (SEQ ID NO: 106), Ser-Arg-Asp-Ser-Arg-Asp-Ser-Arg-Asp-Arg (SEQ ID NO: 107), and Ser-Arg-Asp-Ser-Arg-Asp-Ser-Arg-Asp-Ser-Arg-Asp-Arg (SEQ ID NO: 108).

In some embodiments, the hydrophilic motif comprises, from N-terminus to C-terminus, an amino acid sequence selected from any one of: Lys-Glu-Lys-Glu (SEQ ID NO: 109), Lys-Glu-Lys-Glu-Lys-Glu (SEQ ID NO: 110), Lys-Glu-Lys-Glu-Lys-Glu-Lys-Glu (SEQ ID NO: 111), Lys-Glu-Lys-Glu-Lys-Glu-Lys-Glu-Lys-Glu (SEQ ID NO: 112), Arg-Asp-Arg-Asp (SEQ ID NO: 113), Arg-Asp-Arg-Asp-Arg-Asp (SEQ ID NO: 114), Arg-Asp-Arg-Asp-Arg-Asp-Arg-Asp (SEQ ID NO: 115), Arg-Asp-Arg-Asp-Asp-Arg-Asp-Arg-Asp-Arg (SEQ ID NO: 116), Arg-Glu-Arg-Glu (SEQ ID NO: 117), Arg-Glu-Arg-Glu-Arg-Glu (SEQ ID NO: 118), Arg-Glu-Arg-Glu-Arg-Glu-Arg-Glu (SEQ ID NO: 119), Arg-Glu-Arg-Glu-Arg-Glu-Arg-Glu-Arg-Glu (SEQ ID NO: 120), Lys-Asp-Lys-Asp (SEQ ID NO: 121), Lys-Asp-Lys-Asp-Lys-Asp (SEQ ID NO: 122), Lys-Asp-Lys-Asp-Lys-Asp (SEQ ID NO: 123), Lys-Asp-Lys-Asp-Lys-Asp (SEQ ID NO: 124), pSer-Lys-pSer-Lys (SEQ ID NO: 125), pSer-Lys-pSer-Lys-pSer-Lys (SEQ ID NO: 126), pSer-Lys-pSer-Lys-pSer-Lys-pSer-Lys (SEQ ID NO: 127), pSer-Lys-pSer-Lys-pSer-Lys-pSer-Lys-pSer-Lys (SEQ ID NO: 128), pSer-Arg-pSer-Arg (SEQ ID NO: 129), pSer-Arg-pSer-Arg-pSer-Arg (SEQ ID NO: 130), pSer-Arg-pSer-Arg-pSer-Arg-pSer-Arg (SEQ ID NO: 131), pSer-Arg-pSer-Arg-pSer-Arg-pSer-Arg-pSer-Arg (SEQ ID NO: 132), Ser-Lys-Asp-Ser-Lys-Asp (SEQ ID NO: 133), Ser-Lys-Asp-Ser-Lys-Asp-Ser-Lys-Asp (SEQ ID NO: 134), Ser-Lys-Asp-Ser-Lys-Asp-Ser-Lys-Asp-Ser-Lys-Asp (SEQ ID NO: 135), Ser-Arg-Glu-Ser-Arg-Glu (SEQ ID NO: 136), Ser-Arg-Glu-Ser-Arg-Glu-Ser-Arg-Glu (SEQ ID NO: 137), Ser-Arg-Glu-Ser-Arg-Glu-Ser-Arg-Glu-Ser-Arg-Glu (SEQ ID NO: 138), Ser-Lys-Glu-Ser-Lys-Glu (SEQ ID NO: 139), Ser-Lys-Glu-Ser-Lys-Glu-Ser-Lys-Glu (SEQ ID NO: 140), Ser-Lys-Glu-Ser-Lys-Glu-Ser-Lys-Glu-Ser-Lys-Glu (SEQ ID NO: 141), Ser-Arg-Asp-Ser-Arg-Asp (SEQ ID NO: 142), Ser-Arg-Asp-Ser-Arg-Asp-Ser-Arg-Asp (SEQ ID NO: 143), and Ser-Arg-Asp-Ser-Arg-Asp-Ser-Arg-Asp-Ser-Arg-Asp (SEQ ID NO: 144), in which the C-terminus of the selected amino acid sequence is amidated.

Skilled persons will understand that C-terminal amidation of an amino acid residue may be useful for providing an uncharged polypeptide terminus, enhancing the solubility of the polypeptide in an aqueous milieu, or increasing the polypeptide's resistance to enzymatic degradation by aminopeptidases, exopeptidases, and synthetases (Arispe N., et al., Efficiency of Histidine-Associating Compounds for Blocking the Alzheimer's AB Channel Activity and Cytotoxicity. Biophysical Journal Vol. 95:4879-4889 (2008)).

In some embodiments, the protease substrate motif comprises an amino acid sequence selected from any one of: Ala-Ala-Asn-Gly (SEQ ID NO: 145), Leu-Ala-Gly-Gly-Ala-Gly (SEQ ID NO: 146), Arg-Ser-Lys-Arg-Val-Ser-Gly (SEQ ID NO: 147), Arg-Ser-Lys-Arg-Ser (SEQ ID NO: 148), Ser-Ala-Gin-Ala-Val-Val-Ser-Gin (SEQ ID NO: 149), Ala-Gin-Ala-Val-Val-Ser (SEQ ID NO: 150), Leu-Ala-Gln-Ala-Val-Val-Ser-Ala (SEQ ID NO: 151), Ala-Gin-Ala-Val-Val-Ser (SEQ ID NO: 152), Leu-Ala-Ala-Ala-Val-Val-Ser-Ser (SEQ ID NO: 153), Ala-Ala-Ala-Val-Val (SEQ ID NO: 154), Pro-Ala-Ala-Ala-Gln-Arg-Leu-Arg (SEQ ID NO: 155), Ala-Ala-Ala-Gln-Arg-Leu (SEQ ID NO: 156), Leu-Pro-Ala-Ala-Leu-Val-Gly-Ala (SEQ ID NO: 157), Pro-Ala-Ala-Leu (SEQ ID NO: 158), Leu-Pro-Ser-Gly-Leu-Val-Gly-Ala (SEQ ID NO: 159), Pro-Ser-Gly-Leu (SEQ ID NO: 160), Gly-Pro-Ala-Gly-Leu-Ala-Gly-Ala (SEQ ID NO: 161), Pro-Ala-Gly-Leu (SEQ ID NO: 162), Gly-Pro-Gly-Gly-Leu-Ala-Gly-Ala (SEQ ID NO: 163), Gly-Pro-Leu-Gly-Leu-Val-Gly-Gln (SEQ ID NO: 164), Pro-Leu-Gly-Leu (SEQ ID NO: 165), Gly-Pro-Ala-Gly-Leu-Gly-Gly-Gly (SEQ ID NO: 166), Pro-Ala-Gly-Leu (SEQ ID NO: 167), Gly-Pro-Pro-Gly-Leu-Arg-Gly-Pro (SEQ ID NO: 168), Pro-Pro-Gly-Leu (SEQ ID NO: 169), Gly-Pro-Leu-Gly-Leu-Arg-Gly-Pro (SEQ ID NO: 170), Pro-Leu-Gly-Leu (SEQ ID NO: 171), Gly-Pro-Ala-Gly-Leu-Arg-Thr-Glu (SEQ ID NO: 172), Leu-Pro-Gln-Gly-Leu-Ala-Gly-Arg (SEQ ID NO: 173), Pro-Ala-Gly-Leu (SEQ ID NO: 174), Glu-Ala-Glu-Asn-Gly-Glu-Leu-Pro (SEQ ID NO: 175), Ala-Ala-Asn-Gly (SEQ ID NO: 176), Asp-Asn-Phe-Leu-Val (SEQ ID NO: 177), Asp-Asn-Phe-Phe-Val (SEQ ID NO: 178), Gly-Leu-Ala-Gly-Gly-Ala-Gly-Gly (SEQ ID NO: 179), Leu-Ala-Gly-Gly-Ala-Gly (SEQ ID NO: 180), Gly-Leu-Val-Ala-Leu-Leu-Ala-Gly-Gly (SEQ ID NO: 181), Leu-Glu-Val-Leu-Ile-Val (SEQ ID NO: 182), Glu-Val-Leu-Ile-Val (SEQ ID NO: 183), Glu-Val-Val-Leu-Val-Ala-Leu-Ala (SEQ ID NO: 184), Glu-Val-Val-Phe-Val-Ala-Leu-Ala (SEQ ID NO: 185), Val-Leu-Val-Ala (SEQ ID NO: 186), Val-Phe-Val-Ala (SEQ ID NO: 187), Asp-Val-Leu-Leu-Ser-Trp-Ala-Val (SEQ ID NO: 188), Val-Leu-Leu-Ser-Trp (SEQ ID NO: 189), Ala-Lys-Leu-Lys-Glu-Glu-Asp-Asp (SEQ ID NO: 190), Ala-Gly-Leu-Gly-Glu-Glu-Asp-Asp (SEQ ID NO: 191), Ala-Leu-Leu-Gly-Ala-Pro-Pro-Pro (SEQ ID NO: 192), Gly-Leu-Leu-Gly-Ser-Glu-Pro-Glu (SEQ ID NO: 193), Leu-Gly-Ala-Pro (SEQ ID NO: 194), Leu-Gly-Ser-Glu (SEQ ID NO: 195), Ala-Ala-Lys-Gly-Ala-Ala-Pro-Glu (SEQ ID NO: 196), Leu-Gly-Ala-Ala (SEQ ID NO: 197), Ser-Ser-Gln-Tyr-Ser-Ser-Asn-Gly (SEQ ID NO: 198), Ser-Gln-Gln-Tyr-Ser-Ser-Asn-Gly (SEQ ID NO: 199), Ser-Ser-Gln-Gln-Ser-Ser-Asn-Gly (SEQ ID NO: 200), Gly-Gly-Ser-Arg-Ser-Gly-Gly-Gly (SEQ ID NO: 201), Gly-Gly-Ser-Arg-Ser-Pro-Gly-Gly (SEQ ID NO: 202), Gly-Val-Asn-Leu-Asp-Val-Glu-Val (SEQ ID NO: 203), Arg-Gln-Ala-Arg-Lys-Val-Gly-Gly (SEQ ID NO: 204), and Ala-Ala-Ala-Arg-Lys-Val-Gly-Gly (SEQ ID NO: 205).

In some embodiments, the protease substrate motif is configured as a substrate of Furin proteases (also known by skilled persons as paired basic amino acid cleaving enzyme (PACE). PACE is a serine protease having substrates that include the amino acid sequences SEQ ID NO: 147 and SEQ ID NO: 148 (see MEROPS ID: S08.071). Skilled persons will understand that Furin overexpression is a prognostic marker in various cancers including cervical, brain, lung, stomach, and bile duct cancer (Zhou B. and Gao S., Pan-Cancer Analysis of FURIN as a Potential Prognostic and Immunological Biomarker, Front. Mol. Biosci. 8:648402. Doi: 10.3389/fmolb.2021.648402, (2021)).

In some embodiments, the protease substrate motif is configured as a substrate of disintegrin and metalloproteases (ADAMs). ADAMs are a family or proteolytic enzymes that are known by skilled persons to be biomarkers and therapeutic targets for cancer (Duffy, M. J., Mullooly, M., O'Donovan, N. et al. The ADAMs family of proteases: new biomarkers and therapeutic targets for cancer?. Clin. Proteom. 8, 9 (2011); Mullooly, M. et al., The ADAMs family of proteases as targets for the treatment of cancer. Cancer Biol. and Therapy. 17:8 (2016)). For example, in some embodiments, ADAM10 (also known by skilled persons as alpha-secretase) is a metalloproteinase having substrates that include the amino acid sequences SEQ ID NO: 149 and SEQ ID NO: 150 (see MEROPS ID: M12.210). Skilled persons will understand that ADAM10 is protective against amyloid plaques in Alzheimer's Disease and is elevated in a variety of cancers including liver, skin, gastric, lung, pancreatic, and bladder cancer (Yuan, Q., Yu, H., Chen, J. et al. ADAM10 promotes cell growth, migration, and invasion in osteosarcoma via regulating E-cadherin/β-catenin signaling pathway and is regulated by miR-122-5p. Cancer Cell Int. 20, 99 (2020)). In a further embodiment, ADAM17 (also known as tumor-necrosis factor alpha converting enzyme (TACE)). TACE is a metalloproteinase having substrates that include SEQ ID NO: 151, SEQ ID NO: 152, SEQ ID NO: 153, and SEQ ID NO: 154 (see MEROPS ID: M12.217). Skilled persons will understand that ADAM 17 is elevated in various cancers including breast and lung cancer. In a still further embodiment, ADAM8 is a metalloproteinase having substrates that include SEQ ID NO: 155 and SEQ ID NO: 156 (see MEROPS ID: M12.208). Skilled persons will understand that ADAM 8 is elevated in various cancers including lung, pancreatic, liver, prostate, kidney, brain, and colorectal cancer.

In some embodiments, the protease substrate motif is configured as a substrate of matrix metalloproteinases (MMPs). MMPs (also known as matrix metallopeptidases) are known by skilled persons as biomarkers for various diseases including cancer, cardiovascular disease, and arthritis (Page-McCaw, A. et al., Matrix metalloproteinases and the regulation of tissue remodeling. Nature Reviews vol. 8, 221-233 (2007); Quintero-Fabián S et al., Role of Matrix Metalloproteinases in Angiogenesis and Cancer. Front. Oncol. 9:1370 (2019); Park K. C. et al., The Role of Extracellular Proteases in Tumor Progression and the Development of Innovative Metal Ion Chelators That Inhibit Their Activity, Int. J. Mol. Sci., 21(18), 6805 (2020); Eckhard U., et al., Active site specificity profiling of the matrix metalloproteinase family: Proteomic identification of 4300 cleavage sites by nine MMPs explored with structural and synthetic peptide cleavage analyses. Matrix Biol. 49, 37-60 (2016)). For example, in some embodiments, MMP-2 (also known as gelatinase A) is a metalloprotease with substrates that include SEQ ID NO: 157, SEQ ID NO: 158, SEQ ID NO: 159, and SEQ ID NO: 160 (see MEROPS ID: M10.003). Skilled persons will understand that MMP-2 is elevated in acute coronary disease, atherosclerosis, arthritis, and in a variety of cancers including brain, ovarian, pancreatic, and bladder cancer. In a further embodiment, MMP-9 (also known as gelatinase B) is a metalloprotease having substrates that include SEQ ID NO: 161, SEQ ID NO: 162, and SEQ ID NO: 163 (see MEROPS ID: M10.004). Skilled persons will understand that MMP-9 is elevated in acute coronary disease, atherosclerosis, arthritis and in a variety of cancers including breast, pancreatic, bladder, colorectal, gastric, prostate, and brain cancer. In a still further embodiment, MMP-1 (also known as collagenase 1) is a metalloprotease having substrates that include SEQ ID NO: 164 and SEQ ID NO: 165 (see MEROPS ID: M10.001). Skilled persons will understand that MMP-1 is elevated in acute coronary syndrome, arthritis, pre-cancerous breast hyperplasia, and in cancers including lung and colorectal cancer. In a yet further embodiment, MMP-7 (also known as matrilysin) is a metalloprotease having substrates that include SEQ ID NO: 166 and SEQ ID NO: 167 (see MEROPS ID: M10.008). Skilled persons will understand that MMP-7 is elevated in a variety of cancers including pancreatic, lung, and colorectal cancer. In a yet further embodiment, MMP-13 (also known as collagenase 3) is a metalloprotease having substrates that include SEQ ID NO: 168, SEQ ID NO: 169, SEQ ID NO: 170, and SEQ ID NO: 171 (see MEROPS ID: M10.013). Skilled persons will understand that MMP-13 is elevated in arthritis and in cancers including breast and colorectal cancer. In a yet further embodiment, MMP-14 (also known as membrane-type matrix metalloproteinase-1) is a metalloprotease having substrates that include SEQ ID NO: 172, SEQ ID NO: 173, and SEQ ID NO: 174 (see MEROPS ID: M10.014).

In some embodiments, the protease substrate motif is configured as a substrate of legumain (LGMN) (also known as asparagine endopeptidase). LGMN is a metalloprotease having substrates that include SEQ ID NO: 175 and SEQ ID NO: 176 (see MEROPS ID: C13.004). Skilled persons will understand that LGMN is elevated in a variety of cancers including breast, colon, lung, prostate, ovarian, and brain cancer (Liu C. et al. Overexpression of legumain in tumors is significant for invasion/metastasis and a candidate enzymatic target for prodrug therapy. Cancer Res. June 1; 63(11):2957-64 (2003)).

In some embodiments, the protease substrate motif is configured as a substrate of Cathepsins. Cathepsins are known by skilled persons to be overexpressed in various cancers and are in some cases associated with tumor metastasis (Tan G. J., Cathepsins mediate tumor metastasis. World J Biol Chem November 26; 4(4): 91-101 (2013)). In some embodiments, Cathepsin A is a serine protease having substrates that include SEQ ID NO: 177 and SEQ ID NO: 178 (see MEROPS ID: S10.002). Skilled persons will understand that Cathepsin A is elevated in melanoma. In a further embodiment, Cathepsin B is a serine protease having substrates that include SEQ ID NO: 179, SEQ ID NO: 180, and SEQ ID NO: 181 (see MEROPS ID: C01.060). Skilled persons will understand that Cathepsin B is elevated in various cancers including breast, skin, link, colon, cervical, brain, and liver cancer. In a still further embodiment, Cathepsin D is an aspartic acid protease having substrates that include SEQ ID NO: 182 and SEQ ID NO: 183 (see MEROPS ID: A01.009). Skilled persons will understand that Cathepsin D is elevated in a broad range of cancers including thyroid, brain, breast, and lung cancer. In a yet further embodiment, Cathepsin E is an aspartic acid protease with substrates that include SEQ ID NO: 184, SEQ ID NO: 185, SEQ ID NO: 186, and SEQ ID NO: 187 (see MEROPS ID: A01.010). Skilled persons will understand that Cathepsin E is elevated in pancreatic and gastric cancers. In a yet further embodiment, Cathepsin G is a serine protease with substrates that include SEQ ID NO: 188 and SEQ ID NO: 189 (see MEROPS ID: SO1.133). Skilled persons will understand that Cathepsin G is elevated in breast cancer. In a yet further embodiment, Cathepsin K (CTSK) is a cysteine protease having substrates that include SEQ ID NO: 190 and SEQ ID NO: 191 (see MEROPS ID: C01.036). Skilled persons will understand that CTSK is elevated various cancers including breast cancer and glioblastoma and is also involved in the disease progression of osteoporosis and osteoarthritis (Duong L. T. et al., Efficacy of a Cathepsin K Inhibitor in a Preclinical Model for Prevention and Treatment of Breast Cancer Bone Metastasis). Mol Cancer Ther., 13(12) December (2014); Verbovsek U. et al., Expression Analysis of All Protease Genes Reveals Cathepsin K to Be Overexpressed in Glioblastoma. PLoS ONE 9(10): e111819. doi:10.1371/journal.pone.0111819; Dai R. et al., Cathepsin K: The Action in and Beyond Bone. Front. Cell Dev. Biol. 8:433. doi: 10.3389/fcell.2020.00433). In a yet further embodiment, Cathepsin L is a cysteine protease having substrates that include SEQ ID NO: 192, SEQ ID NO: 193, SEQ ID NO: 194, and SEQ ID NO: 195 (see MEROPS ID: C01.032). Skilled persons will understand that Cathepsin L is elevated in various cancers including breast, lung, colon, pancreatic, and ovarian cancer. In a yet further embodiment, Cathepsin S is a cysteine protease having substrates that include SEQ ID NO: 196 and SEQ ID NO: 197 (see MEROPS ID: C01.34). Skilled persons will understand that Cathepsin S is elevated in a broad range of cancers including brain, liver, pancreatic, and gastric cancer.

In some embodiments, the protease substrate motif is configured as a substrate of kallikreins (KLKs). KLKs are known by skilled persons as biomarkers of cancer (Diamandis E. P. and Yousef G. M., Human Tissue Kallikreins: A Family of New Cancer Biomarkers, Clinical Chemistry 48:8; 1198-1205 (2002)). In some embodiments, prostate-specific antigen (PSA) (also known as kallikrein-3 (KLK3), gamma-seminoproteinn, and P-30 antigen) is a serine protease having substrates that include SEQ ID NO: 198, SEQ ID NO: 199, and SEQ ID NO: 200 (see MEROPS ID: S01.162). Skilled persons will understand that PSA is elevated in cases of prostate cancer and other prostate disorders (Catalona W. J. et al., Comparison of Digital Rectal Examination and Serum Prostate Specific Antigen in the Early Detection of Prostate Cancer: Results of a Multicenter Clinical Trial of 6,630 Men. Journal of Urology. 151; 5: 1283-1290 (1994)). In a further embodiment, kallikrein-2 (KLK2) (also known as human kallikrein 2 (hK2) and human glandular kallikrein-1 (hGK-1)) is a serine protease having substrates that include SEQ ID NO: 201 and SEQ ID NO: 202 (see MEROPS ID: S01.161). Skilled persons will understand that KLK2 is elevated in cases of prostate cancer (Borgono C. A. and Diamandis E. P., The Emerging Role of Human Tissue Kallikreins in Cancer. Nature Rev. Cancer, Vol. 4:876-890 November (2004)).

In some embodiments, the protease substrate motif is configured as a substrate of beta-secretase 1 (also known as beta-site APP cleaving enzyme 1 (BACE 1) and memapsin-2). Beta-secretase 1 is an aspartic acid protease having a substrate that includes SEQ ID NO: 203 (see MEROPS ID: A01.004). Skilled persons will understand that beta-secretase 1 is elevated in Alzheimer's disease (Repetto E. et al., BACE1 Overexpression Regulates Amyloid Precursor Protein Cleavage and Interaction with the ShcA Adapter. Ann. N.Y. Acad. Sci. 1030: 330-338 (2004)).

In some embodiments, the protease substrate motif is configured as a substrate of matriptase-1 (also known as suppressor of tumorigenicity 14 protein (ST14). Matriptase-1 is a serine protease having substrates that include SEQ ID NO: 204 and SEQ ID NO: 205 (see MEROPS ID: S01.302). Skilled persons will understand that matriptase-1 is overexpressed in cancers including breast, colon, ovarian, and prostate cancer (Uhland K., Matriptase and its putative role in cancer. Cell. Mol. Life Sci., 63:2968-2978 (2006)).

Skilled persons will understand that the self-assembling peptides disclosed herein may be readily produced by custom polypeptide synthesis, as described herein. Custom polypeptide synthesis allows for various combinations of β-strand motifs and hydrophilic motifs to be combined with any one of the substrate motifs disclosed herein and synthesized as a contiguous polypeptide. Thus, in some embodiments, a self-assembling polypeptide for detecting protease selected from any one of: a Furin protease, an ADAMs protease, a MMP protease, a LGMN, a Cathepsin protease, a KLK protease, a Beta-secretase 1 protease, and a matriptase protease may comprise any one of the embodiments disclosed in Table 1. As used in Table 1, “B” followed by a number indicates the sequence identifier (i.e., SEQ ID NO:) of a β-strand motif amino acid sequence. For example, “β5” refers to a β-strand motif comprising SEQ ID NO: 5. As used in Table 1, “H” followed a number indicates the sequence identifier of a hydrophilic motif amino acid sequence. For example, “H50” refers to a hydrophilic motif comprising SEQ ID NO: 50. As used in Table 1, “S” indicates any one of the protease substrate motifs disclosed herein. Thus, as used in Table 1, the combination “B5SH50” refers to a self-assembling polypeptide, from N-terminus to C-terminus, comprising SEQ ID NO: 5, any one of SEQ ID NOs: 145 to 205, and SEQ ID NO:50.

TABLE 1
Combinations of β-strand motif and hydrophilic
motif for detecting a protease having a substrate that
comprises a selected substrate motif S

| | | | | | |
|---|---|---|---|---|---|
| B1SH100 | B10SH56 | B3SH142 | B5SH102 | B6SH58 | B8SH144 |
| B1SH101 | B10SH57 | B3SH143 | B5SH103 | B6SH59 | B8SH15 |
| B1SH102 | B10SH58 | B3SH144 | B5SH104 | B6SH60 | B8SH16 |
| B1SH103 | B10SH59 | B3SH15 | B5SH105 | B6SH61 | B8SH17 |
| B1SH104 | B10SH60 | B3SH16 | B5SH106 | B6SH62 | B8SH18 |
| B1SH105 | B10SH61 | B3SH17 | B5SH107 | B6SH63 | B8SH19 |
| B1SH106 | B10SH62 | B3SH18 | B5SH108 | B6SH64 | B8SH20 |
| B1SH107 | B10SH63 | B3SH19 | B5SH109 | B6SH65 | B8SH21 |
| B1SH108 | B10SH64 | B3SH20 | B5SH11 | B6SH66 | B8SH22 |
| B1SH109 | B10SH65 | B3SH21 | B5SH110 | B6SH67 | B8SH23 |
| B1SH11 | B10SH66 | B3SH22 | B5SH111 | B6SH68 | B8SH24 |
| B1SH110 | B10SH67 | B3SH23 | B5SH112 | B6SH69 | B8SH25 |
| B1SH111 | B10SH68 | B3SH24 | B5SH113 | B6SH70 | B8SH26 |
| B1SH112 | B10SH69 | B3SH25 | B5SH114 | B6SH71 | B8SH27 |
| B1SH113 | B10SH70 | B3SH26 | B5SH115 | B6SH72 | B8SH28 |
| B1SH114 | B10SH71 | B3SH27 | B5SH116 | B6SH73 | B8SH29 |
| B1SH115 | B10SH72 | B3SH28 | B5SH117 | B6SH74 | B8SH30 |
| B1SH116 | B10SH73 | B3SH29 | B5SH118 | B6SH75 | B8SH31 |
| B1SH117 | B10SH74 | B3SH30 | B5SH119 | B6SH76 | B8SH32 |
| B1SH118 | B10SH75 | B3SH31 | B5SH12 | B6SH77 | B8SH33 |
| B1SH119 | B10SH76 | B3SH32 | B5SH120 | B6SH78 | B8SH34 |
| B1SH12 | B10SH77 | B3SH33 | B5SH121 | B6SH79 | B8SH35 |
| B1SH120 | B10SH78 | B3SH34 | B5SH122 | B6SH80 | B8SH36 |
| B1SH121 | B10SH79 | B3SH35 | B5SH123 | B6SH81 | B8SH37 |
| B1SH122 | B10SH80 | B3SH36 | B5SH124 | B6SH82 | B8SH38 |
| B1SH123 | B10SH81 | B3SH37 | B5SH125 | B6SH83 | B8SH39 |
| B1SH124 | B10SH82 | B3SH38 | B5SH126 | B6SH84 | B8SH40 |
| B1SH125 | B10SH83 | B3SH39 | B5SH127 | B6SH85 | B8SH41 |
| B1SH126 | B10SH84 | B3SH40 | B5SH128 | B6SH86 | B8SH42 |
| B1SH127 | B10SH85 | B3SH41 | B5SH129 | B6SH87 | B8SH43 |
| B1SH128 | B10SH86 | B3SH42 | B5SH13 | B6SH88 | B8SH44 |
| B1SH129 | B10SH87 | B3SH43 | B5SH130 | B6SH89 | B8SH45 |
| B1SH13 | B10SH88 | B3SH44 | B5SH131 | B6SH90 | B8SH46 |
| B1SH130 | B10SH89 | B3SH45 | B5SH132 | B6SH91 | B8SH47 |
| B1SH131 | B10SH90 | B3SH46 | B5SH133 | B6SH92 | B8SH48 |
| B1SH132 | B10SH91 | B3SH47 | B5SH134 | B6SH93 | B8SH49 |
| B1SH133 | B10SH92 | B3SH48 | B5SH135 | B6SH94 | B8SH50 |
| B1SH134 | B10SH93 | B3SH49 | B5SH136 | B6SH95 | B8SH51 |
| B1SH135 | B10SH94 | B3SH50 | B5SH137 | B6SH96 | B8SH52 |
| B1SH136 | B10SH95 | B3SH51 | B5SH138 | B6SH97 | B8SH53 |
| B1SH137 | B10SH96 | B3SH52 | B5SH139 | B6SH98 | B8SH54 |
| B1SH138 | B10SH97 | B3SH53 | B5SH14 | B6SH99 | B8SH55 |
| B1SH139 | B10SH98 | B3SH54 | B5SH140 | B7SH100 | B8SH56 |
| B1SH14 | B10SH99 | B3SH55 | B5SH141 | B7SH101 | B8SH57 |
| B1SH140 | B2SH100 | B3SH56 | B5SH142 | B7SH102 | B8SH58 |
| B1SH141 | B2SH101 | B3SH57 | B5SH143 | B7SH103 | B8SH59 |
| B1SH142 | B2SH102 | B3SH58 | B5SH144 | B7SH104 | B8SH60 |
| B1SH143 | B2SH103 | B3SH59 | B5SH15 | B7SH105 | B8SH61 |
| B1SH144 | B2SH104 | B3SH60 | B5SH16 | B7SH106 | B8SH62 |
| B1SH15 | B2SH105 | B3SH61 | B5SH17 | B7SH107 | B8SH63 |
| B1SH16 | B2SH106 | B3SH62 | B5SH18 | B7SH108 | B8SH64 |
| B1SH17 | B2SH107 | B3SH63 | B5SH19 | B7SH109 | B8SH65 |
| B1SH18 | B2SH108 | B3SH64 | B5SH20 | B7SH11 | B8SH66 |
| B1SH19 | B2SH109 | B3SH65 | B5SH21 | B7SH110 | B8SH67 |
| B1SH20 | B2SH11 | B3SH66 | B5SH22 | B7SH111 | B8SH68 |
| B1SH21 | B2SH110 | B3SH67 | B5SH23 | B7SH112 | B8SH69 |
| B1SH22 | B2SH111 | B3SH68 | B5SH24 | B7SH113 | B8SH70 |
| B1SH23 | B2SH112 | B3SH69 | B5SH25 | B7SH114 | B8SH71 |
| B1SH24 | B2SH113 | B3SH70 | B5SH26 | B7SH115 | B8SH72 |
| B1SH25 | B2SH114 | B3SH71 | B5SH27 | B7SH116 | B8SH73 |
| B1SH26 | B2SH115 | B3SH72 | B5SH28 | B7SH117 | B8SH74 |
| B1SH27 | B2SH116 | B3SH73 | B5SH29 | B7SH118 | B8SH75 |
| B1SH28 | B2SH117 | B3SH74 | B5SH30 | B7SH119 | B8SH76 |
| B1SH29 | B2SH118 | B3SH75 | B5SH31 | B7SH12 | B8SH77 |
| B1SH30 | B2SH119 | B3SH76 | B5SH32 | B7SH120 | B8SH78 |
| B1SH31 | B2SH12 | B3SH77 | B5SH33 | B7SH121 | B8SH79 |
| B1SH32 | B2SH120 | B3SH78 | B5SH34 | B7SH122 | B8SH80 |
| B1SH33 | B2SH121 | B3SH79 | B5SH35 | B7SH123 | B8SH81 |
| B1SH34 | B2SH122 | B3SH80 | B5SH36 | B7SH124 | B8SH82 |
| B1SH35 | B2SH123 | B3SH81 | B5SH37 | B7SH125 | B8SH83 |
| B1SH36 | B2SH124 | B3SH82 | B5SH38 | B7SH126 | B8SH84 |
| B1SH37 | B2SH125 | B3SH83 | B5SH39 | B7SH127 | B8SH85 |
| B1SH38 | B2SH126 | B3SH84 | B5SH40 | B7SH128 | B8SH86 |
| B1SH39 | B2SH127 | B3SH85 | B5SH41 | B7SH129 | B8SH87 |
| B1SH40 | B2SH128 | B3SH86 | B5SH42 | B7SH13 | B8SH88 |
| B1SH41 | B2SH129 | B3SH87 | B5SH43 | B7SH130 | B8SH89 |
| B1SH42 | B2SH13 | B3SH88 | B5SH44 | B7SH131 | B8SH90 |
| B1SH43 | B2SH130 | B3SH89 | B5SH45 | B7SH132 | B8SH91 |
| B1SH44 | B2SH131 | B3SH90 | B5SH46 | B7SH133 | B8SH92 |
| B1SH45 | B2SH132 | B3SH91 | B5SH47 | B7SH134 | B8SH93 |
| B1SH46 | B2SH133 | B3SH92 | B5SH48 | B7SH135 | B8SH94 |
| B1SH47 | B2SH134 | B3SH93 | B5SH49 | B7SH136 | B8SH95 |
| B1SH48 | B2SH135 | B3SH94 | B5SH50 | B7SH137 | B8SH96 |
| B1SH49 | B2SH136 | B3SH95 | B5SH51 | B7SH138 | B8SH97 |
| B1SH50 | B2SH137 | B3SH96 | B5SH52 | B7SH139 | B8SH98 |
| B1SH51 | B2SH138 | B3SH97 | B5SH53 | B7SH14 | B8SH99 |
| B1SH52 | B2SH139 | B3SH98 | B5SH54 | B7SH140 | B9SH100 |
| B1SH53 | B2SH14 | B3SH99 | B5SH55 | B7SH141 | B9SH101 |
| B1SH54 | B2SH140 | B4SH100 | B5SH56 | B7SH142 | B9SH102 |
| B1SH55 | B2SH141 | B4SH101 | B5SH57 | B7SH143 | B9SH103 |
| B1SH56 | B2SH142 | B4SH102 | B5SH58 | B7SH144 | B9SH104 |
| B1SH57 | B2SH143 | B4SH103 | B5SH59 | B7SH15 | B9SH105 |
| B1SH58 | B2SH144 | B4SH104 | B5SH60 | B7SH16 | B9SH106 |
| B1SH59 | B2SH15 | B4SH105 | B5SH61 | B7SH17 | B9SH107 |
| B1SH60 | B2SH16 | B4SH106 | B5SH62 | B7SH18 | B9SH108 |
| B1SH61 | B2SH17 | B4SH107 | B5SH63 | B7SH19 | B9SH109 |
| B1SH62 | B2SH18 | B4SH108 | B5SH64 | B7SH20 | B9SH11 |
| B1SH63 | B2SH19 | B4SH109 | B5SH65 | B7SH21 | B9SH110 |
| B1SH64 | B2SH20 | B4SH11 | B5SH66 | B7SH22 | B9SH111 |
| B1SH65 | B2SH21 | B4SH110 | B5SH67 | B7SH23 | B9SH112 |
| B1SH66 | B2SH22 | B4SH111 | B5SH68 | B7SH24 | B9SH113 |
| B1SH67 | B2SH23 | B4SH112 | B5SH69 | B7SH25 | B9SH114 |
| B1SH68 | B2SH24 | B4SH113 | B5SH70 | B7SH26 | B9SH115 |
| B1SH69 | B2SH25 | B4SH114 | B5SH71 | B7SH27 | B9SH116 |
| B1SH70 | B2SH26 | B4SH115 | B5SH72 | B7SH28 | B9SH117 |
| B1SH71 | B2SH27 | B4SH116 | B5SH73 | B7SH29 | B9SH118 |
| B1SH72 | B2SH28 | B4SH117 | B5SH74 | B7SH30 | B9SH119 |
| B1SH73 | B2SH29 | B4SH118 | B5SH75 | B7SH31 | B9SH12 |
| B1SH74 | B2SH30 | B4SH119 | B5SH76 | B7SH32 | B9SH120 |
| B1SH75 | B2SH31 | B4SH12 | B5SH77 | B7SH33 | B9SH121 |
| B1SH76 | B2SH32 | B4SH120 | B5SH78 | B7SH34 | B9SH122 |
| B1SH77 | B2SH33 | B4SH121 | B5SH79 | B7SH35 | B9SH123 |
| B1SH78 | B2SH34 | B4SH122 | B5SH80 | B7SH36 | B9SH124 |
| B1SH79 | B2SH35 | B4SH123 | B5SH81 | B7SH37 | B9SH125 |
| B1SH80 | B2SH36 | B4SH124 | B5SH82 | B7SH38 | B9SH126 |
| B1SH81 | B2SH37 | B4SH125 | B5SH83 | B7SH39 | B9SH127 |
| B1SH82 | B2SH38 | B4SH126 | B5SH84 | B7SH40 | B9SH128 |
| B1SH83 | B2SH39 | B4SH127 | B5SH85 | B7SH41 | B9SH129 |
| B1SH84 | B2SH40 | B4SH128 | B5SH86 | B7SH42 | B9SH13 |
| B1SH85 | B2SH41 | B4SH129 | B5SH87 | B7SH43 | B9SH130 |
| B1SH86 | B2SH42 | B4SH13 | B5SH88 | B7SH44 | B9SH131 |
| B1SH87 | B2SH43 | B4SH130 | B5SH89 | B7SH45 | B9SH132 |
| B1SH88 | B2SH44 | B4SH131 | B5SH90 | B7SH46 | B9SH133 |
| B1SH89 | B2SH45 | B4SH132 | B5SH91 | B7SH47 | B9SH134 |
| B1SH90 | B2SH46 | B4SH133 | B5SH92 | B7SH48 | B9SH135 |
| B1SH91 | B2SH47 | B4SH134 | B5SH93 | B7SH49 | B9SH136 |
| B1SH92 | B2SH48 | B4SH135 | B5SH94 | B7SH50 | B9SH137 |
| B1SH93 | B2SH49 | B4SH136 | B5SH95 | B7SH51 | B9SH138 |
| B1SH94 | B2SH50 | B4SH137 | B5SH96 | B7SH52 | B9SH139 |
| B1SH95 | B2SH51 | B4SH138 | B5SH97 | B7SH53 | B9SH14 |
| B1SH96 | B2SH52 | B4SH139 | B5SH98 | B7SH54 | B9SH140 |
| B1SH97 | B2SH53 | B4SH14 | B5SH99 | B7SH55 | B9SH141 |
| B1SH98 | B2SH54 | B4SH140 | B6SH100 | B7SH56 | B9SH142 |
| B1SH99 | B2SH55 | B4SH141 | B6SH101 | B7SH57 | B9SH143 |
| B10SH100 | B2SH56 | B4SH142 | B6SH102 | B7SH58 | B9SH144 |
| B10SH101 | B2SH57 | B4SH143 | B6SH103 | B7SH59 | B9SH15 |
| B10SH102 | B2SH58 | B4SH144 | B6SH104 | B7SH60 | B9SH16 |
| B10SH103 | B2SH59 | B4SH15 | B6SH105 | B7SH61 | B9SH17 |
| B10SH104 | B2SH60 | B4SH16 | B6SH106 | B7SH62 | B9SH18 |
| B10SH105 | B2SH61 | B4SH17 | B6SH107 | B7SH63 | B9SH19 |
| B10SH106 | B2SH62 | B4SH18 | B6SH108 | B7SH64 | B9SH20 |
| B10SH107 | B2SH63 | B4SH19 | B6SH109 | B7SH65 | B9SH21 |
| B10SH108 | B2SH64 | B4SH20 | B6SH11 | B7SH66 | B9SH22 |
| B10SH109 | B2SH65 | B4SH21 | B6SH110 | B7SH67 | B9SH23 |
| B10SH11 | B2SH66 | B4SH22 | B6SH111 | B7SH68 | B9SH24 |
| B10SH110 | B2SH67 | B4SH23 | B6SH112 | B7SH69 | B9SH25 |
| B10SH111 | B2SH68 | B4SH24 | B6SH113 | B7SH70 | B9SH26 |
| B10SH112 | B2SH69 | B4SH25 | B6SH114 | B7SH71 | B9SH27 |
| B10SH113 | B2SH70 | B4SH26 | B6SH115 | B7SH72 | B9SH28 |
| B10SH114 | B2SH71 | B4SH27 | B6SH116 | B7SH73 | B9SH29 |
| B10SH115 | B2SH72 | B4SH28 | B6SH117 | B7SH74 | B9SH30 |
| B10SH116 | B2SH73 | B4SH29 | B6SH118 | B7SH75 | B9SH31 |
| B10SH117 | B2SH74 | B4SH30 | B6SH119 | B7SH76 | B9SH32 |
| B10SH118 | B2SH75 | B4SH31 | B6SH12 | B7SH77 | B9SH33 |
| B10SH119 | B2SH76 | B4SH32 | B6SH120 | B7SH78 | B9SH34 |
| B10SH12 | B2SH77 | B4SH33 | B6SH121 | B7SH79 | B9SH35 |
| B10SH120 | B2SH78 | B4SH34 | B6SH122 | B7SH80 | B9SH36 |
| B10SH121 | B2SH79 | B4SH35 | B6SH123 | B7SH81 | B9SH37 |
| B10SH122 | B2SH80 | B4SH36 | B6SH124 | B7SH82 | B9SH38 |
| B10SH123 | B2SH81 | B4SH37 | B6SH125 | B7SH83 | B9SH39 |
| B10SH124 | B2SH82 | B4SH38 | B6SH126 | B7SH84 | B9SH40 |
| B10SH125 | B2SH83 | B4SH39 | B6SH127 | B7SH85 | B9SH41 |
| B10SH126 | B2SH84 | B4SH40 | B6SH128 | B7SH86 | B9SH42 |
| B10SH127 | B2SH85 | B4SH41 | B6SH129 | B7SH87 | B9SH43 |
| B10SH128 | B2SH86 | B4SH42 | B6SH13 | B7SH88 | B9SH44 |
| B10SH129 | B2SH87 | B4SH43 | B6SH130 | B7SH89 | B9SH45 |
| B10SH13 | B2SH88 | B4SH44 | B6SH131 | B7SH90 | B9SH46 |
| B10SH130 | B2SH89 | B4SH45 | B6SH132 | B7SH91 | B9SH47 |
| B10SH131 | B2SH90 | B4SH46 | B6SH133 | B7SH92 | B9SH48 |
| B10SH132 | B2SH91 | B4SH47 | B6SH134 | B7SH93 | B9SH49 |
| B10SH133 | B2SH92 | B4SH48 | B6SH135 | B7SH94 | B9SH50 |
| B10SH134 | B2SH93 | B4SH49 | B6SH136 | B7SH95 | B9SH51 |
| B10SH135 | B2SH94 | B4SH50 | B6SH137 | B7SH96 | B9SH52 |
| B10SH136 | B2SH95 | B4SH51 | B6SH138 | B7SH97 | B9SH53 |
| B10SH137 | B2SH96 | B4SH52 | B6SH139 | B7SH98 | B9SH54 |
| B10SH138 | B2SH97 | B4SH53 | B6SH14 | B7SH99 | B9SH55 |
| B10SH139 | B2SH98 | B4SH54 | B6SH140 | B8SH100 | B9SH56 |
| B10SH14 | B2SH99 | B4SH55 | B6SH141 | B8SH101 | B9SH57 |
| B10SH140 | B3SH100 | B4SH56 | B6SH142 | B8SH102 | B9SH58 |
| B10SH141 | B3SH101 | B4SH57 | B6SH143 | B8SH103 | B9SH59 |
| B10SH142 | B3SH102 | B4SH58 | B6SH144 | B8SH104 | B9SH60 |
| B10SH143 | B3SH103 | B4SH59 | B6SH15 | B8SH105 | B9SH61 |
| B10SH144 | B3SH104 | B4SH60 | B6SH16 | B8SH106 | B9SH62 |
| B10SH15 | B3SH105 | B4SH61 | B6SH17 | B8SH107 | B9SH63 |
| B10SH16 | B3SH106 | B4SH62 | B6SH18 | B8SH108 | B9SH64 |
| B10SH17 | B3SH107 | B4SH63 | B6SH19 | B8SH109 | B9SH65 |
| B10SH18 | B3SH108 | B4SH64 | B6SH20 | B8SH11 | B9SH66 |
| B10SH19 | B3SH109 | B4SH65 | B6SH21 | B8SH110 | B9SH67 |
| B10SH20 | B3SH11 | B4SH66 | B6SH22 | B8SH111 | B9SH68 |
| B10SH21 | B3SH110 | B4SH67 | B6SH23 | B8SH112 | B9SH69 |
| B10SH22 | B3SH111 | B4SH68 | B6SH24 | B8SH113 | B9SH70 |
| B10SH23 | B3SH112 | B4SH69 | B6SH25 | B8SH114 | B9SH71 |
| B10SH24 | B3SH113 | B4SH70 | B6SH26 | B8SH115 | B9SH72 |
| B10SH25 | B3SH114 | B4SH71 | B6SH27 | B8SH116 | B9SH73 |
| B10SH26 | B3SH115 | B4SH72 | B6SH28 | B8SH117 | B9SH74 |
| B10SH27 | B3SH116 | B4SH73 | B6SH29 | B8SH118 | B9SH75 |
| B10SH28 | B3SH117 | B4SH74 | B6SH30 | B8SH119 | B9SH76 |
| B10SH29 | B3SH118 | B4SH75 | B6SH31 | B8SH12 | B9SH77 |
| B10SH30 | B3SH119 | B4SH76 | B6SH32 | B8SH120 | B9SH78 |
| B10SH31 | B3SH12 | B4SH77 | B6SH33 | B8SH121 | B9SH79 |
| B10SH32 | B3SH120 | B4SH78 | B6SH34 | B8SH122 | B9SH80 |
| B10SH33 | B3SH121 | B4SH79 | B6SH35 | B8SH123 | B9SH81 |
| B10SH34 | B3SH122 | B4SH80 | B6SH36 | B8SH124 | B9SH82 |
| B10SH35 | B3SH123 | B4SH81 | B6SH37 | B8SH125 | B9SH83 |
| B10SH36 | B3SH124 | B4SH82 | B6SH38 | B8SH126 | B9SH84 |
| B10SH37 | B3SH125 | B4SH83 | B6SH39 | B8SH127 | B9SH85 |
| B10SH38 | B3SH126 | B4SH84 | B6SH40 | B8SH128 | B9SH86 |
| B10SH39 | B3SH127 | B4SH85 | B6SH41 | B8SH129 | B9SH87 |
| B10SH40 | B3SH128 | B4SH86 | B6SH42 | B8SH13 | B9SH88 |
| B10SH41 | B3SH129 | B4SH87 | B6SH43 | B8SH130 | B9SH89 |
| B10SH42 | B3SH13 | B4SH88 | B6SH44 | B8SH131 | B9SH90 |
| B10SH43 | B3SH130 | B4SH89 | B6SH45 | B8SH132 | B9SH91 |
| B10SH44 | B3SH131 | B4SH90 | B6SH46 | B8SH133 | B9SH92 |
| B10SH45 | B3SH132 | B4SH91 | B6SH47 | B8SH134 | B9SH93 |
| B10SH46 | B3SH133 | B4SH92 | B6SH48 | B8SH135 | B9SH94 |
| B10SH47 | B3SH134 | B4SH93 | B6SH49 | B8SH136 | B9SH95 |
| B10SH48 | B3SH135 | B4SH94 | B6SH50 | B8SH137 | B9SH96 |
| B10SH49 | B3SH136 | B4SH95 | B6SH51 | B8SH138 | B9SH97 |
| B10SH50 | B3SH137 | B4SH96 | B6SH52 | B8SH139 | B9SH98 |
| B10SH51 | B3SH138 | B4SH97 | B6SH53 | B8SH14 | B9SH99 |
| B10SH52 | B3SH139 | B4SH98 | B6SH54 | B8SH140 | |
| B10SH53 | B3SH14 | B4SH99 | B6SH55 | B8SH141 | |
| B10SH54 | B3SH140 | B5SH100 | B6SH56 | B8SH142 | |
| B10SH55 | B3SH141 | B5SH101 | B6SH57 | B8SH143 | |

In an exemplary embodiment, the self-assembling polypeptide of any of the embodiments disclosed herein may be utilized as means for detecting a protease in an aqueous milieu by the protease triggering the enzyme-instructed self-assembly of the self-assembling polypeptide to form an anti-parallel β-sheet structure. In the exemplary embodiment, the aqueous milieu comprises a β-sheet intercalating dye that emits fluorescent light upon intercalating with the anti-parallel β-sheet structure. In some embodiments, detecting the protease is utilized as means for detecting a cancer selected from any of: a liver cancer, a skin cancer, a cervical cancer, a brain cancer, a lung cancer, a stomach cancer, a bile-duct cancer, a gastric cancer, a pancreatic cancer, a bladder cancer, a breast cancer, a lung cancer, a prostate cancer, a kidney cancer, a brain cancer, a colorectal cancer, an ovarian cancer, a thyroid cancer, a melanoma cancer, and a thyroid cancer. In some embodiments, detecting the protease is utilized as means for detecting a disease, disorder, or syndrome selected from any of: a pre-cancerous breast hyperplasia, a prostate disorder, an Alzheimer's disease, an acute coronary disease, an atherosclerosis disease, an arthritis disease, an osteoarthritis disease, an osteoporosis disease, and an acute coronary syndrome.

In an exemplary embodiment, a method for detecting proteolytic cleavage by enzyme-instructed β-sheet formation comprises administering, into an aqueous milieu, a set of one or more self-assembling polypeptides of any of the embodiments disclosed herein. A β-sheet intercalating dye is administered into the aqueous milieu, the β-sheet intercalating dye being configured to emit a fluorescent signal upon forming a complex with one or more anti-parallel β-sheet structures formed by the self-assembly of β-strand motifs dissociated from their respective self-assembling polypeptides by proteolytic cleavage. A fluorescent signal is detected to indicate the presence of the protease in the aqueous milieu. In some embodiments, the β-sheet intercalating dye is selected from from a MCAAD-3 dye, a ThT dye, a Thioflavin T dye, a Thioflavin S dye, and a Congo Red dye. In some embodiments, the method is used to detect a cancer selected from any of: a liver cancer, a skin cancer, a cervical cancer, a brain cancer, a lung cancer, a stomach cancer, a bile-duct cancer, a gastric cancer, a pancreatic cancer, a bladder cancer, a breast cancer, a lung cancer, a prostate cancer, a kidney cancer, a brain cancer, a colorectal cancer, an ovarian cancer, a thyroid cancer, a melanoma cancer, and a thyroid cancer. In some embodiments, the method is used to detect a disease, disorder, or syndrome selected from any of: a pre-cancerous breast hyperplasia, a prostate disorder, an Alzheimer's disease, an acute coronary disease, an atherosclerosis disease, an arthritis disease, an osteoarthritis disease, an osteoporosis disease, and an acute coronary syndrome. In some embodiments, the an aqueous milieu is a plasma sample obtained from a subject.

In an exemplary embodiment, a kit, comprises a set of one or more self-assembling polypeptide of any of the embodiments disclosed herein and a β-sheet intercalating dye. In some of the embodiments, the β-sheet intercalating dye is selected from a MCAAD-3 dye, a ThT dye, a Thioflavin T dye, a Thioflavin S dye, and a Congo Red dye. In some embodiments, the kit is used to detect a cancer selected from any of: a liver cancer, a skin cancer, a cervical cancer, a brain cancer, a lung cancer, a stomach cancer, a bile-duct cancer, a gastric cancer, a pancreatic cancer, a bladder cancer, a breast cancer, a lung cancer, a prostate cancer, a kidney cancer, a brain cancer, a colorectal cancer, an ovarian cancer, a thyroid cancer, a melanoma cancer, and a thyroid cancer. In some embodiments, the kit is used to detect a disease, disorder, or syndrome selected from any of: a pre-cancerous breast hyperplasia, a prostate disorder, an Alzheimer's disease, an acute coronary disease, an atherosclerosis disease, an arthritis disease, an osteoarthritis disease, an osteoporosis disease, and an acute coronary syndrome.

A computer readable text file, entitled “Tech_2895_SEQ_LISTING_ST25.txt” created on or about Jul. 20, 2021, with a file size of 1 KB, contains the sequence listings for this application and is hereby incorporated by reference in its entirety.

The disclosed materials and methods relate to detecting protease activity. Some of the disclosed embodiments use cleavable, self-assembling probes that, upon being cleaved by a protease, self-assemble into anti-parallel beta-sheet structure capable of intercalating with fluorescent dye, allowing for detection protease activity.

Skilled persons will understand that the notation “/”, when set between standard single-letter code notation for amino acids incorporated into a peptide sequence, is an accepted convention marking a generally conserved protease cleavage site within the peptide sequence. In some embodiments, the substrate portion comprises a cysteine protease cleavage site. In some embodiments, the substrate portion comprises a legumain cleavage site. Skilled persons will understand that modifications to the peptide sequence of the substrate portion will facilitate detection of the cleavage activity of both characterized and uncharacterized proteases.

In some embodiments, an operatively connected ß-strand motif and substrate motif may be immobilized on solid supports (or “solid phase”) in lieu of a hydrophilic motif. Skilled persons will understand that examples of solid supports include microbeads, nanoparticles, dendrimers, surfaces, and membranes.

The technology described herein utilizes a distinct EISA method, namely enzyme-instructed β-sheet formation, for label-free fluorescent detection of protease activity. As disclosed herein, the method comprises utilizing commercially obtainable β-sheet forming peptides to provide self-assembly motifs without any special modification.

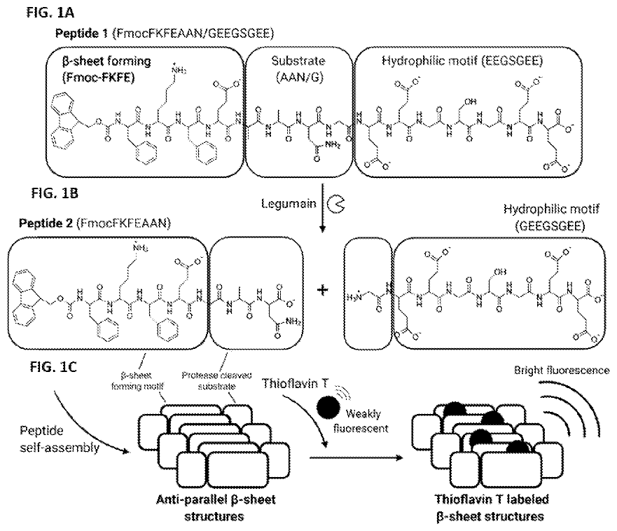

FIGS. 1A, 1B, and 1C are schematic representations of label-free protease detection using enzyme-instructed β-sheet formation. Molecular structures of peptide 1 (FIG. 1A) and peptide 2 (SEQ ID NO: 207) (FIG. 1B) formed upon hydrolysis of peptide 1 by legumain. FIG. 1C: Schematic showing the self-assembly of peptide 2 and Thioflavin T labeling of the β-sheet structures.

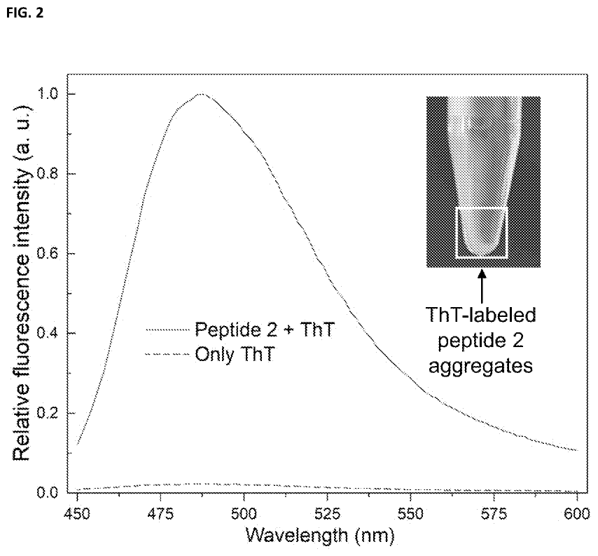

FIG. 2 is a graph of fluorescence spectra of ThT in the presence or absence of peptide 2 in assay buffer. Fluorescence spectra of ThT in the presence or absence of peptide 2 in assay buffer. Inset shows the ThT labeled peptide 2 aggregates collected by centrifugation.

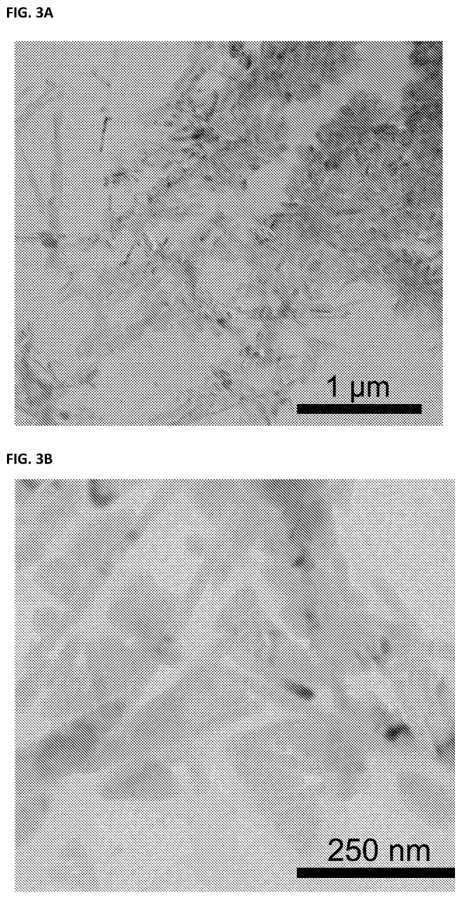

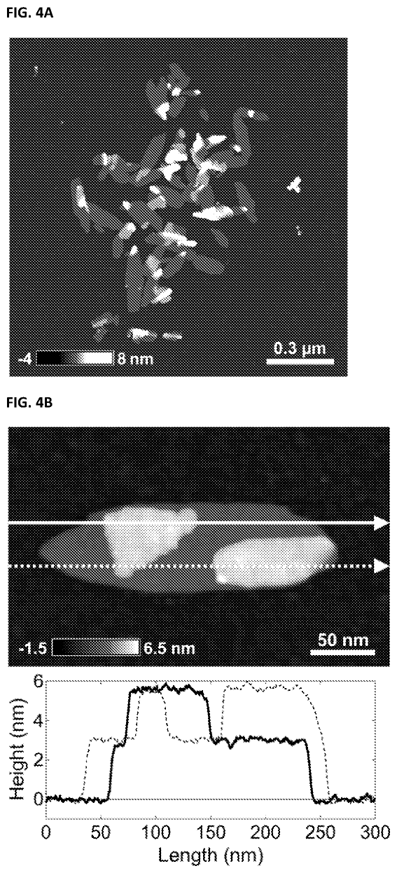

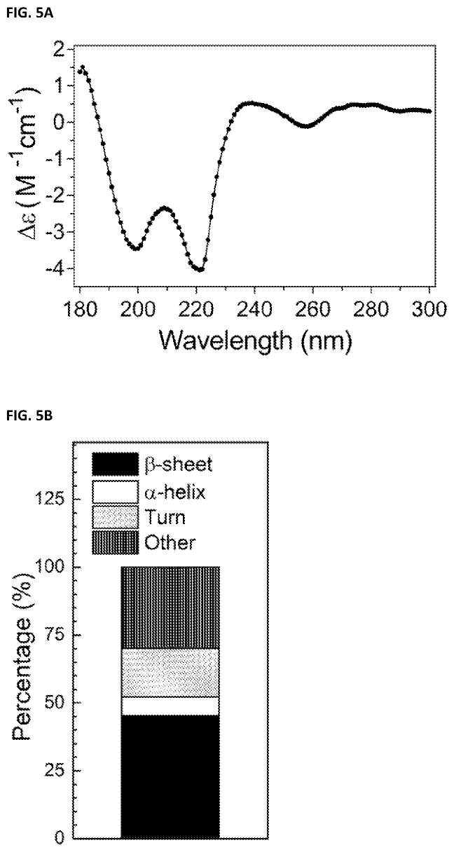

FIG. 3 shows TEM images of self-assembled structures of peptide 2; FIG. 4 shows AFM images of self-assembled structures of peptide 2; and FIG. 5 are line and percent graphs showing CD spectrum of peptide 2 suspended in assay buffer. TEM images (FIG. 3) and AFM images (FIG. 4) of self-assembled structures of peptide 2. FIG. 4B shows a high-resolution image of a nanoscale plate-like structure and two individual thickness profile measurements (the solid and dashed lines on the AFM image correspond to the solid and dashed lines of the Height versus Length line plot). FIG. 5A shows a CD spectrum of peptide 2 suspended in the assay buffer and FIG. 5B shows the secondary structure analysis of peptide 2 suspended in assay buffer based on CD results.

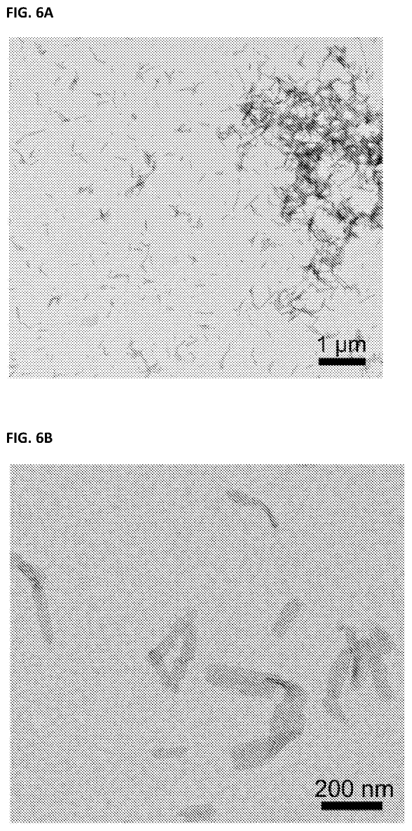

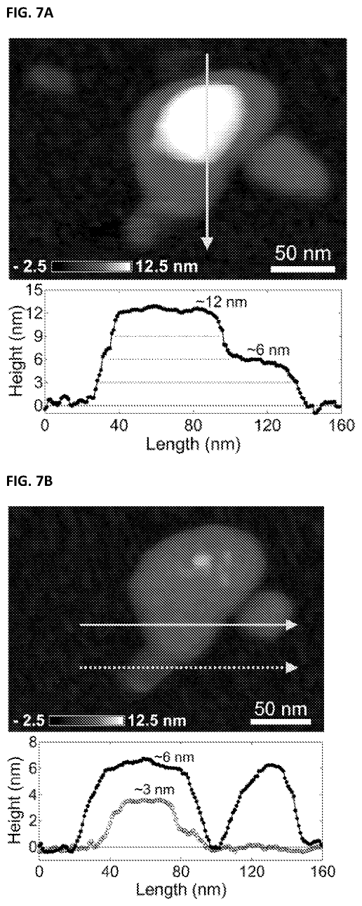

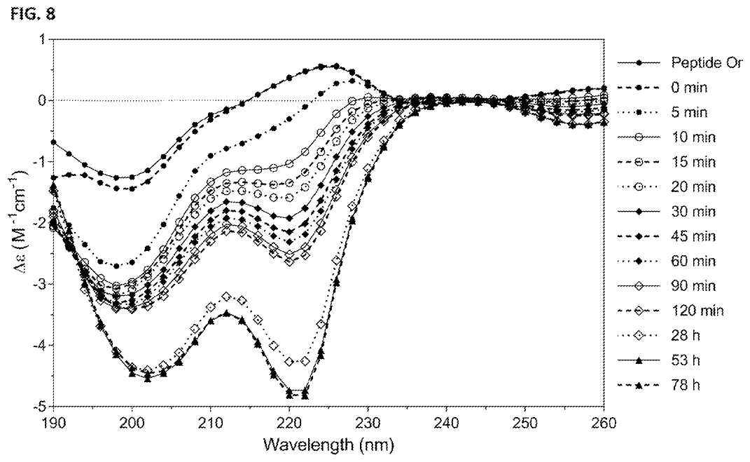

FIGS. 6A and 6B shows TEM images of peptide 1 incubated with legumain after bath sonication; FIGS. 7A and 7B shows AFM characterization of peptide 1 incubated with legumain; and FIG. 8 shows CD spectra of peptide 1 before and after legumain addition. FIG. 6: TEM images of peptide 1 incubated with 1000 ng/mL legumain at 37° C. for 2 hours after bath sonication. The low-resolution image in FIG. 6A shows a large aggregate formed by smaller plates and small platelets generated during the sonication process. The high-resolution image in FIG. 6B reveals the nano-platelet structure.

FIG. 7 shows AFM characterization of peptide 1 incubated with 1000 ng/mL legumain at 37° C. for 2 hours. The AFM images in FIGS. 7A and 7B were sequentially acquired and show the excavation of the layered peptide material of a nanoplatelet by the AFM probe. Height measurements corresponding to the measurement arrows on the AFM images show that the observed structures are composed of layers that are approximately 3 nm in thickness (the solid and dashed lines on the AFM images correspond to the solid (closed circle markers) and dashed (open circle markers) lines of the Height versus Length line plots). A schematic representation of the division of the layers is shown by the horizontal lines beneath the trace in FIG. 7A.

FIG. 8 shows the CD spectra of peptide 1 before and after legumain addition over 78 hours.

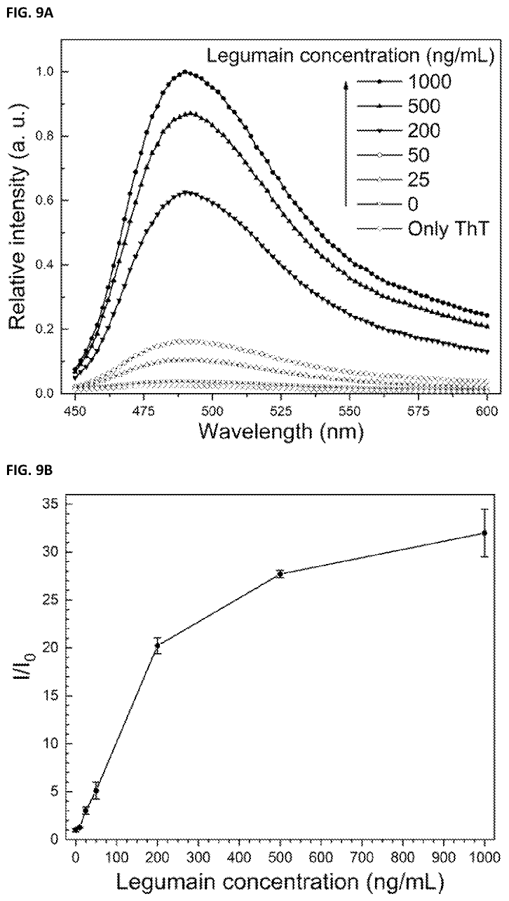

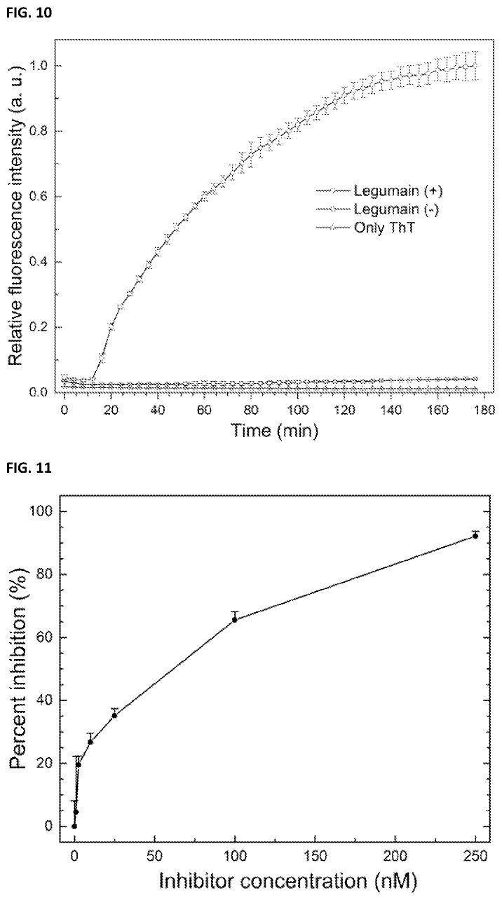

FIGS. 9A and 9B are line graphs showing, respectively, fluorescent intensity enhancement of ThT at different concentrations and absorbance spectra of ThT in the present or absence of peptide 1; and FIGS. 10 and 11 are line graphs showing, respectively, the kinetics of fluorescence signal change with or without legumain and the percent inhibition of the legumain activity at different inhibitor concentrations. Label-free legumain detection using peptide 1. FIG. 9A: Representative fluorescence spectra of ThT (90 μM) in the presence or absence of peptide 1 (1 mg/mL) and after 2 hours incubation with different amounts of legumain. FIG. 9B: Fluorescence intensity enhancement of ThT (I/I0) at different legumain concentrations. FIG. 10: Kinetics of fluorescence signal change with or without legumain (1000 ng/mL). FIG. 11: Percent inhibition of the legumain (1000 ng/mL) activity at different inhibitor (RR-11a) concentrations. Studies were run at least as triplicates. Error bars=1 standard deviation.

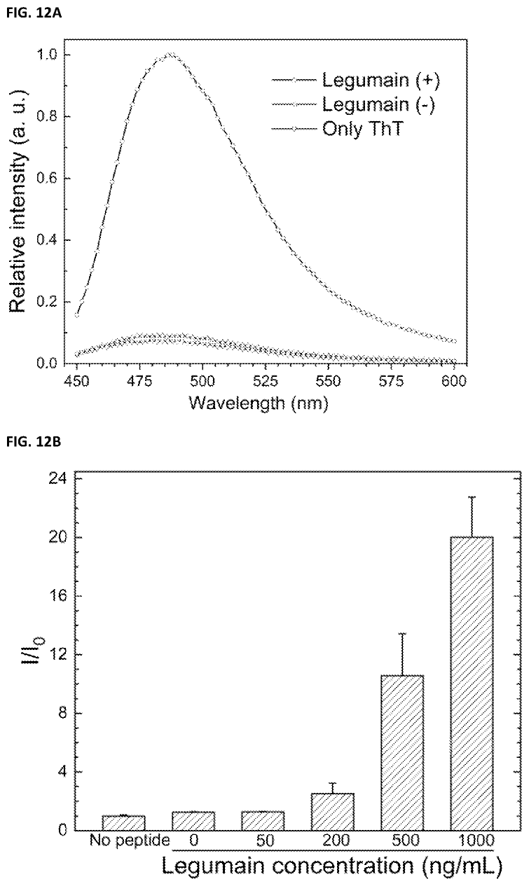

FIG. 12A is a line graph showing the representative fluorescence spectra of ThT in the presence of different peptide 1 amounts; and FIG. 12B is a line graph showing the relative fluorescence intensity of ThT in the presence of different amounts of peptide 1. Assay performance in human plasma. FIG. 12A: Representative fluorescence spectra of ThT (25 μM) in 10% plasma in the presence or absence of peptide 1 (1 mg/mL) and after 2 hours incubation with or without legumain (1000 ng/mL). FIG. 12B: Fluorescence in-tensity enhancement of ThT (I/I0) in 10% plasma at different legumain concentrations. Studies were run at least as triplicates. Error bars=1 standard deviation.

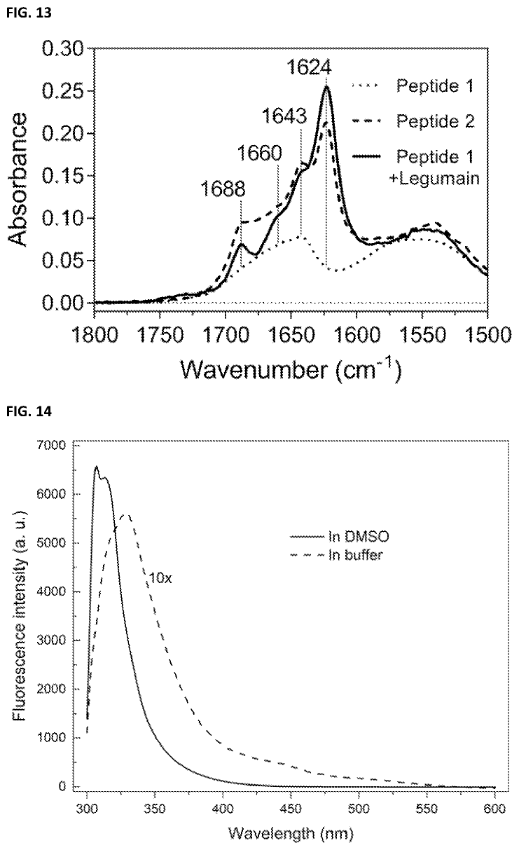

FIG. 13 is a FTIR spectra of peptide 1, before and after incubation with legumain and peptide 2; and FIG. 14 is a fluorescence spectra of peptide 2 in DMSO or buffer.

FIG. 14 is a line graph of representative fluorescence spectra of MCAAD-3 in the presence or absence of peptide 1 at about 1 mg/mL and after two hour incubation with different amounts of legumain.

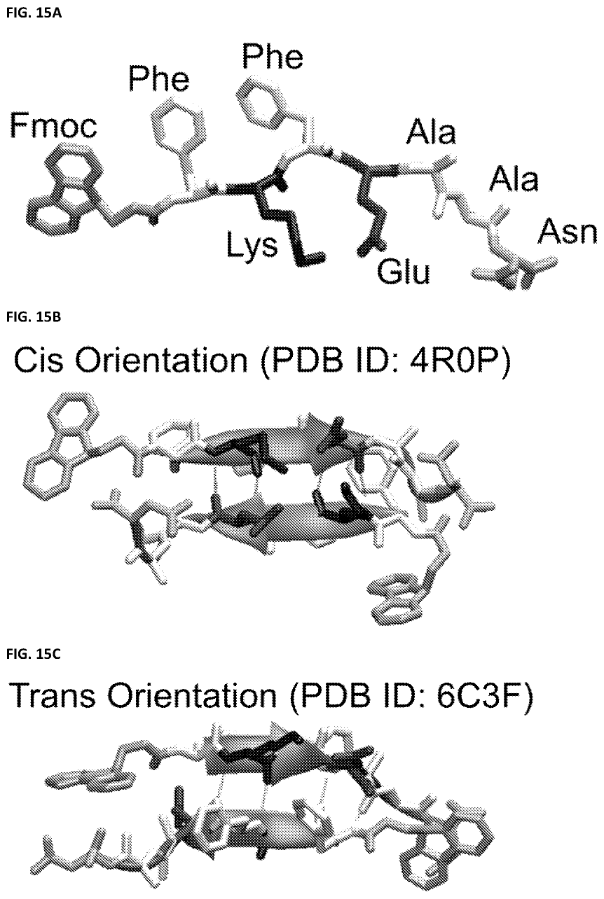

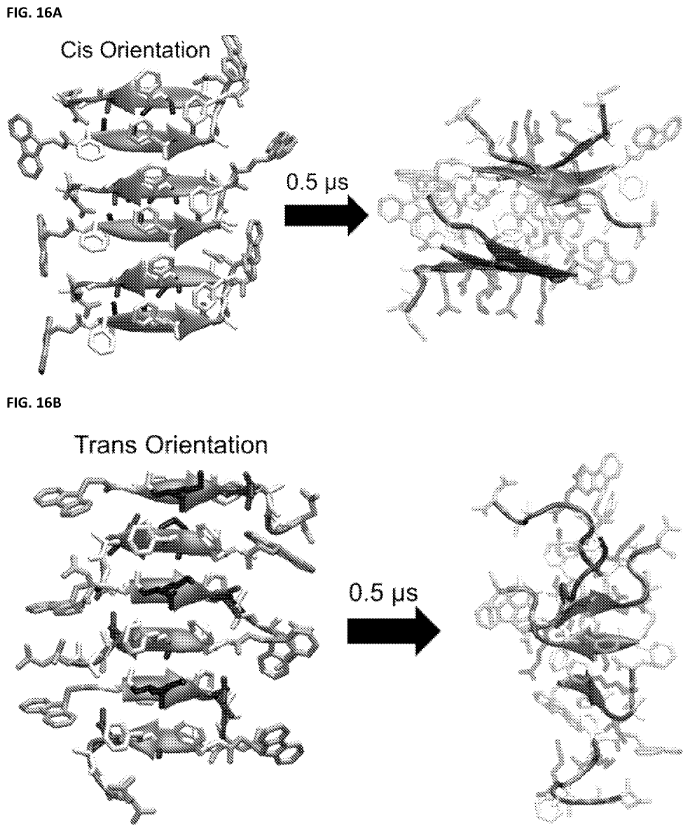

FIGS. 15 and 16 shows various stick models of peptide 2.

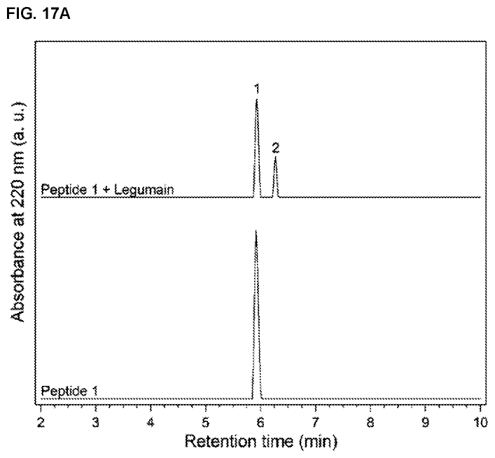

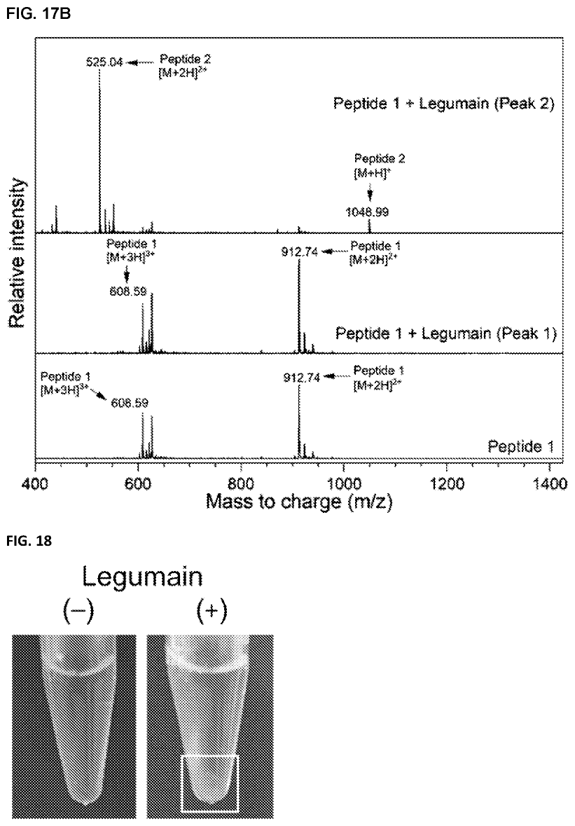

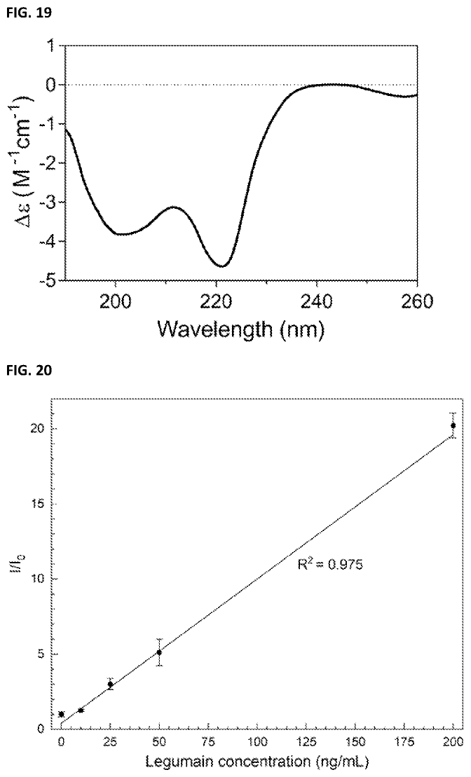

FIGS. 17A and 17B are, respectively, liquid chromatography (LC) and mass spectrometry (MS) data of peptide 1 before and after incubating with legumain.

FIG. 18 shows parallel photographs of peptide 1 solutions incubated with or without legumain after centrifugation showing ThT-complexed self-assembled beta-sheet structures; FIG. 19 shows CD spectrum of peptide 1 after incubation for two weeks with legumain; and FIG. 20 is a graph showing the fluorescence intensity enhancement of ThT at different legumain concentrations.

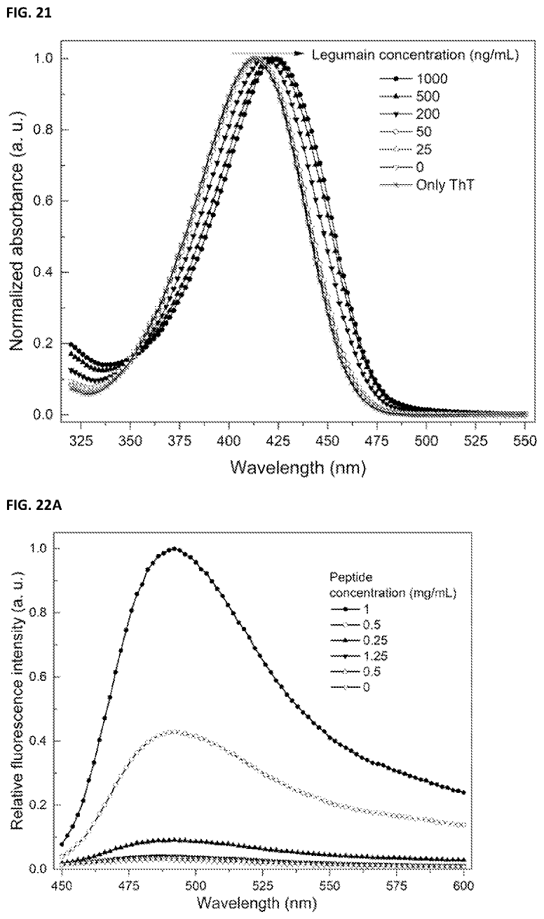

FIG. 21 shows representative absorbance spectra of ThT in the presence of peptide 1 after a two hour incubation with different amounts of legumain.

FIG. 22A is a line graph showing representative fluorescence spectra of ThT in the presence or absence of peptide 1 after incubation with legumain; and, FIG. 22B is a bar graph showing the fluorescence intensity enhancement of ThT at different legumain concentrations with or without legumain.

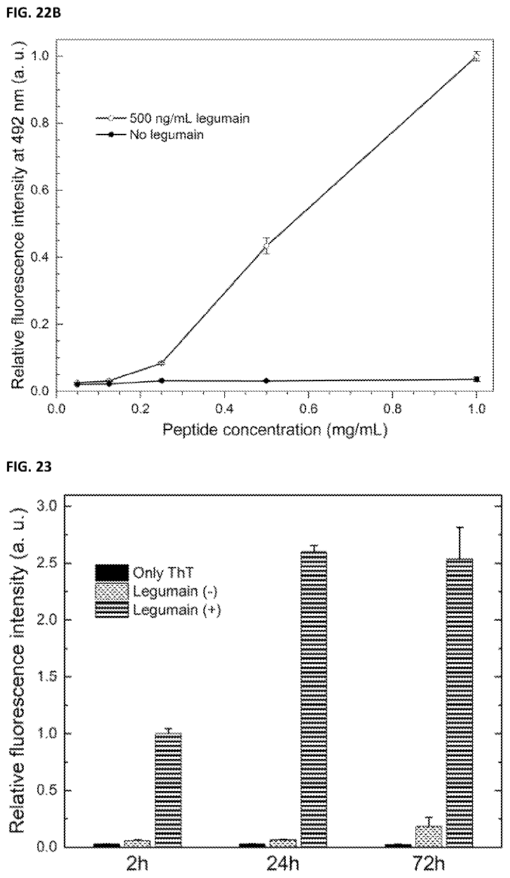

FIG. 23 is a bar graph showing the relative fluorescence intensity of ThT in peptide 1 solution with or without legumain at different time points.

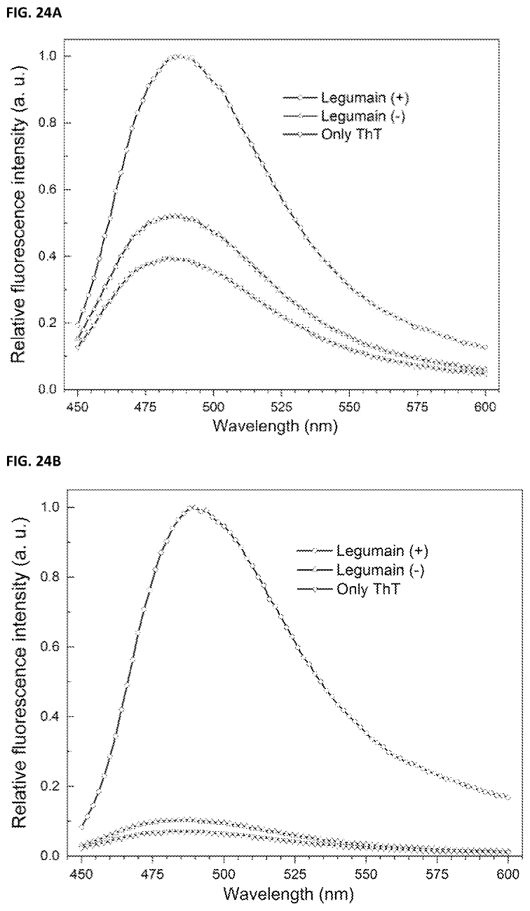

FIGS. 24A and 24B are representative fluorescence spectra of ThT in, respectively, 20% plasma and albumin depleted 20% plasma, in the presence or absence of peptide 1 after incubation with or without legumain.

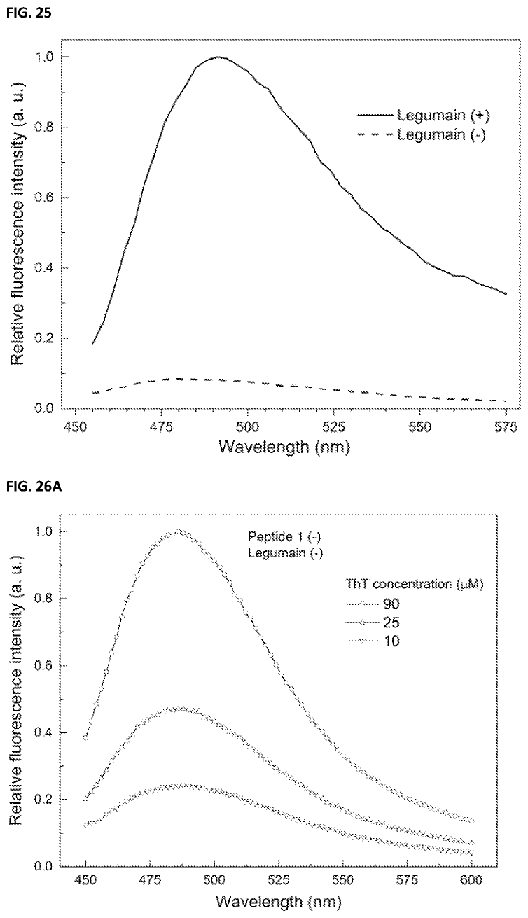

FIG. 25 is a fluorescence spectra of peptide 1 samples incubated in 10% plasma in the presence or absence of Legumain after separating the peptide aggregates.

FIGS. 26A and 26B are line graphs showing the fluorescence of ThT at different concentrations in 10% plasma, respectively, without peptide1 or legumain and with peptide 1 and legumain.

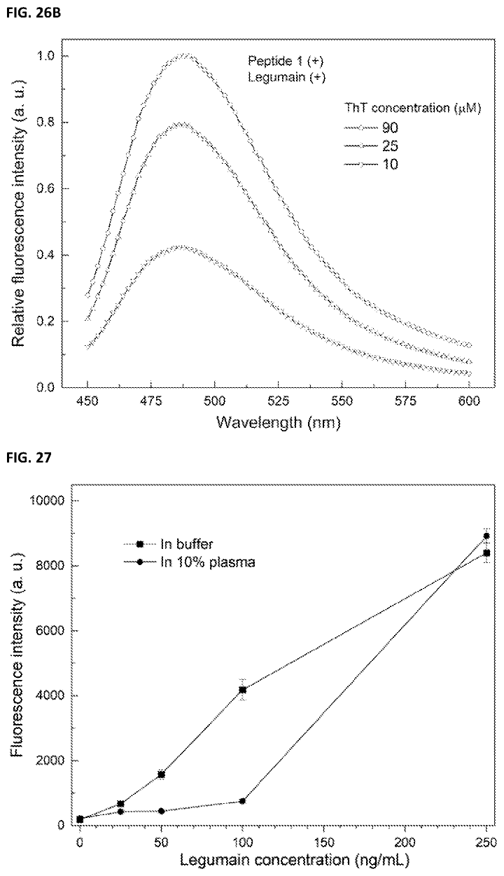

FIG. 27 is a line graph of a Z-AAN-AMC probe after incubation with different amounts of legumain in buffer or 10% plasma.

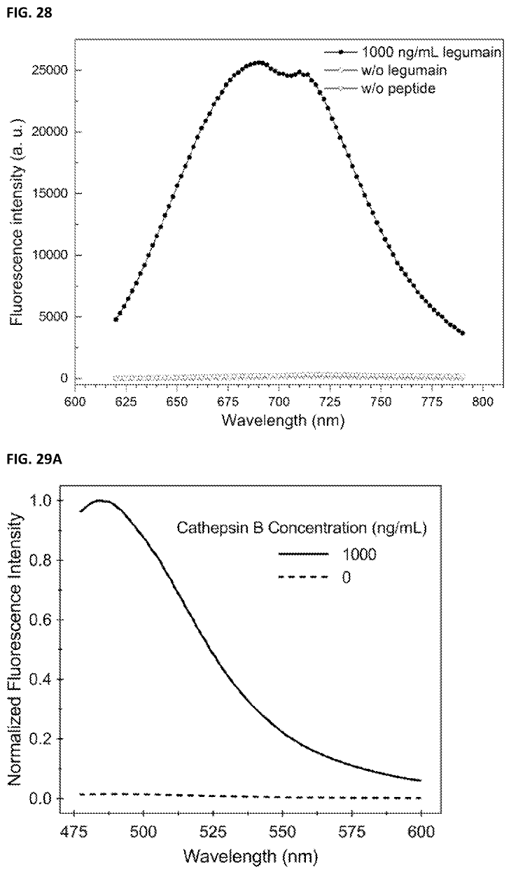

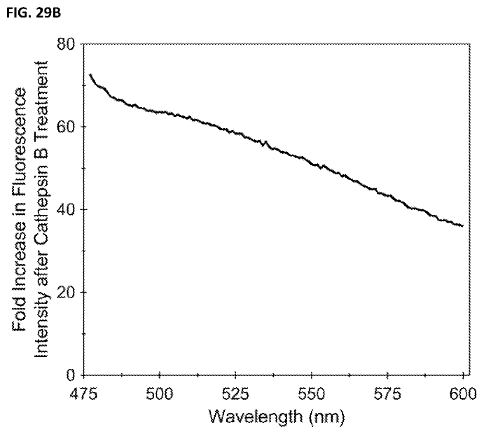

FIG. 28 is a line graph of representative fluorescence spectra of MCAAD-3 in the presence or absence of peptide 1 with different amounts of legumain.

FIG. 29A shows the representative fluorescence spectra of ThT after treating peptide 3 with 1000 ng/mL and 0 ng/mL cathepsin B. FIG. 29B shows the fold-increase in ThT fluorescence intensity after treatment of peptide 3 with 1000 ng/mL as compared to untreated peptide 3 (0 ng/mL cathepsin B).

FIGS. 1A, 1B, and 1C show how, in an exemplary embodiment, peptide 1 was designed to develop β-sheet structure upon hydrolysis by the protease of interest. As shown in FIGS. 1A, 1B, and 1C, the peptide is composed of three elements: a β-strand motif, a protease substrate motif, and a hydrophilic motif to solubilize the probes and prevent their self-assembly in the absence of protease activity. The protease substrate motif cleavage by the protease of interest and release of the hydrophilic motif triggers the formation of β-sheet containing self-assembled structures. ThT, which is commonly used to stain amyloid fibers²⁶⁻³⁰ or other β-sheet structures^{31,32} due to its large fluorescence enhancement upon binding to β-sheet structures, was used to detect the self-assembled structures formed in response to protease activity (Kelly, S. M. et al., How to study proteins by circular dichroism. Proteomics 2005, 1751 (2), 119-139; Greenfield, N.J., Using circular dichroism spectra to estimate protein secondary structure. Nat. Protoc. 2006, 1 (6), 2876-2890). Another amyloid dye, MCAAD-3, was used to label the self-assembled structures (Micsonai, A. et al., Accurate secondary structure prediction and fold recognition for circular dichroism spectroscopy. Proc. Natl. Acad. Sci. 2015, 112 (24), E3095-E3103); Micsonai, A. et al., BeStSeI: a web server for accurate protein secondary structure prediction and fold recognition from the circular dichroism spectra. Nucleic Acids Res. 2018, 46 (W1), W315-W322).

The exemplary method described herein is label-free and, thus, no chemical synthesis or bioconjugation reaction is required. This novel assay consists of a commercially obtainable β-sheet forming peptides without any special modification and intercalating dyes such as Thioflavin T (ThT).

Most quenching based probes developed for monitoring the activity of proteases suffer from incomplete quenching of the fluorophores, which yields a high background signal and low enhancement in the signal upon hydrolysis of the probes by the protease of interest. The high background signal makes the accurate detection of low protease levels challenging and diminish the sensitivity and selectivity of these probes.

In the absence of the target protease the self-assembling polypeptides disclosed herein demonstrated very low background signal with high signal on/off ratios (>30) (See FIGS. 9A, 9B, 10, and 11).

As disclosed herein, it was demonstrated that the exemplary method can be used to detect protease activity in complex biological environments such as human plasma.

Internally quenched peptide substrates: Skilled persons in the art will know that there are two types of such reporters.¹⁻³ In the first type, the fluorescence of the dye attached to the peptide substrate is quenched by the internal energy transfer between the peptide and the dye. Upon peptide cleavage by the protease, the fluorescence of the dye is recovered. In this design, the dye should be attached to the P1′ position of the substrate. Therefore, such probes cannot be used for all types of proteases as some proteases cleave very specific substrates and are sensitive to the amino acids at P′ positions, especially the P1′ position. In the second probe type, the fluorescence of the dye is quenched by a suitable quencher molecule. In this design, the fluorophore does not have to be attached to the P1′ position. The fluorophore and the quencher are usually attached to the opposite ends of the peptide, and the fluorescence of the dye is quenched through fluorescence resonance energy transfer (FRET). The main limitation of this approach is the incomplete quenching of the fluorophore, which generates a high background signal. For both types of probes, peptide substrates should be conjugated with fluorescent labels through organic synthesis or bioconjugation reactions, which is costly and requires time-consuming purification steps.

In contrast, the exemplary method disclosed herein consists of only two commercially available components; i) a self-assembling polypeptide and ii) a ß-sheet intercalating dye, and no chemical synthesis is required. As the ß-sheet intercalating dyes have a very weak emission in the free form, the method's background signal is low, and high ON/OFF ratios (>100) can be achieved. Both types of internally quenched peptide substrates were designed for a myriad of proteases, and they are commercially available from many companies (e.g., Invitrogen North America, Bachem, PerkinElmer, Abcam).

Dual fluorescence quenched probes: In a few studies,⁹⁻¹¹ peptide self-assembly was combined with the internal quenching strategies to better quench the fluorophores through both internal energy transfer and aggregation-induced quenching. While in these studies, a better quenching (i.e., lower background signal) was achieved, the design and synthesis of these probes are even more complicated than the probes mentioned above.

Nanomaterial based fluorescence quenching: Another common approach in the literature is to use nanomaterials⁴⁹ such as quantum dots,^{8,50} gold nanoparticles,⁵¹ or graphene oxide^{4,52} to quench the fluorescence of the dye, which is attached to the nanoparticle surface using a peptide substrate that can be cleaved by the protease of interest. Like the probes mentioned above, the quenching is inefficient, with a high background signal for most of these probes. In addition, the use of nanomaterials complicates the synthesis and brings reproducibility issues. Also, some of these nanomaterials, such as graphene and quantum dots, are toxic.

Charge-changing peptides: These probes can be used to detect protease activity directly in whole blood or plasma.⁵³⁻⁵⁵ However, the reporter should be separated from the sample at the last step of the assay using gel electrophoresis, which is a low-throughput and time-consuming process.

### EXAMPLES

The following examples are for illustration only. In light of this disclosure, those of skill in the art will recognize that variations of these examples and other embodiments of the disclosed subject matter are enabled without undue experimentation.

### Example 1—Enzyme-Instructed Formation of Beta-Sheet Rich Nanoplatelets for Label-Free Protease Sensing

Dysregulated proteolytic activity has been observed in various human diseases, including cancer, neurodegenerative disorders, and cardiovascular diseases. Thus, there is an immense need to develop simple and sensitive methods to monitor specific protease activities in biological solutions for the detection and prognosis of these diseases. Disclosed herein is a fluorogenic label-free protease detection method using a rationally designed β-sheet rich nanoplatelet forming peptide precursor and a β-sheet intercalating dye: Thioflavin T. Hydrolysis of the peptide by the target protease triggers the formation of β-sheet rich self-assembled, 3 nanometer thick nanoplatelets. In situ intercalation of Thioflavin T into these β-sheet domains resulted in significant enhancement in the dye's fluorescence, allowing sensitive detection of protease activity with high signal-to-noise ratios (up to 45 fold). The concept was demonstrated to detect the activity of legumain, a cysteine protease that was found to be over-expressed in several cancers, with a detection limit of about 0.2 nM. In addition, assay conditions were optimized to detect legumain activity in human plasma. Importantly, both assay components can be commercially obtained, and no time-consuming conjugation reactions and purification steps are required. Thus, the method described herein may be utilized in various protease detection applications, with its simplicity and low cost.

Proteases, which catalyze peptide bond hydrolysis, form a large enzyme family encompassing ˜600 proteins in humans (i.e., ˜2% of the human proteome) (Puente, X. S.; Sánchez, L. M.; Overall, C. M.; López-Otin, C. Human and Mouse Proteases: A Comparative Genomic Approach. Nat. Rev. Genet. 2003, 4 (7), 544-558; Dudani, J. S.; Warren, A. D.; Bhatia, S. N. Harnessing Protease Activity to Improve Cancer Care. Annu. Rev. Cancer Biol. 2018, 2 (1), 353-376). Together with their endogenous inhibitors, protease activity plays a critical role in many biological processes such as apoptosis, digestion, coagulation, cell migration, wound healing, and immunity (López-Otin, C.; Bond, J. S. Proteases: Multifunctional Enzymes in Life and Disease. J. Biol. Chem. 2008, 283 (45), 30433-30437). Dysregulated proteolytic activity has been observed in a variety of human diseases, including cancer, neurodegenerative disorders, and cardiovascular diseases, to name a few (López-Otin, C.; Bond, J. S. Proteases: Multifunctional Enzymes in Life and Disease. J. Biol. Chem. 2008, 283 (45), 30433-30437; Olson, O. C.; Joyce, J. A. Cysteine Cathepsin Proteases: Regulators of Cancer Progression and Therapeutic Response. Nat. Rev. Cancer 2015, 15 (12), 712-729; Mason, S. D.; Joyce, J. A. Proteolytic Networks in Cancer. Trends Cell Biol. 2011, 21 (4), 228-237)) In cancer, aberrant protease activity is associated with tumor progression, invasion, and metastasis, as well as immune suppression and drug resistance (Mason, S. D.; Joyce, J. A. Proteolytic Networks in Cancer. Trends Cell Biol. 2011, 21 (4), 228-237). Thus, there is a growing interest in developing new assays and/or medical imaging methods to monitor specific protease activities for detection and prognosis of cancer and other diseases (Dudani, J. S.; Warren, A. D., Bhatia, S. N., Harnessing Protease Activity to Improve Cancer Care. Annu. Rev. Cancer Biol. 2018, 2 (1), 353-376; Oliveira-Silva, R.; Sousa-Jerónimo, M.; Botequim, D.; Silva, N. J. O.; Paulo, P. M. R., Prazeres, D. M. F. Monitoring Proteolytic Activity in Real Time: A New World of Opportunities for Biosensors. Trends Biochem. Sci. 2020, 45 (7), 604-618). Indeed, currently deployed methods are finding utility in protease-targeted therapeutic development for the identification of inhibitors, and could be useful for assessing response to treatment (Turk, B. Targeting Proteases: Successes, Failures and Future Prospects. Nat. Rev. Drug Discov. 2006, 5 (9), 785-799). Over the past few decades, various assays have been developed to detect protease activity, with the most widely reported ones using quenched probes (Poreba, M. et al. Small Molecule Active Site Directed Tools for Studying Human Caspases. Chem. Rev. 2015, 115 (22), 12546-12629; Ong, I. L. H. and Yang, K. L., Recent Developments in Protease Activity Assays and Sensors. Analyst 2017, 142 (11), 1867-1881). In this detection scheme, a fluorophore is attached to a protease substrate where its emission is typically quenched by internal energy transfer to the peptide substrate, a quencher molecule, or a nanoparticle (Edgington, L. E. et al., Functional Imaging of Legumain in Cancer Using a New Quenched Activity-Based Probe. J. Am. Chem. Soc. 2013, 135 (1), 174-182; Shi, L. et al., Synthesis and Application of Quantum Dots FRET-Based Protease Sensors. J. Am. Chem. Soc. 2006, 128 (32), 10378-10379; Craven, T. H. et al., Super-silent FRET Sensor Enables Live Cell Imaging and Flow Cytometric Stratification of Intracellular Serine Protease Activity in Neutrophils. Sci. Rep. 2018, 8 (1), 13490; Medintz, I. L. et al., Proteolytic Activity Monitored by Fluorescence Resonance Energy Transfer through Quantum-Dot-Peptide Conjugates. Nat. Mater. 2006, 5 (7), 581-589; Zhang, M. et al., Interaction of Peptides with Graphene Oxide and Its Application for Real-Time Monitoring of Protease Activity. Chem. Commun. 2011, 47 (8), 2399-2401; Jiang, Y. et al., Huang, Y. Molecular-Dynamics-Simulation-Driven Design of a Protease-Responsive Probe for In-Vivo Tumor Imaging. Adv. Mater. 2014, 26 (48), 8174-8178; Lee, S. et al., A Near-Infrared-Fluorescence-Quenched Gold-Nanoparticle Imaging Probe for In Vivo Drug Screening and Protease Activity Determination. Angew. Chemie Int. Ed. 2008, 47 (15), 2804-2807). The hydrolysis of the peptide substrate by the target protease separates the fluorophore and its quencher and restores the fluorescence of the probe. These probes often suffer from high background signal due to the incomplete quenching of the dyes and, thus, low signal enhancement after protease cleavage (<10) is typically obtained.

Recent advances in the understanding of the properties of self-assembling peptide structures has enabled application of this concept to protease activity sensing. For instance, incorporating self-assembly motifs to conventional quenched probes can lower their background signal by further quenching fluorophore emission through aggregation-induced quenching (Wei, G. et al., Self-Assembling Peptide and Protein Amyloids: From Structure to Tailored Function in Nanotechnology. Chem. Soc. Rev. 2017, 46 (15), 4661-4708; Zhang, W. et al., Protein-Mimetic Peptide Nanofibers: Motif Design, Self-Assembly Synthesis, and Sequence-Specific Biomedical Applications. Prog. Polym. Sci. 2018, 80, 94-124; Levin, A. et al., Biomimetic Peptide Self-Assembly for Functional Materials. Nat. Rev. Chem. 2020, 4 (11), 615-634; Ren, C.; Wang, H. et al., When Molecular Probes Meet Self-Assembly: An Enhanced Quenching Effect. Angew. Chemie-Int. Ed. 2015, 54 (16), 4823-4827; Lock, L. L. et al., Design and Construction of Supramolecular Nanobeacons for Enzyme Detection. ACS Nano 2013, 7 (6), 4924-4932). The utilization of peptide self-assembly also offers new opportunities to design molecular probes for more sensitive detection of protease activity. For example, enzyme-instructed self-assembly (EISA) of peptides conjugated to an aggregation-induced emission dye can enable the development of bright turn-on probes with high ON/OFF ratios (Zhao, Y. et al., Spatiotemporally Controllable Peptide-Based Nanoassembly in Single Living Cells for a Biological Self-Portrait. Adv. Mater. 2017, 29 (32), 1601128; Shi, H. et al., Real-Time Monitoring of Cell Apoptosis and Drug Screening Using Fluorescent Light-up Probe with Aggregation-Induced Emission Characteristics. J. Am. Chem. Soc. 2012, 134 (43), 17972-17981; Han, A. et al., Peptide-Induced AlEgen Self-Assembly: A New Strategy to Realize Highly Sensitive Fluorescent Light-Up Probes. Anal. Chem. 2016, 88 (7), 3872-3878). In recent years, EISA has also been applied to develop probes for other imaging modalities such as photoacoustic or magnetic resonance imaging (Dragulescu-Andrasi, A. et al., Activatable Oligomerizable Imaging Agents for Photoacoustic Imaging of Furin-like Activity in Living Subjects. J. Am. Chem. Soc. 2013, 135 (30), 11015-11022; Wu, C., Alkaline Phosphatase-Triggered Self-Assembly of Near-Infrared Nanoparticles for the Enhanced Photoacoustic Imaging of Tumors. Nano Lett. 2018, 18 (12), 7749-7754; Yuan, Y. et al., Intracellular Self-Assembly and Disassembly of 19F Nanoparticles Confer Respective “off” and “on” 19F NMR/MRI Signals for Legumain Activity Detection in Zebrafish. ACS Nano 2015, 9 (5), 5117-5124). However, previously developed EISA or quenching-based protease activity assays require labeling the pro-tease substrates with a fluorophore or other type of molecular probe, which complicates their synthesis and increases their cost. Thus, the development of label-free methods for sensitive detection of protease activity is still of great importance.

Disclosed herein is a distinct EISA-based method, namely enzyme-instructed β-sheet formation, for label-free and turn-on fluorescent detection of protease activity. The method utilizes a commercially obtainable polypeptide without any special modification and a cost-effective intercalating dye, Thioflavin T (ThT). As disclosed herein, Peptide 1 was designed to develop β-sheet structure upon hydrolysis by the protease of interest, peptide (peptide 1) shown in FIGS. 1A through 1D. Peptide 1 was to designed to composed three elements: a β-sheet forming motif, a protease substrate, and a hydrophilic motif to solubilize the probes and prevent their self-assembly in the absence of protease activity. The protease substrate motif cleavage by the protease of interest releases the hydrophilic motif and triggers the formation of β-sheet rich 3 nm thick self-assembled nano-platelets.

ThT, which is commonly used to stain amyloid fibers due to its large fluorescence enhancement upon binding to β-sheet domains, was used to detect the self-assembled nanoplatelets formed in response to protease activity. In this proof-of-concept study, we developed an assay using the methods disclosed herein to detect legumain activity, a cysteine protease that was found to be over-expressed in several cancers (Levine, H. Thioflavine T Interaction with Synthetic Alzheimer's Disease B-amyloid Peptides: Detection of Amyloid Aggregation in Solution. Protein Sci. 1993, 2 (3), 404-410; Sulatskaya, A. I. et al., Fluorescence Quantum Yield of Thioflavin T in Rigid Isotropic Solution and Incorporated into the Amyloid Fibrils. PLoS One 2010, 5 (10), e15385; Liu, C. et al., Overexpression of Legumain in Tumors Is Significant for Invasion/Metastasis and a Candidate Enzymatic Target for Prodrug Therapy. Cancer Res. 2003, 63 (11), 2957-2964). In some embodiments, the disclosed method may be applied to other proteases by selecting a protease substrate motif that comprises a protease cleavage site of a desired protease.

### a) Experimental Methodology

Materials. Peptide 1 (SEQ ID NO: 206) (1822.8 g/mol) and peptide 2 (SEQ ID NO: 207) (1048.2 g/mol) were purchased from GenScript and used as received (Genscipt USA Inc. 860 Centennial Ave. Piscataway, NJ 08854, USA). Recombinant mouse legumain was obtained from Novus Biologicals (Novus Biologicals, LLC, 10730 E. Briarwood Avenue, Building IV, Centennial, CO 80112, USA). Thioflavin T was purchased from Santa Cruz Biotechnology, 2145 Delaware Avenue, Santa Cruz CA, 95060, USA). Legumain inhibitor, RR-11a analog, was purchased from MedChemExpress. Z-AAN-AMC was purchased from Bachem (Bachem Americas, Inc., 3132 Kashiwa Street Torrance, CA 90505, USA). Pierce™ albumin depletion kit was purchased from Thermo Scientific (Thermo Fisher Scientific. 168 Third Avenue. Waltham, MA 02451, USA). Human plasma was obtained from Innovative Research, Inc (Innovative Research, Inc, 46430 Peary Ct., Novi, Michigan, 48377, USA).

Legumain activation. To activate legumain, 5 μL of prolegumain solution (0.5 mg/mL in Tris buffer containing 10% glycerol) was mixed with 20 μL of activation buffer (50 mM Sodium Acetate, 100 mM NaCl, pH 4.0) and incubated at 37° C. for 2 h. It was then diluted in 225 μL of legumain assay buffer (50 mM MES, 250 mM NaCl, pH 5) to give a final legumain concentration of 10 μg/mL and immediately used in the assay.

Legumain assay. In a typical assay, peptide 1 was first dissolved in ultrapure water containing 25% DMSO at a peptide concentration of 10 mg/mL. It was then diluted in phosphate-buffered saline (PBS, pH 7.4, 10 mM) to give a peptide concentration of 2 mg/mL. Next, 50 μL of the peptide solution was mixed with 50 μL of MES buffer (50 mM MES, 250 mM NaCl, pH 5) containing activated legumain at different concentrations (0-2000 ng/mL) in a 96 well plate and the plate was incubated at 37° C. for 2 h. Note that the final peptide concentration was 1 mg/mL and final legumain concentrations were between 0 and 1000 ng/mL. Finally, 10 μL of ThT solution (1 mM, in ultrapure water) was added to each well, and ThT fluorescence was measured using a Spark 20M microplate reader (Tecan) after 15-30 min incubation at room temperature.

In the peptide concentration experiment, appropriate amounts of peptide 1 stock solution (10 mg/mL) were diluted in PBS and mixed with the MES buffer containing legumain, as described above, to give the final peptide concentrations between 0.05 and 1 mg/mL in the assay. In kinetic studies, ThT solution was mixed with the peptide immediately before the addition of activated legumain, and the plate was incubated in a Spark 20M microplate reader (Tecan) at 37° C. for 3 hours, and ThT fluorescence was recorded every 4 minutes. For the inhibitor experiment, the legumain inhibitor, RR-11a, was first dissolved in DMSO (1 mM), and appropriate amounts of inhibitor were incubated with legumain for about 1.0 hour at room temperature in 96 well plates. Finally, the legumain solutions incubated with different amounts of inhibitor were mixed with the peptide 1 solution, and the assay was performed as described above. For the experiments in plasma, 10 μL or 20 μL of PBS in the wells were replaced with human plasma to achieve final plasma concentrations of 10% and 20%, respectively.

Legumain assay with the commercial probe. The commercial legumain probe (Z-AAN-AMC) was dissolved in DMSO to give a peptide concentration of 1.0 mM. 2.5 μL of probe solution was mixed with 47.5 μL of PBS (10 mM, pH 7.4 MES buffer and 50 μL of MES buffer (50 mM MES, 250 mM NaCl, pH 5) containing different amounts of activated legumain in a 96 well plate and the plate was incubated at 37° C. for about 2.0 hours. The fluorescence of the AMC dye was measured using a Spark 20M microplate reader (Tecan). For the experiments in plasma, 10 μL of PBS was replaced with plasma to achieve final plasma concentration of 10%.

Transmission electron microscopy (TEM) and atomic force microscopy (AFM) imaging. For TEM and AFM measurements, peptide 2 was first dissolved in DMSO to give a peptide concentration of 5.8 mg/mL and diluted in the assay buffer used in the legumain cleavage experiments (44% PBS+56% MES, see above for details) to give a final concentration of 0.58 mg/mL. After incubating at 37° C. for 2.0 hours, the formed aggregates were collected by centrifugation and resuspended in ultrapure water. Peptide 1 was first dissolved in ultrapure water containing 25% DMSO to give a peptide concentration of about 10 mg/mL and diluted in the assay buffer to give a final peptide concentration of about 1.0 mg/mL and incubated with legumain (1000 ng/mL) at 37° C. for about 2.0 hours. Peptide 1 aggregates were also collected by centrifugation and resuspended in ultrapure water. To separate large aggregates, the peptide 1 solution was bath sonicated for 30 minutes just before sample preparation. TEM images were taken using a Tecnai microscope (FEI). To prepare TEM samples, 5 μL of solutions were placed on carbon film 200 copper mesh TEM grids. Samples were incubated on TEM grids for about 5 minutes and bloated and air dried. Uranyl acetate was prepared in distilled water at 2% w/v and filtered with a 0.1 μm syringe filter before each use. A 20 μl droplet of this solution was placed on Parafilm and the TEM grid was floated on it for 7 minutes. Excess uranyl acetate was blotted using Whatman paper, and the sample is left to dry at room temperature.

AFM imaging was performed with Peakforce-HiRs-F-B probes on a Fastscan scanner of a Dimension Fastscan Bio system (Bruker Nano Surfaces). Positively charged surfaces were prepared by incubating 0.01% aqueous poly-L-ornithine (PLO) on freshly cleaved 9.9 mm mica discs (Ted Pella, Inc.), rinsing with ultrapure water, drying under a stream of nitrogen, and vacuum desiccating overnight. The peptide 1 and 2 solutions were further diluted 2.5× in ultrapure water and bath sonicated for 30 minutes in Protein LoBind Eppendorf tubes. Without sonication, the self-assembled peptide nanoparticles aggregated into particles microns to millimeters in size, which were incompatible with the vertical scan range of the AFM. Onto the PLO-mica surfaces, 20 μL of the respective sonicated samples were added. After 30 min, the surface was gently rinsed 2× with 100 μL ultrapure water, loaded into the AFM, and thermally equilibrated with 100 μL ultrapure water for about 45 minutes to reduce noise. Imaging was immediately performed in tapping mode with a minimum resolution of 512×512, and scan speeds inversely proportional to the scan size. Data were processed and analyzed in Nanoscope Analysis 2.0 (Bruker Nano Surfaces).

Circular dichroism (CD) Measurements. CD measurements were performed on a J-1500 circular dichromator (JASCO, Inc.) using 1.0 mm, stoppered Suprasil quartz cuvettes (Hellma). Peptide 2 was dissolved at 0.5 mg/mL in Protein LoBind Eppendorf tubes with ultrapure water adjusted to pH 9.5 with 10 N NaOH and then diluted to 0.35 mg/mL with low far-UV absorbance CD buffer (final concentration: 10 mM NaH2PO4, 137 mM NaF, 2.7 mM KF).^{31,32} Spectra were acquired from 330-180 nm at 21° C. with 1 nm bandwidth, 10 nm/min scan speed, and 4 sec integration time. A series of 13 sample scans were averaged, background corrected with buffer blank spectra, and smoothed with a Savitzky-Golay filter. The Beta Structure Selection (BeStSel) method^{33,34} was used for secondary structure estimation (SSE) of peptide 2. SSE was performed on the BeStSel webserver hosted by Eötvös Lorend University³⁴ using spectral data from 180-250 nm.

A time-course study of the legumain assay was also run. Peptide 1 was dissolved at 0.333 mg/mL in low far-UV absorbance CD buffer, pH 5.5-6. Legumain was activated for abut 2 hours at 37° C. in far-UV absorbance CD buffer, pH 4. Spectra were acquired from 260-190 nm with 1.0 nm band-width, 20 nm/min scan speed, and 2 sec integration time. A 5 minute acquisition cycle was automatically run 26 times, followed by manual acquisitions at 28 hours, 53 hours, 78 hours, and 14 days. The temperature was maintained at 37° C. throughout. Legumain (333 ng/mL) was mixed with peptide 1 just prior to acquisition of the second spectrum (the 0 min time point). All spectra were subsequently background subtracted and then smoothed using a Savitsky-Golay filter. Data are presented in units of molar circular dichroism, Δε(M⁻¹ cm⁻¹).

Fourier-transform infrared spectroscopy (FTIR) measurements. FTIR measurements were performed on a Nicolet iS5 KBR window FTIR (Thermo Fisher Scientific. 168 Third Avenue. Waltham, MA 02451, USA) with an iD7 anti-reflectance diamond crystal attenuated total reflectance (ATR) module. Peptide 1 and peptide 2 were prepared at 10 mg/mL in D2O (≥99.8% D, Acros Or-ganics) with 25% anhydrous DMSO (≥99.9%, Sigma AI-drich) adjusted to pD 6.5 with 10 mM NaOD (≥99.0% D, Acros Organics) and then diluted to about 1.0 mg/mL in D2O, pD 6.5. For peptide 1 with legumain, the assay was performed as described with about 1.0 mg/mL peptide 1 and about 2000 ng/mL legumain in about 1 mL total volume. The self-assembled aggregates were pelleted by centrifugation at about 21,000×g for 30 minutes, the supernatant was replaced with D2O, pD 6.5, and the pellet was partially resuspended by vortexing. This process was repeated 3 times to prevent the ˜1640 cm⁻¹ water bending peak from obscuring the amide I secondary structural fingerprint of the peptide aggregates. Deuterated water was required as aqueous buffers resulted in intense water peaks even after drying, which was likely due to trapped water in the peptide film. The pellet was diluted in D2O, pD 6.5, to approximately 1.0 mg/mL peptide 2 content as determined by Fmoc absorbance at 301 nm on a Cary 3500 UV-Vis spectrophotometer (Agilent Technologies, Inc.). For each sample, about 2.0 μL of about 1.0 mg/mL peptide content was deposited directly onto the diamond ATR crystal, dried under a stream of clean dry air, scanned 512 times at 2 cm⁻¹ resolution from 4000-400 cm⁻¹ under a stream of clean dry air, background subtracted using dried sample-matched buffer, and auto baseline corrected in OMNIC 9.2 software. Data from 1800-1500 cm⁻¹ are reported.

Fluorescence spectroscopy. For fluorescence measurements, peptide 2 was dissolved in DMSO to give a peptide concentration of 10 mg/mL, and it was 5× diluted in DMSO or the assay buffer and fluorescence spectra of the Fmoc groups were recorded using an FP-8500 spectrofluorometer (JASCO, Inc). Liquid chromatography mass spectrometry (LC-MS) measurements. LC-MS measurements were carried using an Acquity UPLC System (Waters) equipped with a SQ Detector 2 (Waters) and a C18 column (Waters). For LC-MS measurement, peptide 1 was first dissolved in ultrapure water containing 25% DMSO and diluted in PBS and MES mixture with or without legumain as described above. Final peptide concentration was about 0.5 mg/mL and legumain concentrations were about 0 ng/mL and about 1000 ng/mL. Samples were incubated at 37° C. for about 2 hours, diluted in HPLC grade water and acetonitrile mixture (1:1) containing 1% formic acid, and loaded to the column.

β-sheet rich nanoplatelet formation by self-assembly of peptide 2. To test the hypothesis, Peptide 2 was used as shown in FIG. 1B, which is composed of the β-strand motif (Fmoc-FKFE) and the portion of the legumain substrate that remains attached to the self-assembly motif upon hydrolysis of peptide 1 (Smith, A. M. et al., Fmoc-Diphenylalanine Self Assembles to a Hydrogel via a Novel Architecture Based on π-π Interlocked β-Sheets. Adv. Mater. 2008, 20 (1), 37-41; Bowerman, C. J. and Nilsson, B. L., A Reductive Trigger for Peptide Self-Assembly and Hydrogelation. J. Am. Chem. Soc. 2010, 132 (28), 9526-9527; He, X. et al., Inflammatory Monocytes Loading Protease-Sensitive Nanoparticles Enable Lung Metastasis Targeting and Intelligent Drug Release for Anti-Metastasis Therapy. Nano Lett. 2017, 17 (9), 5546-5554). As peptide 2 is not soluble in aqueous solutions, it was first dissolved in DMSO and diluted in assay buffer (supporting information is disclosed herein) to induce the aggregation of peptide 2 (0.58 mg/mL) and formation of β-sheet structures. ThT (90 μM) addition to this solution yielded a bright fluorescence with an emission maximum of about 490 nm (see FIG. 2). A 45-fold enhancement in the ThT fluorescence intensity was detected in the presence of peptide 2, suggesting the intercalation of ThT into the self-assembled structures of peptide 2 (Brahmachari, S. et al., Diphenylalanine as a Reductionist Model for the Mechanistic Characterization of β-Amyloid Modulators. ACS Nano 2017, 11 (6), 5960-5969).

It was observed that the self-assembled structures formed by peptide 2 could be collected after brief centrifugation (see FIGS. 1A, 1B, and 1C). The morphology of these structures was investigated using transmission electron microscopy (TEM) and atomic force microscopy (AFM). TEM showed the formation of micron-sized aggregates of smaller platelets with sizes from tens to hundreds of nanometers (FIGS. 3A and 3B). Interestingly, nano-platelets with both regular (short rod and triangular) and irregular shapes were observed (see FIG. 3B). AFM experiments were performed to further analyze the morphology of self-assembled nano-platelets. Before AFM imaging, peptide 2 solution was bath sonicated to break up the large aggregates, which facilitated high-resolution imaging of the plate structures. FIGS. 4A and 4B show the representative AFM images of the sonicated peptide 2 sample, which also revealed the formation of similar nanoplatelet structures with a thickness of about 3 nm. The number of regularly shaped platelets was reduced in the AFM images, which was most likely due to the reorganization of the peptide aggregates during the bath sonication process. The TEM and AFM results suggest that the peptide assembly formed nanoplatelets with a high degree of molecular organization. It was noted that similar structures were reported before for β-sheet forming Fmoc modified short peptides (Smith, A. M. et al, Fmoc-Diphenylalanine Self Assembles to a Hydrogel via a Novel Architecture Based on π-π Interlocked β-Sheets. Adv. Mater. 2008, 20 (1), 37-41; Williams, R. J. et al., Enzyme-Assisted Self-Assembly under Thermodynamic Control. Nat. Nanotechnol. 2009, 4 (1), 19-24).

Circular dichroism (CD) was used to investigate the molecular orientation of peptide 2 in the self-assembled structures (As shown in FIG. 5A). A negative peak at about 218 nm was detected in the CD spectrum of peptide 2, which indicated the formation of β-sheet structures (Smith, A. M. et al., Fmoc-Diphenylalanine Self Assembles to a Hydrogel via a Novel Architecture Based on π-π Interlocked β-Sheets. Adv. Mater. 2008, 20 (1), 37-41). Another negative peak about 195 nm was also observed, which suggests the presence of random coil structure. Structural analysis of the CD data estimated that peptide 2 aggregates are composed of approximately 45% anti-parallel β-sheet structures to which ThT can bind (FIG. 5B). To further confirm the formation of β-sheet structures by peptide 2, we performed Fourier-transform infrared spectroscopy (FTIR) measurements. FTIR spectrum of peptide 2 (See FIG. 13) showed an intense peak at 1624 cm⁻¹, which indicates major β-sheet structure content, and an accompanying high frequency peak at 1688 cm⁻¹ suggests an anti-parallel orientation (Smith A. M. et al, 2008). In addition, a moderately intense peak at 1643 cm⁻¹ and a lower intensity shoulder peak at 1660 cm⁻¹ were also observed and can be assigned to random coil and α-helix structure, respectively (Kong, J. and Yu, S., Fourier Transform Infrared Spectroscopic Analysis of Protein Secondary Structures. Acta Biochim. Biophys. Sin. (Shanghai). 2007, 39 (8), 549-559). A broad shoulder peak in the 1668-1683 cm-1 region suggests some β-turn content. In accordance with the CD observations, FTIR measurements of peptide 2 suggest that a mixture of molecular organizations was present in the nanoplatelets with predominant random coil and β-sheet content. The molecular structure of peptide 2 was also studied using fluorescence spectroscopy (See FIG. 14). The emission spectra of Fmoc groups were recorded for peptide 2 dissolved in DMSO or buffer. In DMSO, where the peptide is soluble, only the Fmoc monomer emission peak was detected at 307 nm (Smith A. M. et al., 2008). Interestingly, a shoulder peak of the monomer peak around 314 nm was also observed, suggesting intermolecular interactions between peptide 2 molecules. Nevertheless, the monomer peak was narrow and intense, as expected for solubilized Fmoc modified small peptides. In buffer, the intensity of the monomer peak was decreased significantly (about 12 fold) compared to the peak intensity in DMSO due to the aggregation of peptide 2 (He, X. et al., Inflammatory Monocytes Loading Protease-Sensitive Nanoparticles Enable Lung Metastasis Targeting and Intelligent Drug Release for Anti-Metastasis Therapy. Nano Lett. 2017, 17 (9), 5546-5554). The monomer peak was significantly broadened and red-shifted to about 328 nm, which also suggests aggregation of the peptide. An additional weak and broad emission peak of about 440 nm, corresponding to the Fmoc excimers, was detected. This indicates a β-sheet structure arrangement in which Fmoc molecules can form excimers through π-stacking (Smith A. M. et al., 2008; Pinion, J. P. et al., Excimer Emission from Dibenzofuran and Substituted Fluorenes. J. Lumin. 1971, 3 (4), 245-252).

Finally, molecular simulations were performed to investigate the molecular organization of peptide 2. The simulations were started with the peptides initiated in several anti-parallel β-sheet orientations (supporting Information is provided herein) and followed their structural evolution through about 0.5 microseconds. We observed stable anti-parallel β-sheets formed by the peptide 2, stabilized by hydrogen bonding between the backbone, and salt-bridging between charged side-chains (lysine and glutamic acid), as well as the uncapped C-terminus between neighboring peptides. Interestingly, spontaneous assembly of peptide 2 β-sheets was observed, mediated by hydrophobic interactions of phenyl-alanine side chains and Fmoc groups. Therefore disclosed is the all-atom structure shown in FIGS. 16A and 16B with a hydrophobic core and a hydrophilic exterior formed by acidic and basic side chains.

Overall, both the experimental observations and molecular simulations showed that while the self-assembled nano-platelets formed by peptide 2 may contain some other organized or disordered structures, the β-sheet structure arrangement is a predominant and favorable one.

Enzyme instructed formation of nanoplatelets by peptide 1. After confirming the β-sheet structure arrangement of peptide 2 and its successful staining with ThT, peptide 1 was designed to detect legumain activity (see FIGS. 1A, 1B, and 1C). To solubilize peptide 2 in aqueous solutions and prevent its aggregation in the absence of legumain activity, a hydrophilic motif (GEEGSGEE) was added to peptide 2. The hydrolysis of peptide 1 by legumain was confirmed by performing liquid chromatography-mass spectrometry (LC-MS) analysis (See FIGS. 17A and 17B), which showed that almost 30% of the peptide was cleaved by legumain to form the self-assembly precursor, peptide 2.

Similar to peptide 2, the self-assembled structures of peptide 1 formed upon cleavage by legumain could be easily collected by brief centrifugation, as shown in FIG. 18. In the absence of legumain, on the other hand, no precipitate was observed (see FIG. 18, left panel). The morphology of the aggregates formed by peptide 1 after incubation with legumain was investigated using TEM and AFM. Before imaging, aggregates were bath sonicated to break up the aggregates and facilitate high resolution imaging. FIGS. 6A and 6B show TEM images of the nanoplatelets formed by peptide 1 in the presence of legumain. While the overall morphology of the aggregates formed by the cleavage product of peptide 1 was different from peptide 2 aggregates, similar nanoplatelet structures were observed in the sonicated sample (see FIG. 6B). AFM measurements further confirmed the formation of nanoplatelets with a similar thickness to the platelets observed for peptide 2 as shown in FIGS. 7A and 7B. These results indicate peptide 2 molecules generated upon hydrolysis of peptide 1 by legumain form the nanoplatelets.

CD measurements were used to show the formation of β-sheet structures by peptide 1 in the presence of legumain as shown in FIG. 8. Before addition of legumain, the CD spectrum of peptide 1 indicated a random coil organization without β-sheet formation. Upon legumain addition, the CD spectrum of peptide 1 started to change, and the two major peaks observed for peptide 2 (at about 195 nm and about 218 nm) appeared in the first 10-15 min of measurement, indicating the formation of β-sheet structures. These two peaks rapidly evolved in the first about one hour. After that, the change was slower but continued for about one day, where the CD spectrum of peptide 1 was almost identical to the CD spectrum of peptide 2 as shown in FIG. 5A. Further incubation of the peptide 1 solution up to about three days resulted in only a slight change in the spectrum. A CD spectrum of the same solution was collected after two weeks (See FIG. 19), which did not show any significant change in the spectrum and indicated long-term stability of the formed structures. FTIR measurements were also performed with peptide 1 in the presence or absence of legumain as shown in FIG. 13. In the absence of legumain, only a main random coil peak at 1643 cm⁻¹ was observed. After incubation with legumain an FTIR spectrum almost identical to that of peptide 2 was obtained with anti-parallel β-sheet peaks at 1624 and 1688 cm⁻¹, a random coil peak at 1643 cm⁻¹, and a low-intensity α-helix peak at 1660 cm⁻¹.

Development of legumain activity assay using peptide 1 and Thioflavin T. Next, peptide 1 and ThT were applied to detect the activity of legumain. When ThT (90 μM) was added to the peptide 1 solution (1.0 mg/mL), only a small enhancement (1.4 fold) in the ThT emission was observed (See FIG. 9A), indicating good solubility of peptide 1. Then, peptide 1 was incubated with different amounts of legumain (10-1000 ng/mL) for about two hours and ThT (90 μM) was added. A gradual increase in the ThT emission intensity was observed with increasing legumain concentration, reaching an enhancement in the intensity of about 32 fold at 1000 ng/mL legumain concentration as shown in FIGS. 5A and 5B. In addition, a linear response was found at low legumain concentrations between about 10 to about 200 ng/mL (See FIG. 20). While a slight (about 1.3 fold) fluorescence enhancement was obtained at the legumain concentration of 10.0 ng/mL, an easily detectable (about 3-fold) fluorescence enhancement was detected at 25.0 ng/mL. Accordingly, the limit of detection (LOD) and limit of quantification (LOQ) values were determined to be about 12 ng/mL (0.21 nM) and about 25 ng/mL (0.45 nM), respectively.

The absorbance spectra of peptide 1 incubated with different amounts of legumain were also recorded after incubating the probe with ThT as shown in FIG. 21. With increasing legumain concentrations, the absorbance spectrum of ThT steadily red-shifted of from about 413 nm to about 423 nm, suggesting the binding of ThT molecules to the self-assembled ß-sheet structures formed in response to legumain activity (Sulatskaya, A. I. et al., 2010).

The effect of peptide concentration on assay performance was also studied. Peptide 1 samples at different concentrations (0.05 mg/mL to 1.0 mg/mL) were incubated in assay buffer in the presence (500 ng/mL) or absence of legumain as shown in FIGS. 22A, and 22B. At peptide concentration below 0.25 mg/mL, the ThT emission intensity increase was minimal (1.3-1.4 fold). At a peptide concentration of about 0.25 mg/mL and above, the fluorescence intensity of the ThT was gradually increased, and a 28-fold enhancement in its emission was obtained at 1 mg/mL peptide concentration. Importantly, no significant enhancement in the ThT fluorescence was observed in the absence of legumain, even at the highest peptide concentration. It was observed that increasing the peptide concentration beyond about 1.0 mg/mL can cause enhancement in the background fluorescence; thus, 1.0 mg/mL was selected as a suitable concentration for the assay.

While ThT was typically added after incubating the probe with legumain in our assay, it was also shown that it could be added at the beginning. The addition of ThT prior to legumain also allowed for monitoring the change in its fluorescence over time as shown in FIG. 10. In the first 15 minutes, ThT fluorescence did not change significantly when peptide 1 (1.0 mg/mL) was incubated with legumain (1000 ng/mL) in the presence of ThT (90 μM). At around 15 min, the ThT fluorescence intensity started to increase sharply, which continued for about the next two hours. After this point, the increase in the intensity was slower but continued until the experiment was terminated at three hours. To investigate the effect of longer incubation times on fluorescence intensity of ThT, we collected fluorescence measurements from peptide 1 solutions incubated with 1000 ng/mL legumain at about 2 hours, 24 hours, and 72 hours as shown in FIG. 23. It was observed that at 24 hours, the fluorescence intensity was about 2.5 higher compared with the intensity at two hours. Incubation of the solution for an additional 48 hours did not significantly affect the intensity. These results were in accordance with the CD observations (See FIG. 8). Notably, while in this study an incubation time of about two hours was used, longer incubation times may improve the sensitivity of the assay.

Legumain activity detection in human plasma. Inhibition experiments using a legumain inhibitor, RR-11a were carried out to demonstrate that peptide 1 is selectively cleaved by legumain (Ekici, O. D. et al., Aza-Peptide Michael Acceptors: A New Class of Inhibitors Specific for Caspases and Other Clan CD Cysteine Proteases. J. Med. Chem. 2004, 47 (8), 1889-1892; Shen, L. et al., M2 Tumour-Associated Macrophages Contribute to Tumour Progression via Legumain Remodelling the Extracellular Matrix in Diffuse Large B Cell Lymphoma. Sci. Rep. 2016, 6 (1), 30347). The inhibitor at various concentrations was incubated with legumain (1000 ng/mL) before mixing with the peptide (1.0 mg/mL). FIG. 11 shows the percent inhibition of legumain activity at different RR-11a concentrations. A gradual increase in the percent inhibition of legumain activity was observed with increasing RR-11a concentrations, which reached 92% at the inhibitor concentration of 250 nM (14× excess of legumain). The results presented in FIGS. 10 and 11 suggested that the activity assay described here can be potentially used in inhibitor discovery studies.

To assess the possibility of using the developed self-assembling polypeptides and methods in complex biological environments, legumain detection experiments in human plasma were performed. In initial studies, a background fluorescence signal in plasma (20%) was detected due to the nonspecific interactions between ThT and plasma proteins (see FIG. 24A) (Rovnyagina, N. R. et al., Binding of Thioflavin T by Albumins: An Underestimated Role of Protein Oligomeric Heterogeneity. Int. J. Biol. Macromol. 2018, 108, 284-290). While this background signal was relatively strong, the incubation of peptide 1 in legumain (1000 ng/mL) spiked plasma still produced a detectable fluorescence enhancement (about 2.5 fold) after the addition of ThT (90 μM). To understand the origin of the background signal, the assay was performed in albumin depleted plasma. Albumin was depleted as it is the most abundant protein in plasma (35 mg/ML to 50 mg/mL) and it is well known that hydrophobic molecules such as drugs and dyes can bind to its hydrophobic domains (Wang, Y. R. et al., Rapid-Response Fluorescent Probe for the Sensitive and Selective Detection of Human Albumin in Plasma and Cell Culture Supernatants. Chem. Commun. 2016, 52 (36), 6064-6067). Indeed, depletion of albumin vastly reduced the background fluorescence to improve the ON/OFF ratio of the assay to about 10 (see FIG. 24B), which indicates that the nonspecific fluorescence enhancement of ThT in plasma mostly originated from its interaction with albumin. In some embodiments, improving the assay performance in biological solutions may be possible by using low albumin binding β-sheet intercalating dyes (Kim, D. et al., Two-Photon Absorbing Dyes with Minimal Autofluorescence in Tissue Imaging: Application to in Vivo Imaging of Amyloid-3 Plaques with a Negligible Background Signal. J. Am. Chem. Soc. 2015, 137 (21), 6781-6789).

It was also shown that background fluorescence of ThT in plasma could be largely eliminated by collecting the ThT labeled self-assembled structures by centrifugation and resuspending them in a buffer as shown in FIG. 25.

Having a better understanding of the assay's background fluorescence, further studies were performed to optimize the assay performance in plasma. To reduce the background fluorescence, the assay was run in plasma using lower ThT concentrations (see FIGS. 26A and 26B). As expected, lowering the ThT concentration to 25 μM or 10 μM significantly reduced the background fluorescence by 53% and 76%, respectively. It was found that at the ThT concentration of 25 μM, the fluorescence signal of the ThT labeled peptide aggregates was only reduced by 20% in comparison to the original ThT concentration of 90 μM that was used in the above studies. Accordingly, 25 μM was selected as a suitable ThT concentration for further studies in human plasma. In the optimized assay conditions, a fluorescence enhancement of about 20 fold was obtained in 10% plasma at the legumain concentration of 1000 ng/mL as shown in FIG. 12A. It was also found that the sensitivity of the assay was reduced when running in plasma (see FIG. 12B) with a minimum detectable concentration between 50 ng/mL to 200 ng/mL. One potential reason for the reduction in the assay sensitivity is the cleavage of the plasma proteins by legumain, which can, almost non-specifically, cleave the peptide bonds after asparagine residues (Dali, E. and Brandstetter, H., Structure and Function of Legumain in Health and Disease. Biochimie 2016, 122, 126-150). To see if the nonspecific cleavage of plasma proteins reduces the assay sensitivity, we performed legumain detection studies in buffer and 10% plasma using a commercially available quenched legumain probe (Z-AAN-AMC). A similar reduction in the assay sensitivity was observed for the Z-AAN-AMC self-assembling polypeptide (see FIGS. 27 and 28), indicating that the legumain cleavable sites on plasma proteins compete with the introduced substrates in the legumain activity assays. The presence of the other legumain substrates in the assay decreases the probe hydrolysis rate. This resulted in a decreased signal, especially at low legumain concentrations.

Molecular simulations of the Fmoc-FKFEAAN peptide. To obtain a molecular understanding of the self-assembled peptides, the peptide 2 (Fmoc-FKFEAAN, shown in FIG. 15A) was modeled as antiparallel beta-sheets, consistent with the CD data. To that end, model structures were used of antiparallel beta-sheets with similar amino acid sidechains as template structures, including IFQINS (4r0p.pdb)48 and IYKVEI (6c3f.pdb) (Saelices, L. et al., Crystal Structures of Amyloidogenic Segments of Human Transthyretin. Protein Sci. 2018, 27 (7), 1295-1303). While both the amyloid forming peptides have alternating hydrophobic and hydrophilic sidechains, the IFQINS peptide has all the hydrophobic sidechains on the same side of the fiber (cis), and the IYKVEI peptide has them alternating on either side of the fiber (trans). Dimer structures of these peptides mutated to Fmoc-FKFEAAN are shown in FIGS. 15B and 15C.

Molecular dynamics (MD) simulations of 6-mers of the peptides were performed in both the aforementioned configurations, and followed the evolution of their structures over a course of about 0.5 seconds simulation time. Even though the starting structures of the two configurations have similar backbone hydrogen bonding, we observed very different time-evolutions (see FIGS. 16A and 16B). The 6-mer in the trans orientation lost the beta-sheet structure over the course of the simulation, except for the dimer at the core of the sheet. However, the 6-mer in the cis orientation spontaneously split into two sheets of 3 peptides, and assembled into a beta-barrel type structure with a hydrophobic core of PHE sidechains, and a hydrophilic exterior of LYS, GLU & C-terminus charged residues. MD simulations details. The CHARMM forcefield was chosen for molecular dynamics (MD) simulations of the peptide since it has already been shown to successfully model self-assembly of peptides, and contains parameters for the Fmoc group developed by Tuttle & coworkers (MacKerell, A. D. et al., All-Atom Empirical Potential for Molecular Modeling and Dynamics Studies of Proteins. J. Phys. Chem. B 1998, 102 (18), 3586-3616; Brooks, B. R.; Brooks, C. L. et al., CHARMM: The Biomolecular Simulation Program. J. Comput. Chem. 2009, 30 (10), 1545-1614; Ramos Sasselli, I. et al., CHARMM Force Field Parameterization Protocol for Self-Assembling Peptide Amphiphiles: The Fmoc Moiety. Phys. Chem. Chem. Phys. 2016, 18 (6), 4659-4667). 6-mer beta-sheets of the peptides in cis and trans orientations of the side chains were studied as mentioned above. All MD simulations were performed using Gromacs-2018 package(Abraham, M. J. et al., GROMACS: High Performance Molecular Simulations through Multi-Level Parallelism from Laptops to Supercomputers. SoftwareX2015, 1-2, 19-25). The simulation system included the beta-sheet in water in a 3D periodic box. The initial box size was 5.0×5.0×5.0 nm³ containing the peptides, about 4000 water molecules, and 6 Na+ counterions for charge neutrality. The system was subjected to energy minimization to prevent any overlap of atoms, followed by a 1.0 nanosecond (ns) equilibration run. The equilibrated system was then subjected to a 0.5 microsecond (s) production run. The MD simulations incorporated leap-frog algorithm with a 2 femtosecond (fs) timestep to integrate the equations of motion. The system was maintained at 300 K and 1 bar, using the velocity rescaling thermostat and Parrinello-Rahman barostat, respectively (Bussi, G.; Donadio, D.; Parrinello, M. Canonical Sampling through Velocity Rescaling. J. Chem. Phys. 2007, 126 (1), 014101; Berendsen, H. J. C. et al., Molecular Dynamics with Coupling to an External Bath. J. Chem. Phys. 1984, 81 (8), 3684-3690). The long-ranged electrostatic interactions were calculated using particle mesh Ewald (PME) algorithm with a real space cutoff of 1.2 nm (Darden, T. et al., Particle Mesh Ewald: An N,N Log(N) Method for Ewald Sums in Large Systems. J. Chem. Phys. 1993, 98 (12), 10089-10092). LJ interactions were also truncated at 1.2 nm. TIP3P model was used represent the water molecules, and LINCS algorithm was used to constrain the motion of hydrogen atoms bonded to heavy atoms (Jorgensen, W. L. et al., Comparison of Simple Potential Functions for Simulating Liquid Water. J. Chem. Phys. 1983, 79 (2), 926-935; Hess, B.; Bekker, H. et al., G. E. M. LINCS: A Linear Constraint Solver for Molecular Simulations. J. Comput. Chem. 1997, 18 (12), 1463-1472). Coordinates of the peptide were stored every 100 picoseconds (ps) for visualization and analysis using Visual Molecular Dynamics (VMD) (Humphrey, W. et al., VMD: Visual Molecular Dynamics. J. Mol. Graph. 1996, 14 (1), 33-38.).

### Example 2—Enzyme-Instructed Formation of Beta-Sheet by Cathepsin B

To show the general applicability of the disclosed methods and ß-strand motif for the sensing of proteases, a third peptide for sensing a different protease was designed and the assay was run as before. This new peptide, peptide 3, was designed by substituting the legumain protease substrate of peptide 1 for that of a different protease, cathepsin B. Peptide 3 similarly has a β-strand forming motif and a hydrophilic motif, but the protease substrate motif was changed to LAGGAG (SEQ ID NO: 146), which is preferentially cleaved by cathepsin B between as follows: LAG/GAG. The full sequence of peptide 3 is Fmoc-FKFELAGGAGEEGSGEEE (SEQ ID NO: 208). Cathepsin B is a cysteine protease that is upregulated in various cancers, pre-cancerous lesions, and other disease states, including arthritis. FIG. 29A shows that the fluorescence intensity of ThT with peptide 3 significantly increases after cathepsin B treatment. FIG. 29B shows up to a 72 fold increase in ThT fluorescence after treatment of peptide 3 with cathepsin B.

Recombinant human cathepsin B (Bio-Techne) was activated in 25 mM MES at pH 5 for 30 min at room temperature. Peptide 3 was prepared as a 2.0 mg/mL solution in 1× phosphate buffered saline, pH 7.4 and 5% DMSO. In a typical assay experiment, 50 μL of the peptide 3 solution was mixed 50 μL of 50 mM MES buffer, pH 5 with cathepsin B at a concentration between about 0 and about 1000 ng/mL in a 96-well microplate and the plate was incubated at 37° C. for 2 hours. Then, 10 μL of 0.1 μm-filtered 1 mM aqueous ThT solution was added to each well and mixed for a final concentration of 90 μM ThT. After 15-30 min incubation at room temperature, the ThT fluorescence was measured at room temperature using a Tecan Spark 20M microplate reader.

As disclosed herein, a novel label-free protease detection method was developed using enzyme instructed formation of β-sheet rich nanoplatelets and an intercalating dye, ThT. As disclosed herein, an unlabeled peptide was designed that is highly soluble in aqueous solutions, which comprises three building blocks: i) a β-strand motif, a legumain protease substrate motif, and a hydrophilic motif. Hydrolysis of the legumain protease substrate motif by legumain initiated the self-assembly of the unlabeled peptide into nanoplatelets with an anti-parallel β-sheet structure arrangement. A ThT dye was used to detect and quantify the formed β-sheet rich structures upon enzyme instructed self-assembly. It was demonstrated that this assay could be used to detect legumain activity in buffer solutions and human plasma selectively. The method can be applied to the detection of other proteases by changing the protease substrate motif of the self-assembling polypeptide to a different amino acid recognition sequence. In some embodiments, other β-sheet intercalating dyes may be used in the assay. In some embodiments, the method disclosed herein may be used in alternative applications, from enzyme-triggered hydrogelation to in vivo imaging of protease activity.

It will be obvious to those having skill in the art that many changes may be made to the details of the above described embodiments without departing from the underlying principles of the invention. The scope of the present invention should, therefore, be determined only by the following claims.

1. A self-assembling polypeptide, comprising:
a β-strand motif configured to self-assemble with one or more nominally identical β-strand motifs and form an anti-parallel beta-sheet structure, the β-strand motif being operatively connected to a hydrophilic motif by a protease substrate motif, the protease substrate motif comprising a protease cleavage site configured to specifically hybridize with a protease, whereby, when in an aqueous milieu and upon hybridization of the protease to the protease cleavage site, the protease cleaves the self-assembling polypeptide and dissociates the β-strand motif allowing the dissociated β-strand motif to self-assemble with the one or more nominally identical β-strand motifs and thereby form the anti-parallel β-sheet structure.

2. The self-assembling polypeptide of claim 1, in which, the β-strand motif comprises, from N-terminus to C-terminus, an amino acid sequence selected from any one of:
(SEQ ID NO: 1)
Fmoc-Phe-Lys-Phe-Glu,
(SEQ ID NO: 2)
Fmoc-Phe-Phe,
(SEQ ID NO: 3)
Fmoc-Phe-Phe-(d-Lys)-(d-Lys)
(SEQ ID NO: 4)
Fmoc-Phe-(d-Lys)-Phe-(d-Lys)
(SEQ ID NO: 5)
Phe-Glu-Phe-Glu-Phe-Lys-Phe-Lys,
(SEQ ID NO: 6)
Phe-Glu-Phe-Lys-Phe-Glu-Phe-Lys,
(SEQ ID NO: 7)
Phe-Lys-Phe-Glu-Phe-Lys-Phe-Glu,
(SEQ ID NO: 8)
(d-Phe)-(d-Lys)-(d-Phe)-(d-Glu)-
(d-Phe)-(d-Lys)-(d-Phe)-(d-Glu)
(SEQ ID NO: 9)
Acetyl-Phe-Lys-Phe-Glu-Phe-Lys-Phe-
Glu-Amide,
(SEQ ID NO: 10)
and Acetyl-Phe-Lys-Phe-Glu-Phe-Lys-
Phe-Amide.

3. The self-assembling polypeptide of claim 1, in which the net charge of the hydrophilic motif is negative.

4. The self-assembling polypeptide of claim 3, in which the hydrophilic motif comprises, from N-terminus to C-terminus, an amino acid sequence selected from any one of:
(SEQ ID NO: 11)
Glu-Glu-Glu-Gly-Ser-Gly-Glu-Glu-Glu,
(SEQ ID NO: 12)
Asp-Asp-Asp-Gly-Ser-Gly-Glu-Glu-Glu,
(SEQ ID NO: 13)
Glu-Glu-Glu-Gly-Ser-Gly-Asp-Asp-Asp,
(SEQ ID NO: 14)
Glu-Glu-Glu-Gly-Ser-Gly-Glu-Glu-Glu-
Gly-Ser-Gly-Glu-Glu-Glu,
(SEQ ID NO: 15)
Asp-Asp-Asp-Gly-Ser-Gly-Glu-Glu-
Glu-Gly-Ser-Gly-Glu-Glu-Glu,
(SEQ ID NO: 16)
Glu-Glu-Glu-Gly-Ser-Gly-Asp-Asp-
Asp-Gly-Ser-Gly-Glu-Glu-Glu,
(SEQ ID NO: 17)
Asp-Asp-Asp-Gly-Ser-Gly-Asp-Asp-
Asp-Gly-Ser-Gly-Asp-Asp-Asp,
(SEQ ID NO: 18)
Asp-Asp-Gly-Asp-Asp,
(SEQ ID NO: 19)
Asp-Asp-Gly-Asp-Asp-Gly-Asp-Asp,
(SEQ ID NO: 20)
Asp-Asp-Gly-Asp-Asp-Gly-Asp-Asp-
Gly-Asp-Asp,
(SEQ ID NO: 21)
Asp-Asp-Gly-Asp-Asp-Gly-Asp-Asp-
Gly-Asp-Asp-Gly-Asp-Asp,
(SEQ ID NO: 22)
Glu-Glu-Gly-Glu-Glu,
(SEQ ID NO: 23)
Glu-Glu-Gly-Glu-Glu-Gly-Glu-Glu,
(SEQ ID NO: 24)
Glu-Glu-Gly-Glu-Glu-Gly-Glu-Glu-
Gly-Glu-Glu,
(SEQ ID NO: 25)
Glu-Glu-Gly-Glu-Glu-Gly-Glu-Glu-
Gly-Glu-Glu-Gly-Glu-Glu,
(SEQ ID NO: 26)
Glu-Glu-Gly-Lys-Asp-Asp-Gly-Glu-
Glu-Gly-Asp-Asp,
(SEQ ID NO: 27)
Asp-Asp-Gly-Glu-Glu-Gly-Asp-Asp-
Gly-Glu-Glu,
(SEQ ID NO: 28)
Glu-Glu-Gly-Lys-Lys-Gly-Glu-Glu-
Gly-Lys-Lys-Gly-Glu-Glu,
and
(SEQ ID NO: 29)
Asp-Asp-Gly-Glu-Glu-Gly-Lys-Lys-
Gly-Glu-Glu-Gly-Lys-Lys,
(SEQ ID NO: 30)
Asp-Ser-Asp-Ser,
(SEQ ID NO: 31)
Asp-Ser-Asp-Ser-Asp-Ser,
(SEQ ID NO: 32)
Asp-Ser-Asp-Ser-Asp-Ser-Asp-Ser,
(SEQ ID NO: 33)
Asp-Ser-Asp-Ser-Asp-Ser-Asp-Ser-
Asp-Ser,
(SEQ ID NO: 34)
Asp-Ser-Asp-Ser-Asp-Ser-Asp-Ser-
Asp-Ser-Asp-Ser,
(SEQ ID NO: 35)
Asp-Ser-Asp-Ser-Asp-Ser-Asp-Ser-
Asp-Ser-Asp-Ser-Asp-Ser,
(SEQ ID NO: 36)
Asp-Ser-Asp-Ser-Asp-Ser-Asp-Ser-
Asp-Ser-Asp-Ser-Asp-Ser-Asp-Ser,
(SEQ ID NO: 37)
Glu-Ser-Glu-Ser,
(SEQ ID NO: 38)
Glu-Ser-Glu-Ser-Glu-Ser,
(SEQ ID NO: 39)
Glu-Ser-Glu-Ser-Glu-Ser-Glu-Ser,
(SEQ ID NO: 40)
Glu-Ser-Glu-Ser-Glu-Ser-Glu-Ser-Glu-Ser,
(SEQ ID NO: 41)
Glu-Ser-Glu-Ser-Glu-Ser-Glu-Ser-Glu-
Ser-Glu-Ser,
(SEQ ID NO: 42)
Glu-Ser-Glu-Ser-Glu-Ser-Glu-Ser-Glu-
Ser-Glu-Ser-Glu-Ser,
(SEQ ID NO: 43)
Glu-Ser-Glu-Ser-Glu-Ser-Glu-Ser-Glu-
Ser-Glu-Ser-Glu-Ser-Glu-Ser,
(SEQ ID NO: 44)
Glu-Glu,
(SEQ ID NO: 45)
Glu-Glu-Glu,
(SEQ ID NO: 46)
Glu-Glu-Glu-Glu,
(SEQ ID NO: 47)
Glu-Glu-Glu-Glu-Glu,
(SEQ ID NO: 48)
Glu-Glu-Glu-Glu-Glu-Glu,
(SEQ ID NO: 49)
Glu-Glu-Glu-Glu-Glu-Glu-Glu,
(SEQ ID NO: 50)
Glu-Glu-Glu-Glu-Glu-Glu-Glu-Glu,
(SEQ ID NO: 51)
Glu-Glu-Glu-Glu-Glu-Glu-Glu-Glu-Glu,
(SEQ ID NO: 52)
Glu-Glu-Glu-Glu-Glu-Glu-Glu-Glu-Glu-Glu,
(SEQ ID NO: 53)
Asp-Asp,
(SEQ ID NO: 54)
Asp-Asp-Asp,
(SEQ ID NO: 55)
Asp-Asp-Asp-Asp,
(SEQ ID NO: 56)
Asp-Asp-Asp-Asp-Asp,
(SEQ ID NO: 57)
Asp-Asp-Asp-Asp-Asp-Asp,
(SEQ ID NO: 58)
Asp-Asp-Asp-Asp-Asp-Asp-Asp,
(SEQ ID NO: 59)
Asp-Asp-Asp-Asp-Asp-Asp-Asp-Asp,
(SEQ ID NO: 60)
Asp-Asp-Asp-Asp-Asp-Asp-Asp-Asp-Asp,
(SEQ ID NO: 61)
Asp-Asp-Asp-Asp-Asp-Asp-Asp-Asp-Asp-Asp,
(SEQ ID NO: 62)
Glu-Asp,
(SEQ ID NO: 63)
Glu-Asp-Glu-Asp,
(SEQ ID NO: 64)
Glu-Asp-Glu-Asp-Glu-Asp,
(SEQ ID NO: 65)
Glu-Asp-Glu-Asp-Glu-Asp-Glu-Asp,
(SEQ ID NO: 66)
Glu-Asp-Glu-Asp-Glu-Asp-Glu-Asp-Glu-Asp,
(SEQ ID NO: 67)
Asp-Glu,
(SEQ ID NO: 68)
Asp-Glu-Asp-Glu,
(SEQ ID NO: 69)
Asp-Glu-Asp-Glu-Asp-Glu,
(SEQ ID NO: 70)
Asp-Glu-Asp-Glu-Asp-Glu-Asp-Glu,
(SEQ ID NO: 71)
Asp-Glu-Asp-Glu-Asp-Glu-Asp-Glu-Asp-Glu,
(SEQ ID NO: 72)
and pSer-pSer-Gly-Ser-Gly-pSer-pSer.

5. The self-assembling polypeptide of claim 1, in which the hydrophilic motif comprises a zwitterion.

6. The self-assembling polypeptide of claim 5, in which the hydrophilic motif comprises, from N-terminus to C-terminus, an amino acid sequence selected from any one of: Lys-Glu-Lys-Glu-Lys (SEQ ID NO: 73), Lys-Glu-Lys-Glu-Lys-Glu-Lys (SEQ ID NO: 74), Lys-Glu-Lys-Glu-Lys-Glu-Lys-Glu-Lys (SEQ ID NO: 75), Lys-Glu-Lys-Glu-Lys-Glu-Lys-Glu-Lys-Glu-Lys (SEQ ID NO: 76), Arg-Asp-Arg-Asp-Arg (SEQ ID NO: 77), Arg-Asp-Arg-Asp-Arg-Asp-Arg (SEQ ID NO: 78), Arg-Asp-Arg-Asp-Arg-Asp-Arg-Asp-Arg (SEQ ID NO: 79), Arg-Asp-Arg-Asp-Asp-Arg-Asp-Arg-Asp-Arg-Arg (SEQ ID NO: 80), Arg-Glu-Arg-Glu-Arg (SEQ ID NO: 81), Arg-Glu-Arg-Glu-Arg-Glu-Arg (SEQ ID NO: 82), Arg-Glu-Arg-Glu-Arg-Glu-Arg-Glu-Arg (SEQ ID NO: 83), Arg-Glu-Arg-Glu-Arg-Glu-Arg-Glu-Arg-Glu-Arg (SEQ ID NO: 84), Lys-Asp-Lys-Asp-Lys (SEQ ID NO: 85), Lys-Asp-Lys-Asp-Lys-Asp-Lys (SEQ ID NO: 86), Lys-Asp-Lys-Asp-Lys-Asp-Lys (SEQ ID NO: 87), Lys-Asp-Lys-Asp-Lys-Asp-Lys (SEQ ID NO: 88), pSer-Lys-pSer-Lys-Lys (SEQ ID NO: 89), pSer-Lys-pSer-Lys-pSer-Lys-Lys (SEQ ID NO: 90), pSer-Lys-pSer-Lys-pSer-Lys-pSer-Lys-Lys (SEQ ID NO: 91), pSer-Lys-pSer-Lys-pSer-Lys-pSer-Lys-pSer-Lys-Lys (SEQ ID NO: 92), pSer-Arg-pSer-Arg-Arg (SEQ ID NO: 93), pSer-Arg-pSer-Arg-pSer-Arg-Arg (SEQ ID NO: 94), pSer-Arg-pSer-Arg-pSer-Arg-pSer-Arg-Arg (SEQ ID NO: 95), pSer-Arg-pSer-Arg-pSer-Arg-pSer-Arg-pSer-Arg-Arg (SEQ ID NO: 96), Ser-Lys-Asp-Ser-Lys-Asp-Lys (SEQ ID NO: 97), Ser-Lys-Asp-Ser-Lys-Asp-Ser-Lys-Asp-Lys (SEQ ID NO: 98), Ser-Lys-Asp-Ser-Lys-Asp-Ser-Lys-Asp-Ser-Lys-Asp-Lys (SEQ ID NO: 99), Ser-Arg-Glu-Ser-Arg-Glu-Arg (SEQ ID NO: 100), Ser-Arg-Glu-Ser-Arg-Glu-Ser-Arg-Glu-Arg (SEQ ID NO: 101), Ser-Arg-Glu-Ser-Arg-Glu-Ser-Arg-Glu-Ser-Arg-Glu-Arg (SEQ ID NO: 102), Ser-Lys-Glu-Ser-Lys-Glu-Lys (SEQ ID NO: 103), Ser-Lys-Glu-Ser-Lys-Glu-Ser-Lys-Glu-Lys (SEQ ID NO: 104), Ser-Lys-Glu-Ser-Lys-Glu-Ser-Lys-Glu-Ser-Lys-Glu-Lys (SEQ ID NO: 105), Ser-Arg-Asp-Ser-Arg-Asp-Arg (SEQ ID NO: 106), Ser-Arg-Asp-Ser-Arg-Asp-Ser-Arg-Asp-Arg (SEQ ID NO: 107), and Ser-Arg-Asp-Ser-Arg-Asp-Ser-Arg-Asp-Ser-Arg-Asp-Arg (SEQ ID NO: 108).

7. The self-assembling polypeptide of claim 5, in which the hydrophilic motif comprises, from N-terminus to C-terminus, an amino acid sequence selected from any one of: Lys-Glu-Lys-Glu (SEQ ID NO: 109), Lys-Glu-Lys-Glu-Lys-Glu (SEQ ID NO: 110), Lys-Glu-Lys-Glu-Lys-Glu-Lys-Glu (SEQ ID NO: 111), Lys-Glu-Lys-Glu-Lys-Glu-Lys-Glu-Lys-Glu (SEQ ID NO: 112), Arg-Asp-Arg-Asp (SEQ ID NO: 113), Arg-Asp-Arg-Asp-Arg-Asp (SEQ ID NO: 114), Arg-Asp-Arg-Asp-Arg-Asp-Arg-Asp (SEQ ID NO: 115), Arg-Asp-Arg-Asp-Asp-Arg-Asp-Arg-Asp-Arg (SEQ ID NO: 116), Arg-Glu-Arg-Glu (SEQ ID NO: 117), Arg-Glu-Arg-Glu-Arg-Glu (SEQ ID NO: 118), Arg-Glu-Arg-Glu-Arg-Glu-Arg-Glu (SEQ ID NO: 119), Arg-Glu-Arg-Glu-Arg-Glu-Arg-Glu-Arg-Glu (SEQ ID NO: 120), Lys-Asp-Lys-Asp (SEQ ID NO: 121), Lys-Asp-Lys-Asp-Lys-Asp (SEQ ID NO: 122), Lys-Asp-Lys-Asp-Lys-Asp (SEQ ID NO: 123), Lys-Asp-Lys-Asp-Lys-Asp (SEQ ID NO: 124), pSer-Lys-pSer-Lys (SEQ ID NO: 125), pSer-Lys-pSer-Lys-pSer-Lys (SEQ ID NO: 126), pSer-Lys-pSer-Lys-pSer-Lys-pSer-Lys (SEQ ID NO: 127), pSer-Lys-pSer-Lys-pSer-Lys-pSer-Lys-pSer-Lys (SEQ ID NO: 128), pSer-Arg-pSer-Arg (SEQ ID NO: 129), pSer-Arg-pSer-Arg-pSer-Arg (SEQ ID NO: 130), pSer-Arg-pSer-Arg-pSer-Arg-pSer-Arg (SEQ ID NO: 131), pSer-Arg-pSer-Arg-pSer-Arg-pSer-Arg-pSer-Arg (SEQ ID NO: 132), Ser-Lys-Asp-Ser-Lys-Asp (SEQ ID NO: 133), Ser-Lys-Asp-Ser-Lys-Asp-Ser-Lys-Asp (SEQ ID NO: 134), Ser-Lys-Asp-Ser-Lys-Asp-Ser-Lys-Asp-Ser-Lys-Asp (SEQ ID NO: 135), Ser-Arg-Glu-Ser-Arg-Glu (SEQ ID NO: 136), Ser-Arg-Glu-Ser-Arg-Glu-Ser-Arg-Glu (SEQ ID NO: 137), Ser-Arg-Glu-Ser-Arg-Glu-Ser-Arg-Glu-Ser-Arg-Glu (SEQ ID NO: 138), Ser-Lys-Glu-Ser-Lys-Glu (SEQ ID NO: 139), Ser-Lys-Glu-Ser-Lys-Glu-Ser-Lys-Glu (SEQ ID NO: 140), Ser-Lys-Glu-Ser-Lys-Glu-Ser-Lys-Glu-Ser-Lys-Glu (SEQ ID NO: 141), Ser-Arg-Asp-Ser-Arg-Asp (SEQ ID NO: 142), Ser-Arg-Asp-Ser-Arg-Asp-Ser-Arg-Asp (SEQ ID NO: 143), and Ser-Arg-Asp-Ser-Arg-Asp-Ser-Arg-Asp-Ser-Arg-Asp (SEQ ID NO: 144), in which the C-terminus is amidated.

8. The self-assembling polypeptide of claim 1, in which the protease substrate motif comprises an amino acid sequence selected from any one of: Ala-Ala-Asn-Gly (SEQ ID NO: 145), Leu-Ala-Gly-Gly-Ala-Gly (SEQ ID NO: 146), Arg-Ser-Lys-Arg-Val-Ser-Gly (SEQ ID NO: 147), Arg-Ser-Lys-Arg-Ser (SEQ ID NO: 148), Ser-Ala-Gln-Ala-Val-Val-Ser-Gln (SEQ ID NO: 149), Ala-Gln-Ala-Val-Val-Ser (SEQ ID NO: 150), Leu-Ala-Gln-Ala-Val-Val-Ser-Ala (SEQ ID NO: 151), Ala-Gln-Ala-Val-Val-Ser (SEQ ID NO: 152), Leu-Ala-Ala-Ala-Val-Val-Ser-Ser (SEQ ID NO: 153), Ala-Ala-Ala-Val-Val (SEQ ID NO: 154), Pro-Ala-Ala-Ala-Gln-Arg-Leu-Arg (SEQ ID NO: 155), Ala-Ala-Ala-Gln-Arg-Leu (SEQ ID NO: 156), Leu-Pro-Ala-Ala-Leu-Val-Gly-Ala (SEQ ID NO: 157), Pro-Ala-Ala-Leu (SEQ ID NO: 158), Leu-Pro-Ser-Gly-Leu-Val-Gly-Ala (SEQ ID NO: 159), Pro-Ser-Gly-Leu (SEQ ID NO: 160), Gly-Pro-Ala-Gly-Leu-Ala-Gly-Ala (SEQ ID NO: 161), Pro-Ala-Gly-Leu (SEQ ID NO: 162), Gly-Pro-Gly-Gly-Leu-Ala-Gly-Ala (SEQ ID NO: 163), Gly-Pro-Leu-Gly-Leu-Val-Gly-Gln (SEQ ID NO: 164), Pro-Leu-Gly-Leu (SEQ ID NO: 165), Gly-Pro-Ala-Gly-Leu-Gly-Gly-Gly (SEQ ID NO: 166), Pro-Ala-Gly-Leu (SEQ ID NO: 167), Gly-Pro-Pro-Gly-Leu-Arg-Gly-Pro (SEQ ID NO: 168), Pro-Pro-Gly-Leu (SEQ ID NO: 169), Gly-Pro-Leu-Gly-Leu-Arg-Gly-Pro (SEQ ID NO: 170), Pro-Leu-Gly-Leu (SEQ ID NO: 171), Gly-Pro-Ala-Gly-Leu-Arg-Thr-Glu (SEQ ID NO: 172), Leu-Pro-Gln-Gly-Leu-Ala-Gly-Arg (SEQ ID NO: 173), Pro-Ala-Gly-Leu (SEQ ID NO: 174), Glu-Ala-Glu-Asn-Gly-Glu-Leu-Pro (SEQ ID NO: 175), Ala-Ala-Asn-Gly (SEQ ID NO: 176), Asp-Asn-Phe-Leu-Val (SEQ ID NO: 177), Asp-Asn-Phe-Phe-Val (SEQ ID NO: 178), Gly-Leu-Ala-Gly-Gly-Ala-Gly-Gly (SEQ ID NO: 179), Leu-Ala-Gly-Gly-Ala-Gly (SEQ ID NO: 180), Gly-Leu-Val-Ala-Leu-Leu-Ala-Gly-Gly (SEQ ID NO: 181), Leu-Glu-Val-Leu-Ile-Val (SEQ ID NO: 182), Glu-Val-Leu-Ile-Val (SEQ ID NO: 183), Glu-Val-Val-Leu-Val-Ala-Leu-Ala (SEQ ID NO: 184), Glu-Val-Val-Phe-Val-Ala-Leu-Ala (SEQ ID NO: 185), Val-Leu-Val-Ala (SEQ ID NO: 186), Val-Phe-Val-Ala (SEQ ID NO: 187), Asp-Val-Leu-Leu-Ser-Trp-Ala-Val (SEQ ID NO: 188), Val-Leu-Leu-Ser-Trp (SEQ ID NO: 189), Ala-Lys-Leu-Lys-Glu-Glu-Asp-Asp (SEQ ID NO: 190), Ala-Gly-Leu-Gly-Glu-Glu-Asp-Asp (SEQ ID NO: 191), Ala-Leu-Leu-Gly-Ala-Pro-Pro-Pro (SEQ ID NO: 192), Gly-Leu-Leu-Gly-Ser-Glu-Pro-Glu (SEQ ID NO: 193), Leu-Gly-Ala-Pro (SEQ ID NO: 194), Leu-Gly-Ser-Glu (SEQ ID NO: 195), Ala-Ala-Lys-Gly-Ala-Ala-Pro-Glu (SEQ ID NO: 196), Leu-Gly-Ala-Ala (SEQ ID NO: 197), Ser-Ser-Gln-Tyr-Ser-Ser-Asn-Gly (SEQ ID NO: 198), Ser-Gln-Gln-Tyr-Ser-Ser-Asn-Gly (SEQ ID NO: 199), Ser-Ser-Gln-Gln-Ser-Ser-Asn-Gly (SEQ ID NO: 200), Gly-Gly-Ser-Arg-Ser-Gly-Gly-Gly (SEQ ID NO: 201), Gly-Gly-Ser-Arg-Ser-Pro-Gly-Gly (SEQ ID NO: 202), Gly-Val-Asn-Leu-Asp-Val-Glu-Val (SEQ ID NO: 203), Arg-Gln-Ala-Arg-Lys-Val-Gly-Gly (SEQ ID NO: 204), and Ala-Ala-Ala-Arg-Lys-Val-Gly-Gly (SEQ ID NO: 205).

9. The self-assembling polypeptide of any of the preceding cl claim 1, in which the self-assembling polypeptide is utilized as means for detecting a protease in an aqueous milieu by the protease triggering the enzyme-instructed self-assembly of the self-assembling polypeptide to form the anti-parallel β-sheet structure, the aqueous milieu comprising a β-sheet intercalating dye that emits fluorescent light upon intercalating with the anti-parallel β-sheet structure.

10. The self-assembling polypeptide of claim 9, in which detecting the protease is utilized as means for detecting a cancer selected from any of: a liver cancer, a skin cancer, a cervical cancer, a brain cancer, a lung cancer, a stomach cancer, a bile-duct cancer, a gastric cancer, a pancreatic cancer, a bladder cancer, a breast cancer, a lung cancer, a prostate cancer, a kidney cancer, a brain cancer, a colorectal cancer, an ovarian cancer, a thyroid cancer, a melanoma cancer, and a thyroid cancer.

11. The self-assembling polypeptide of claim 9, in which detecting the protease is utilized as means for detecting a disease, disorder, or syndrome selected from any of: a pre-cancerous breast hyperplasia, a prostate disorder, an Alzheimer's disease, an acute coronary disease, an atherosclerosis disease, an arthritis disease, an osteoarthritis disease, an osteoporosis disease, and an acute coronary syndrome.

12. A method for detecting proteolytic cleavage by enzyme-instructed β-sheet formation, the method comprising:
administering, into an aqueous milieu, a set of one or more self-assembling polypeptides of claim 1;
administering, into the aqueous milieu, a β-sheet intercalating dye configured to emit a fluorescent signal upon forming a complex with one or more anti-parallel β-sheet structures formed by the self-assembly of β-strand motifs dissociated from their respective self-assembling polypeptides by proteolytic cleavage and thereby indicate the presence of the protease in the aqueous milieu; and
detecting the fluorescent signal.

13. The method of claim 12, wherein the β-sheet intercalating dye is selected from a MCAAD-3 dye, a ThT dye, a Thioflavin T dye, a Thioflavin S dye, and a Congo Red dye.

14. The method of claim 12, in which the method is used to detect a cancer selected from any of: a liver cancer, a skin cancer, a cervical cancer, a brain cancer, a lung cancer, a stomach cancer, a bile-duct cancer, a gastric cancer, a pancreatic cancer, a bladder cancer, a breast cancer, a lung cancer, a prostate cancer, a kidney cancer, a brain cancer, a colorectal cancer, an ovarian cancer, a thyroid cancer, a melanoma cancer, and a thyroid cancer.

15. The method of claim 12, in which the method is used to detect a disease, disorder, or syndrome selected from any of: a pre-cancerous breast hyperplasia, a prostate disorder, an Alzheimer's disease, an acute coronary disease, an atherosclerosis disease, an arthritis disease, an osteoarthritis disease, an osteoporosis disease, and an acute coronary syndrome.

16. The method of claim 12, in which the aqueous milieu is a plasma sample obtained from a subject.

17. A kit, comprising:
a set of one or more self-assembling polypeptide of claim 1; and
a β-sheet intercalating dye.

18. The kit of claim 17, in which the β-sheet intercalating dye is selected from a MCAAD-3 dye, a ThT dye, a Thioflavin T dye, a Thioflavin S dye, and a Congo Red dye.

19. The kit of claim 17, in which the kit is used to detect a cancer selected from any of: a liver cancer, a skin cancer, a cervical cancer, a brain cancer, a lung cancer, a stomach cancer, a bile-duct cancer, a gastric cancer, a pancreatic cancer, a bladder cancer, a breast cancer, a lung cancer, a prostate cancer, a kidney cancer, a brain cancer, a colorectal cancer, an ovarian cancer, a thyroid cancer, a melanoma cancer, and a thyroid cancer.

20. The kit of claim 17, in which the kit is used to detect a disease, disorder, or syndrome selected from any of: a pre-cancerous breast hyperplasia, a prostate disorder, an Alzheimer's disease, an acute coronary disease, an atherosclerosis disease, an arthritis disease, an osteoarthritis disease, an osteoporosis disease, and an acute coronary syndrome.


SEQUENCE LISTING (208 sequences)

SEQ ID NO: 1 - protein, 4 aa, synthetic construct
  REGION 1..4: note = SEQ ID NO: 1 is an amino acid sequence of an exemplary -strandmotif,consisting of the amino acid sequence:Fmoc-Phe-Lys-Phe-Glu, in which the N-terminus is modified tocomprise a Fmoc protecting group.
FKFE

SEQ ID NO: 2 - skipped

SEQ ID NO: 3 - protein, 4 aa, synthetic construct
  REGION 1..4: note = SEQ ID NO: 3 is an amino acid sequence of an exemplary -strandmotif consisting of the amino acid sequence:Fmoc-Phe-Phe-(D-Lys)-(D-Lys), in which the N-terminus is modifiedto comprise a Fmoc protecting group.
FFKK

SEQ ID NO: 4 - protein, 4 aa, synthetic construct
  REGION 1..4: note = SEQ ID NO: 4 is an amino acid sequence of an exemplary -strandmotif consisting of the amino acid sequence:Fmoc-Phe-(D-Lys)-Phe-(D-Lys),in which the N-terminus is modifiedto comprise a Fmoc protecting group.
FKFK

SEQ ID NO: 5 - protein, 8 aa, synthetic construct
  REGION 1..8: note = SEQ ID NO: 5 is an amino acid sequence of an exemplary -strandmotif consisting of the amino acid sequence:Phe-Glu-Phe-Glu-Phe-Lys-Phe-Lys.
FEFEFKFK

SEQ ID NO: 6 - protein, 8 aa, synthetic construct
  REGION 1..8: note = SEQ ID NO: 6 is an amino acid sequence of an exemplary -strandmotif consisting of the amino acid sequence:Phe-Glu-Phe-Lys-Phe-Glu-Phe-Lys.
FEFKFEFK

SEQ ID NO: 7 - protein, 8 aa, synthetic construct
  REGION 1..8: note = SEQ ID NO: 7 is an amino acid sequence of an exemplary -strandmotif consisting of the amino acid sequence:Phe-Lys-Phe-Glu-Phe-Lys-Phe-Glu.
FKFEFKFE

SEQ ID NO: 8 - protein, 8 aa, synthetic construct
  REGION 1..8: note = SEQ ID NO: 8 is an amino acid sequence of an exemplary -strandmotif consisting of the amino acid sequence:(D-Phe)-(D-Lys)-(D-Phe)-(D-Glu)-(D-Phe)-(D-Lys)-( D-Phe)-(D-Glu).
FKFEFKFE

SEQ ID NO: 9 - protein, 8 aa, synthetic construct
  REGION 1..8: note = SEQ ID NO: 9 is an amino acid sequence of an exemplary -strandmotif consisting of the amino acid sequence:Acetyl-Phe-Lys-Phe-Glu-Phe-Lys-Phe-Glu-Amide, in which theC-terminus and N-terminus are modified to comprise, respectively,
FKFEFKFE

SEQ ID NO: 10 - protein, 7 aa, synthetic construct
  REGION 1..7: note = SEQ ID NO: 10 is an amino acid sequence of an exemplary -strandmotif consisting of the amino acid sequence:Acetyl-Phe-Lys-Phe-Glu-Phe-Lys-Phe-Amide.
FKFEFKF

SEQ ID NO: 11 - protein, 9 aa, synthetic construct
  REGION 1..9: note = SEQ ID NO: 11 is an amino acid sequence of an exemplaryhydrophilic motif consisting of the amino acid sequence:Glu-Glu-Glu-Gly-Ser-Gly-Glu-Glu-Glu.
EEEGSGEEE

SEQ ID NO: 12 - protein, 9 aa, synthetic construct
  REGION 1..9: note = SEQ ID NO: 12 is an amino acid sequence of an exemplaryhydrophilic motif consisting of the amino acid sequence:Asp-Asp-Asp-Gly-Ser-Gly-Glu-Glu-Glu.
DDDGSGEEE

SEQ ID NO: 13 - protein, 9 aa, synthetic construct
  REGION 1..9: note = SEQ ID NO: 13 is an amino acid sequence of an exemplaryhydrophilic motif consisting of the amino acid sequence:Glu-Glu-Glu-Gly-Ser-Gly-Asp-Asp-Asp.
EEEGSGDDD

SEQ ID NO: 14 - protein, 15 aa, synthetic construct
  REGION 1..15: note = SEQ ID NO: 14 is an amino acid sequence of an exemplaryhydrophilic motif consisting of the amino acid sequence:Glu-Glu-Glu-Gly-Ser-Gly-Glu-Glu-Glu-Gly-Ser-Gly-G lu-Glu-Glu.
EEEGSGEEEGSGEEE

SEQ ID NO: 15 - protein, 15 aa, synthetic construct
  REGION 1..15: note = SEQ ID NO: 15 is an amino acid sequence of an exemplaryhydrophilic motif consisting of the amino acid sequence:Asp-Asp-Asp-Gly-Ser-Gly-Glu-Glu-Glu-Gly-Ser-Gly-G lu-Glu-Glu.
DDDGSGEEEGSGEEE

SEQ ID NO: 16 - protein, 15 aa, synthetic construct
  REGION 1..15: note = SEQ ID NO: 16 is an amino acid sequence of an exemplaryhydrophilic motif consisting of the amino acid sequence:Glu-Glu-Glu-Gly-Ser-Gly-Asp-Asp-Asp-Gly-Ser-Gly-G lu-Glu-Glu.
EEEGSGDDDGSGEEE

SEQ ID NO: 17 - protein, 15 aa, synthetic construct
  REGION 1..15: note = SEQ ID NO: 17 is an amino acid sequence of an exemplaryhydrophilic motif consisting of the amino acid sequence:Asp-Asp-Asp-Gly-Ser-Gly-Asp-Asp-Asp-Gly-Ser-Gly-A sp-Asp-Asp.
DDDGSGDDDGSGDDD

SEQ ID NO: 18 - protein, 5 aa, synthetic construct
  REGION 1..5: note = SEQ ID NO: 18 is an amino acid sequence of an exemplaryhydrophilic motif consisting of the amino acid sequence:Asp-Asp-Gly-Asp-Asp.
DDGDD

SEQ ID NO: 19 - protein, 8 aa, synthetic construct
  REGION 1..8: note = SEQ ID NO: 19 is an amino acid sequence of an exemplaryhydrophilic motif consisting of the amino acid sequence:Asp-Asp-Gly-Asp-Asp-Gly-Asp-Asp.
DDGDDGDD

SEQ ID NO: 20 - protein, 11 aa, synthetic construct
  REGION 1..11: note = SEQ ID NO: 20 is an amino acid sequence of an exemplaryhydrophilic motif consisting of the amino acid sequence:Asp-Asp-Gly-Asp-Asp-Gly-Asp-Asp-Gly-Asp-Asp.
DDGDDGDDGDD

SEQ ID NO: 21 - protein, 14 aa, synthetic construct
  REGION 1..14: note = SEQ ID NO: 21 is an amino acid sequence of an exemplaryhydrophilic motif consisting of the amino acid sequence:Asp-Asp-Gly-Asp-Asp-Gly-Asp-Asp-Gly-Asp-Asp-Gly-A sp-Asp.
DDGDDGDDGDDGDD

SEQ ID NO: 22 - protein, 5 aa, synthetic construct
  REGION 1..5: note = SEQ ID NO: 22 is an amino acid sequence of an exemplaryhydrophilic motif consisting of the amino acid sequence:Glu-Glu-Gly-Glu-Glu.
EEGEE

SEQ ID NO: 23 - protein, 8 aa, synthetic construct
  REGION 1..8: note = SEQ ID NO: 23 is an amino acid sequence of an exemplaryhydrophilic motif consisting of the amino acid sequence:Glu-Glu-Gly-Glu-Glu-Gly-Glu-Glu.
EEGEEGEE

SEQ ID NO: 24 - protein, 11 aa, synthetic construct
  REGION 1..11: note = SEQ ID NO: 24 is an amino acid sequence of an exemplaryhydrophilic motif consisting of the amino acid sequence:Glu-Glu-Gly-Glu-Glu-Gly-Glu-Glu-Gly-Glu-Glu.
EEGEEGEEGEE

SEQ ID NO: 25 - protein, 14 aa, synthetic construct
  REGION 1..14: note = SEQ ID NO: 25 is an amino acid sequence of an exemplaryhydrophilic motif consisting of the amino acid sequence:Glu-Glu-Gly-Glu-Glu-Gly-Glu-Glu-Gly-Glu-Glu-Gly-G lu-Glu.
EEGEEGEEGEEGEE

SEQ ID NO: 26 - protein, 12 aa, synthetic construct
  REGION 1..12: note = SEQ ID NO: 26 is an amino acid sequence of an exemplaryhydrophilic motif consisting of the amino acid sequence:Glu-Glu-Gly-Lys-Asp-Asp-Gly-Glu-Glu-Gly-Asp-Asp.
EEGKDDGEEGDD

SEQ ID NO: 27 - protein, 11 aa, synthetic construct
  REGION 1..11: note = SEQ ID NO: 27 is an amino acid sequence of an exemplaryhydrophilic motif consisting of the amino acid sequence:Asp-Asp-Gly-Glu-Glu-Gly-Asp-Asp-Gly-Glu-Glu.
DDGEEGDDGEE

SEQ ID NO: 28 - protein, 14 aa, synthetic construct
  REGION 1..14: note = SEQ ID NO: 28 is an amino acid sequence of an exemplaryhydrophilic motif consisting of the amino acid sequence:Glu-Glu-Gly-Lys-Lys-Gly-Glu-Glu-Gly-Lys-Lys-Gly-G lu-Glu.
EEGKKGEEGKKGEE

SEQ ID NO: 29 - protein, 14 aa, synthetic construct
  REGION 1..14: note = SEQ ID NO: 29 is an amino acid sequence of an exemplaryhydrophilic motif consisting of the amino acid sequence:Asp-Asp-Gly-Glu-Glu-Gly-Lys-Lys-Gly-Glu-Glu-Gly-L ys-Lys.
DDGEEGKKGEEGKK

SEQ ID NO: 30 - protein, 4 aa, synthetic construct
  REGION 1..4: note = SEQ ID NO: 30 is an amino acid sequence of an exemplaryhydrophilic motif consisting of the amino acid sequence:Asp-Ser-Asp-Ser.
DSDS

SEQ ID NO: 31 - protein, 6 aa, synthetic construct
  REGION 1..6: note = SEQ ID NO: 31 is an amino acid sequence of an exemplaryhydrophilic motif consisting of the amino acid sequence:Asp-Ser-Asp-Ser-Asp-Ser.
DSDSDS

SEQ ID NO: 32 - protein, 8 aa, synthetic construct
  REGION 1..8: note = SEQ ID NO: 32 is an amino acid sequence of an exemplaryhydrophilic motif consisting of the amino acid sequence:Asp-Ser-Asp-Ser-Asp-Ser-Asp-Ser.
DSDSDSDS

SEQ ID NO: 33 - protein, 10 aa, synthetic construct
  REGION 1..10: note = SEQ ID NO: 33 is an amino acid sequence of an exemplaryhydrophilic motif consisting of the amino acid sequence:Asp-Ser-Asp-Ser-Asp-Ser-Asp-Ser-Asp-Ser.
DSDSDSDSDS

SEQ ID NO: 34 - protein, 12 aa, synthetic construct
  REGION 1..12: note = SEQ ID NO: 34 is an amino acid sequence of an exemplaryhydrophilic motif consisting of the amino acid sequence:Asp-Ser-Asp-Ser-Asp-Ser-Asp-Ser-Asp-Ser-Asp-Ser.
DSDSDSDSDSDS

SEQ ID NO: 35 - protein, 14 aa, synthetic construct
  REGION 1..14: note = SEQ ID NO: 35 is an amino acid sequence of an exemplaryhydrophilic motif consisting of the amino acid sequence:Asp-Ser-Asp-Ser-Asp-Ser-Asp-Ser-Asp-Ser-Asp-Ser-A sp-Ser.
DSDSDSDSDSDSDS

SEQ ID NO: 36 - protein, 16 aa, synthetic construct
  REGION 1..16: note = SEQ ID NO: 36 is an amino acid sequence of an exemplaryhydrophilic motif consisting of the amino acid sequence:Asp-Ser-Asp-Ser-Asp-Ser-Asp-Ser-Asp-Ser-Asp-Ser-A sp-Ser-Asp-Ser.
DSDSDSDSDSDSDSDS

SEQ ID NO: 37 - protein, 4 aa, synthetic construct
  REGION 1..4: note = SEQ ID NO: 37 is an amino acid sequence of an exemplaryhydrophilic motif consisting of the amino acid sequence:Glu-Ser-Glu-Ser.
ESES

SEQ ID NO: 38 - protein, 6 aa, synthetic construct
  REGION 1..6: note = SEQ ID NO: 38 is an amino acid sequence of an exemplaryhydrophilic motif consisting of the amino acid sequence:Glu-Ser-Glu-Ser-Glu-Ser.
ESESES

SEQ ID NO: 39 - protein, 8 aa, synthetic construct
  REGION 1..8: note = SEQ ID NO: 39 is an amino acid sequence of an exemplaryhydrophilic motif consisting of the amino acid sequence:Glu-Ser-Glu-Ser-Glu-Ser-Glu-Ser.
ESESESES

SEQ ID NO: 40 - protein, 10 aa, synthetic construct
  REGION 1..10: note = SEQ ID NO: 40 is an amino acid sequence of an exemplaryhydrophilic motif consisting of the amino acid sequence:Glu-Ser-Glu-Ser-Glu-Ser-Glu-Ser-Glu-Ser.
ESESESESES

SEQ ID NO: 41 - protein, 12 aa, synthetic construct
  REGION 1..12: note = SEQ ID NO: 41 is an amino acid sequence of an exemplaryhydrophilic motif consisting of the amino acid sequence:Glu-Ser-Glu-Ser-Glu-Ser-Glu-Ser-Glu-Ser-Glu-Ser.
ESESESESESES

SEQ ID NO: 42 - protein, 14 aa, synthetic construct
  REGION 1..14: note = SEQ ID NO: 42 is an amino acid sequence of an exemplaryhydrophilic motif consisting of the amino acid sequence:Glu-Ser-Glu-Ser-Glu-Ser-Glu-Ser-Glu-Ser-Glu-Ser-G lu-Ser.
ESESESESESESES

SEQ ID NO: 43 - protein, 16 aa, synthetic construct
  REGION 1..16: note = SEQ ID NO: 43 is an amino acid sequence of an exemplaryhydrophilic motif consisting of the amino acid sequence:Glu-Ser-Glu-Ser-Glu-Ser-Glu-Ser-Glu-Ser-Glu-Ser-G lu-Ser-Glu-Ser.
ESESESESESESESES

SEQ ID NO: 44-45 - skipped (2 entries)

SEQ ID NO: 46 - protein, 4 aa, synthetic construct
  REGION 1..4: note = SEQ ID NO: 46 is an amino acid sequence of an exemplaryhydrophilic motif consisting of the amino acid sequence:Glu-Glu-Glu-Glu.
EEEE

SEQ ID NO: 47 - protein, 5 aa, synthetic construct
  REGION 1..5: note = SEQ ID NO: 47 is an amino acid sequence of an exemplaryhydrophilic motif consisting of the amino acid sequence:Glu-Glu-Glu-Glu-Glu.
EEEEE

SEQ ID NO: 48 - protein, 6 aa, synthetic construct
  REGION 1..6: note = SEQ ID NO: 48 is an amino acid sequence of an exemplaryhydrophilic motif consisting of the amino acid sequence:Glu-Glu-Glu-Glu-Glu-Glu.
EEEEEE

SEQ ID NO: 49 - protein, 7 aa, synthetic construct
  REGION 1..7: note = SEQ ID NO: 49 is an amino acid sequence of an exemplaryhydrophilic motif consisting of the amino acid sequence:Glu-Glu-Glu-Glu-Glu-Glu-Glu.
EEEEEEE

SEQ ID NO: 50 - protein, 8 aa, synthetic construct
  REGION 1..8: note = SEQ ID NO: 50 is an amino acid sequence of an exemplaryhydrophilic motif consisting of the amino acid sequence:Glu-Glu-Glu-Glu-Glu-Glu-Glu-Glu.
EEEEEEEE

SEQ ID NO: 51 - protein, 9 aa, synthetic construct
  REGION 1..9: note = SEQ ID NO: 51 is an amino acid sequence of an exemplaryhydrophilic motif consisting of the amino acid sequence:Glu-Glu-Glu-Glu-Glu-Glu-Glu-Glu-Glu.
EEEEEEEEE

SEQ ID NO: 52 - protein, 10 aa, synthetic construct
  REGION 1..10: note = SEQ ID NO: 52 is an amino acid sequence of an exemplaryhydrophilic motif consisting of the amino acid sequence:Glu-Glu-Glu-Glu-Glu-Glu-Glu-Glu-Glu-Glu.
EEEEEEEEEE

SEQ ID NO: 53-54 - skipped (2 entries)

SEQ ID NO: 55 - protein, 4 aa, synthetic construct
  REGION 1..4: note = SEQ ID NO: 55 is an amino acid sequence of an exemplaryhydrophilic motif consisting of the amino acid sequence:Asp-Asp-Asp-Asp.
DDDD

SEQ ID NO: 56 - protein, 5 aa, synthetic construct
  REGION 1..5: note = SEQ ID NO: 56 is an amino acid sequence of an exemplaryhydrophilic motif consisting of the amino acid sequence:Asp-Asp-Asp-Asp-Asp.
DDDDD

SEQ ID NO: 57 - protein, 6 aa, synthetic construct
  REGION 1..6: note = SEQ ID NO: 57 is an amino acid sequence of an exemplaryhydrophilic motif consisting of the amino acid sequence:Asp-Asp-Asp-Asp-Asp-Asp.
DDDDDD

SEQ ID NO: 58 - protein, 7 aa, synthetic construct
  REGION 1..7: note = SEQ ID NO: 58 is an amino acid sequence of an exemplaryhydrophilic motif consisting of the amino acid sequence:Asp-Asp-Asp-Asp-Asp-Asp-Asp.
DDDDDDD

SEQ ID NO: 59 - protein, 8 aa, synthetic construct
  REGION 1..8: note = SEQ ID NO: 59 is an amino acid sequence of an exemplaryhydrophilic motif consisting of the amino acid sequence:Asp-Asp-Asp-Asp-Asp-Asp-Asp-Asp.
DDDDDDDD

SEQ ID NO: 60 - protein, 9 aa, synthetic construct
  REGION 1..9: note = SEQ ID NO: 60 is an amino acid sequence of an exemplaryhydrophilic motif consisting of the amino acid sequence:Asp-Asp-Asp-Asp-Asp-Asp-Asp-Asp-Asp.
DDDDDDDDD

SEQ ID NO: 61 - protein, 10 aa, synthetic construct
  REGION 1..10: note = SEQ ID NO: 61 is an amino acid sequence of an exemplaryhydrophilic motif consisting of the amino acid sequence:Asp-Asp-Asp-Asp-Asp-Asp-Asp-Asp-Asp-Asp.
DDDDDDDDDD

SEQ ID NO: 62 - skipped

SEQ ID NO: 63 - protein, 4 aa, synthetic construct
  REGION 1..4: note = SEQ ID NO: 63 is an amino acid sequence of an exemplaryhydrophilic motif consisting of the amino acid sequence:Glu-Asp-Glu-Asp.
EDED

SEQ ID NO: 64 - protein, 6 aa, synthetic construct
  REGION 1..6: note = SEQ ID NO: 64 is an amino acid sequence of an exemplaryhydrophilic motif consisting of the amino acid sequence:Glu-Asp-Glu-Asp-Glu-Asp.
EDEDED

SEQ ID NO: 65 - protein, 8 aa, synthetic construct
  REGION 1..8: note = SEQ ID NO: 65 is an amino acid sequence of an exemplaryhydrophilic motif consisting of the amino acid sequence:Glu-Asp-Glu-Asp-Glu-Asp-Glu-Asp.
EDEDEDED

SEQ ID NO: 66 - protein, 10 aa, synthetic construct
  REGION 1..10: note = SEQ ID NO: 66 is an amino acid sequence of an exemplaryhydrophilic motif consisting of the amino acid sequence:Glu-Asp-Glu-Asp-Glu-Asp-Glu-Asp-Glu-Asp.
EDEDEDEDED

SEQ ID NO: 67 - skipped

SEQ ID NO: 68 - protein, 4 aa, synthetic construct
  REGION 1..4: note = SEQ ID NO: 68 is an amino acid sequence of an exemplaryhydrophilic motif consisting of the amino acid sequence:Asp-Glu-Asp-Glu.
DEDE

SEQ ID NO: 69 - protein, 6 aa, synthetic construct
  REGION 1..6: note = SEQ ID NO: 69 is an amino acid sequence of an exemplaryhydrophilic motif consisting of the amino acid sequence:Asp-Glu-Asp-Glu-Asp-Glu.
DEDEDE

SEQ ID NO: 70 - protein, 8 aa, synthetic construct
  REGION 1..8: note = SEQ ID NO: 70 is an amino acid sequence of an exemplaryhydrophilic motif consisting of the amino acid sequence:Asp-Glu-Asp-Glu-Asp-Glu-Asp-Glu.
DEDEDEDE

SEQ ID NO: 71 - protein, 10 aa, synthetic construct
  REGION 1..10: note = SEQ ID NO: 71 is an amino acid sequence of an exemplaryhydrophilic motif consisting of the amino acid sequence:Asp-Glu-Asp-Glu-Asp-Glu-Asp-Glu-Asp-Glu.
DEDEDEDEDE

SEQ ID NO: 72 - protein, 7 aa, synthetic construct
  REGION 1..7: note = SEQ ID NO: 72 is an amino acid sequence of an exemplaryhydrophilic motif consisting of the amino acid sequence:pSer-pSer-Gly-Ser-Gly-pSer-pSer.
SSGSGSS

SEQ ID NO: 73 - protein, 5 aa, synthetic construct
  REGION 1..5: note = SEQ ID NO: 73 is an amino acid sequence of an exemplaryhydrophilic motif consisting of the amino acid sequence:Lys-Glu-Lys-Glu-Lys
KEKEK

SEQ ID NO: 74 - protein, 7 aa, synthetic construct
  REGION 1..7: note = SEQ ID NO: 74 is an amino acid sequence of an exemplaryhydrophilic motif consisting of the amino acid sequence:Lys-Glu-Lys-Glu-Lys-Glu-Lys.
KEKEKEK

SEQ ID NO: 75 - protein, 9 aa, synthetic construct
  REGION 1..9: note = SEQ ID NO: 75 is an amino acid sequence of an exemplaryhydrophilic motif consisting of the amino acid sequence:Lys-Glu-Lys-Glu-Lys-Glu-Lys-Glu-Lys.
KEKEKEKEK

SEQ ID NO: 76 - protein, 11 aa, synthetic construct
  REGION 1..11: note = SEQ ID NO: 76 is an amino acid sequence of an exemplaryhydrophilic motif consisting of the amino acid sequence:Lys-Glu-Lys-Glu-Lys-Glu-Lys-Glu-Lys-Glu-Lys.
KEKEKEKEKEK

SEQ ID NO: 77 - protein, 5 aa, synthetic construct
  REGION 1..5: note = SEQ ID NO: 77 is an amino acid sequence of an exemplaryhydrophilic motif consisting of the amino acid sequence:Arg-Asp-Arg-Asp-Arg.
RDRDR

SEQ ID NO: 78 - protein, 7 aa, synthetic construct
  REGION 1..7: note = SEQ ID NO: 78 is an amino acid sequence of an exemplaryhydrophilic motif consisting of the amino acid sequence:Arg-Asp-Arg-Asp-Arg-Asp-Arg.
RDRDRDR

SEQ ID NO: 79 - protein, 9 aa, synthetic construct
  REGION 1..9: note = SEQ ID NO: 79 is an amino acid sequence of an exemplaryhydrophilic motif consisting of the amino acid sequence:Arg-Asp-Arg-Asp-Arg-Asp-Arg-Asp-Arg.
RDRDRDRDR

SEQ ID NO: 80 - protein, 11 aa, synthetic construct
  REGION 1..11: note = SEQ ID NO: 80 is an amino acid sequence of an exemplaryhydrophilic motif consisting of the amino acid sequence:Arg-Asp-Arg-Asp-Asp-Arg-Asp-Arg-Asp-Arg-Arg.
RDRDDRDRDRR

SEQ ID NO: 81 - protein, 5 aa, synthetic construct
  REGION 1..5: note = SEQ ID NO: 81 is an amino acid sequence of an exemplaryhydrophilic motif consisting of the amino acid sequence:Arg-Glu-Arg-Glu-Arg.
RERER

SEQ ID NO: 82 - protein, 7 aa, synthetic construct
  REGION 1..7: note = SEQ ID NO: 82 is an amino acid sequence of an exemplaryhydrophilic motif consisting of the amino acid sequence:Arg-Glu-Arg-Glu-Arg-Glu-Arg.
RERERER

SEQ ID NO: 83 - protein, 9 aa, synthetic construct
  REGION 1..9: note = SEQ ID NO: 83 is an amino acid sequence of an exemplaryhydrophilic motif consisting of the amino acid sequence:Arg-Glu-Arg-Glu-Arg-Glu-Arg-Glu-Arg.
RERERERER

SEQ ID NO: 84 - protein, 11 aa, synthetic construct
  REGION 1..11: note = SEQ ID NO: 84 is an amino acid sequence of an exemplaryhydrophilic motif consisting of the amino acid sequence:Arg-Glu-Arg-Glu-Arg-Glu-Arg-Glu-Arg-Glu-Arg.
RERERERERER

SEQ ID NO: 85 - protein, 5 aa, synthetic construct
  REGION 1..5: note = SEQ ID NO: 85 is an amino acid sequence of an exemplaryhydrophilic motif consisting of the amino acid sequence:Lys-Asp-Lys-Asp-Lys.
KDKDK

SEQ ID NO: 86 - protein, 7 aa, synthetic construct
  REGION 1..7: note = SEQ ID NO: 86 is an amino acid sequence of an exemplaryhydrophilic motif consisting of the amino acid sequence:Lys-Asp-Lys-Asp-Lys-Asp-Lys.
KDKDKDK

SEQ ID NO: 87 - protein, 7 aa, synthetic construct
  REGION 1..7: note = SEQ ID NO: 87 is an amino acid sequence of an exemplaryhydrophilic motif consisting of the amino acid sequence:Lys-Asp-Lys-Asp-Lys-Asp-Lys.
KDKDKDK

SEQ ID NO: 88 - protein, 7 aa, synthetic construct
  REGION 1..7: note = SEQ ID NO: 88 is an amino acid sequence of an exemplaryhydrophilic motif consisting of the amino acid sequence:Lys-Asp-Lys-Asp-Lys-Asp-Lys.
KDKDKDK

SEQ ID NO: 89 - protein, 5 aa, synthetic construct
  REGION 1..5: note = SEQ ID NO: 89 is an amino acid sequence of an exemplaryhydrophilic motif consisting of the amino acid sequence:pSer-Lys-pSer-Lys-Lys.
SKSKK

SEQ ID NO: 90 - protein, 7 aa, synthetic construct
  REGION 1..7: note = SEQ ID NO: 90 is an amino acid sequence of an exemplaryhydrophilic motif consisting of the amino acid sequence:pSer-Lys-pSer-Lys-pSer-Lys-Lys.
SKSKSKK

SEQ ID NO: 91 - protein, 9 aa, synthetic construct
  REGION 1..9: note = SEQ ID NO: 91 is an amino acid sequence of an exemplaryhydrophilic motif consisting of the amino acid sequence:pSer-Lys-pSer-Lys-pSer-Lys-pSer-Lys-Lys.
SKSKSKSKK

SEQ ID NO: 92 - protein, 11 aa, synthetic construct
  REGION 1..11: note = SEQ ID NO: 92 is an amino acid sequence of an exemplaryhydrophilic motif consisting of the amino acid sequence:pSer-Lys-pSer-Lys-pSer-Lys-pSer-Lys-pSer-Lys-Lys.
SKSKSKSKSKK

SEQ ID NO: 93 - protein, 5 aa, synthetic construct
  REGION 1..5: note = SEQ ID NO: 93 is an amino acid sequence of an exemplaryhydrophilic motif consisting of the amino acid sequence:pSer-Arg-pSer-Arg-Arg.
SRSRR

SEQ ID NO: 94 - protein, 7 aa, synthetic construct
  REGION 1..7: note = SEQ ID NO: 94 is an amino acid sequence of an exemplaryhydrophilic motif consisting of the amino acid sequence:pSer-Arg-pSer-Arg-pSer-Arg-Arg.
SRSRSRR

SEQ ID NO: 95 - protein, 9 aa, synthetic construct
  REGION 1..9: note = SEQ ID NO: 95 is an amino acid sequence of an exemplaryhydrophilic motif consisting of the amino acid sequence:pSer-Arg-pSer-Arg-pSer-Arg-pSer-Arg-Arg.
SRSRSRSRR

SEQ ID NO: 96 - protein, 11 aa, synthetic construct
  REGION 1..11: note = SEQ ID NO: 96 is an amino acid sequence of an exemplaryhydrophilic motif consisting of the amino acid sequence:pSer-Arg-pSer-Arg-pSer-Arg-pSer-Arg-pSer-Arg-Arg.
SRSRSRSRSRR

SEQ ID NO: 97 - protein, 7 aa, synthetic construct
  REGION 1..7: note = SEQ ID NO: 97 is an amino acid sequence of an exemplaryhydrophilic motif consisting of the amino acid sequence:Ser-Lys-Asp-Ser-Lys-Asp-Lys.
SKDSKDK

SEQ ID NO: 98 - protein, 10 aa, synthetic construct
  REGION 1..10: note = SEQ ID NO: 98 is an amino acid sequence of an exemplaryhydrophilic motif consisting of the amino acid sequence:Ser-Lys-Asp-Ser-Lys-Asp-Ser-Lys-Asp-Lys.
SKDSKDSKDK

SEQ ID NO: 99 - protein, 13 aa, synthetic construct
  REGION 1..13: note = SEQ ID NO: 99 is an amino acid sequence of an exemplaryhydrophilic motif consisting of the amino acid sequence:Ser-Lys-Asp-Ser-Lys-Asp-Ser-Lys-Asp-Ser-Lys-Asp-L ys.
SKDSKDSKDSKDK

SEQ ID NO: 100 - protein, 7 aa, synthetic construct
  REGION 1..7: note = SEQ ID NO: 100 is an amino acid sequence of an exemplaryhydrophilic motif consisting of the amino acid sequence:Ser-Arg-Glu-Ser-Arg-Glu-Arg.
SRESRER

SEQ ID NO: 101 - protein, 10 aa, synthetic construct
  REGION 1..10: note = SEQ ID NO: 101 is an amino acid sequence of an exemplaryhydrophilic motif consisting of the amino acid sequence:Ser-Arg-Glu-Ser-Arg-Glu-Ser-Arg-Glu-Arg.
SRESRESRER

SEQ ID NO: 102 - protein, 13 aa, synthetic construct
  REGION 1..13: note = SEQ ID NO: 102 is an amino acid sequence of an exemplaryhydrophilic motif consisting of the amino acid sequence:Ser-Arg-Glu-Ser-Arg-Glu- Ser-Arg-Glu-Ser-Arg-Glu-Arg.
SRESRESRESRER

SEQ ID NO: 103 - protein, 7 aa, synthetic construct
  REGION 1..7: note = SEQ ID NO: 103 is an amino acid sequence of an exemplaryhydrophilic motif consisting of the amino acid sequence:Ser-Lys-Glu-Ser-Lys-Glu-Lys.
SKESKEK

SEQ ID NO: 104 - protein, 10 aa, synthetic construct
  REGION 1..10: note = SEQ ID NO: 104 is an amino acid sequence of an exemplaryhydrophilic motif consisting of the amino acid sequence:Ser-Lys-Glu-Ser-Lys-Glu-Ser-Lys-Glu-Lys.
SKESKESKEK

SEQ ID NO: 105 - protein, 13 aa, synthetic construct
  REGION 1..13: note = SEQ ID NO: 105 is an amino acid sequence of an exemplaryhydrophilic motif consisting of the amino acid sequence:Ser-Lys-Glu-Ser-Lys-Glu-Ser-Lys-Glu-Ser-Lys-Glu-L ys.
SKESKESKESKEK

SEQ ID NO: 106 - protein, 7 aa, synthetic construct
  REGION 1..7: note = SEQ ID NO: 106 is an amino acid sequence of an exemplaryhydrophilic motif consisting of the amino acid sequence:Ser-Arg-Asp-Ser-Arg-Asp-Arg.
SRDSRDR

SEQ ID NO: 107 - protein, 10 aa, synthetic construct
  REGION 1..10: note = SEQ ID NO: 107 is an amino acid sequence of an exemplaryhydrophilic motif consisting of the amino acid sequence:Ser-Arg-Asp-Ser-Arg-Asp-Ser-Arg-Asp-Arg.
SRDSRDSRDR

SEQ ID NO: 108 - protein, 13 aa, synthetic construct
  REGION 1..13: note = SEQ ID NO: 108 is an amino acid sequence of an exemplaryhydrophilic motif consisting of the amino acid sequence:Ser-Arg-Asp-Ser-Arg-Asp-Ser-Arg-Asp-Ser-Arg-Asp-A rg.
SRDSRDSRDSRDR

SEQ ID NO: 109 - protein, 4 aa, synthetic construct
  REGION 1..4: note = SEQ ID NO: 109 is an amino acid sequence of an exemplaryhydrophilic motif consisting of the amino acid sequence:Lys-Glu-Lys-Glu, in which the C-terminus of the amino acidsequence is amidated.
KEKE

SEQ ID NO: 110 - protein, 6 aa, synthetic construct
  REGION 1..6: note = SEQ ID NO: 110 is an amino acid sequence of an exemplaryhydrophilic motif consisting of the amino acid sequence:Lys-Glu-Lys-Glu-Lys-Glu, in which the C-terminus of the aminoacid sequence is amidated.
KEKEKE

SEQ ID NO: 111 - protein, 8 aa, synthetic construct
  REGION 1..8: note = SEQ ID NO: 111 is an amino acid sequence of an exemplaryhydrophilic motif consisting of the amino acid sequence:Lys-Glu-Lys-Glu-Lys-Glu-Lys-Glu, in which the C-terminus of theamino acid sequence is amidated.
KEKEKEKE

SEQ ID NO: 112 - protein, 10 aa, synthetic construct
  REGION 1..10: note = SEQ ID NO: 112 is an amino acid sequence of an exemplaryhydrophilic motif consisting of the amino acid sequence:Lys-Glu-Lys-Glu-Lys-Glu-Lys-Glu-Lys-Glu, in which the C-terminusof the amino acid sequence is amidated.
KEKEKEKEKE

SEQ ID NO: 113 - protein, 4 aa, synthetic construct
  REGION 1..4: note = SEQ ID NO: 113 is an amino acid sequence of an exemplaryhydrophilic motif consisting of the amino acid sequence:Arg-Asp-Arg-Asp, in which the C-terminus of the amino acidsequence is amidated.
RDRD

SEQ ID NO: 114 - protein, 6 aa, synthetic construct
  REGION 1..6: note = SEQ ID NO: 114 is an amino acid sequence of an exemplaryhydrophilic motif consisting of the amino acid sequence:Arg-Asp-Arg-Asp-Arg-Asp, in which the C-terminus of the aminoacid sequence is amidated.
RDRDRD

SEQ ID NO: 115 - protein, 8 aa, synthetic construct
  REGION 1..8: note = SEQ ID NO: 115 is an amino acid sequence of an exemplaryhydrophilic motif consisting of the amino acid sequence:Arg-Asp-Arg-Asp-Arg-Asp-Arg-Asp, in which the C-terminus of theamino acid sequence is amidated.
RDRDRDRD

SEQ ID NO: 116 - protein, 10 aa, synthetic construct
  REGION 1..10: note = SEQ ID NO: 116 is an amino acid sequence of an exemplaryhydrophilic motif consisting of the amino acid sequence:Arg-Asp-Arg-Asp-Asp-Arg-Asp-Arg-Asp-Arg, in which the C-terminusof the amino acid sequence is amidated.
RDRDDRDRDR

SEQ ID NO: 117 - protein, 4 aa, synthetic construct
  REGION 1..4: note = SEQ ID NO: 117 is an amino acid sequence of an exemplaryhydrophilic motif consisting of the amino acid sequence:Arg-Glu-Arg-Glu, in which the C-terminus of the amino acidsequence is amidated.
RERE

SEQ ID NO: 118 - protein, 6 aa, synthetic construct
  REGION 1..6: note = SEQ ID NO: 118 is an amino acid sequence of an exemplaryhydrophilic motif consisting of the amino acid sequence:Arg-Glu-Arg-Glu-Arg-Glu, in which the C-terminus of the aminoacid sequence is amidated.
RERERE

SEQ ID NO: 119 - protein, 8 aa, synthetic construct
  REGION 1..8: note = SEQ ID NO: 119 is an amino acid sequence of an exemplaryhydrophilic motif consisting of the amino acid sequence:Arg-Glu-Arg-Glu-Arg-Glu-Arg-Glu, in which the C-terminus of theamino acid sequence is amidated.
RERERERE

SEQ ID NO: 120 - protein, 10 aa, synthetic construct
  REGION 1..10: note = SEQ ID NO: 120 is an amino acid sequence of an exemplaryhydrophilic motif consisting of the amino acid sequence:Arg-Glu-Arg-Glu-Arg-Glu-Arg-Glu-Arg-Glu, in which the C-terminusof the amino acid sequence is amidated.
RERERERERE

SEQ ID NO: 121 - protein, 4 aa, synthetic construct
  REGION 1..4: note = SEQ ID NO: 121 is an amino acid sequence of an exemplaryhydrophilic motif consisting of the amino acid sequence:Lys-Asp-Lys-Asp, in which the C-terminus of the amino acidsequence is amidated.
KDKD

SEQ ID NO: 122 - protein, 6 aa, synthetic construct
  REGION 1..6: note = SEQ ID NO: 122 is an amino acid sequence of an exemplaryhydrophilic motif consisting of the amino acid sequence:Lys-Asp-Lys-Asp-Lys-Asp, in which the C-terminus of the aminoacid sequence is amidated.
KDKDKD

SEQ ID NO: 123 - protein, 6 aa, synthetic construct
  REGION 1..6: note = SEQ ID NO: 123 is an amino acid sequence of an exemplaryhydrophilic motif consisting of the amino acid sequence:Lys-Asp-Lys-Asp-Lys-Asp, in which the C-terminus of the aminoacid sequence is amidated.
KDKDKD

SEQ ID NO: 124 - protein, 6 aa, synthetic construct
  REGION 1..6: note = SEQ ID NO: 124 is an amino acid sequence of an exemplaryhydrophilic motif consisting of the amino acid sequence:Lys-Asp-Lys-Asp-Lys-Asp, in which the C-terminus of the aminoacid sequence is amidated.
KDKDKD

SEQ ID NO: 125 - protein, 4 aa, synthetic construct
  REGION 1..4: note = SEQ ID NO: 125 is an amino acid sequence of an exemplaryhydrophilic motif consisting of the amino acid sequence:pSer-Lys-pSer-Lys, in which the C-terminus of the amino acidsequence is amidated.
SKSK

SEQ ID NO: 126 - protein, 6 aa, synthetic construct
  REGION 1..6: note = SEQ ID NO: 126 is an amino acid sequence of an exemplaryhydrophilic motif consisting of the amino acid sequence:pSer-Lys-pSer-Lys-pSer-Lys, in which the C-terminus of the aminoacid sequence is amidated.
SKSKSK

SEQ ID NO: 127 - protein, 8 aa, synthetic construct
  REGION 1..8: note = SEQ ID NO: 127 is an amino acid sequence of an exemplaryhydrophilic motif consisting of the amino acid sequence:pSer-Lys-pSer-Lys-pSer-Lys-pSer-Lys, in which the C-terminus ofthe amino acid sequence is amidated.
SKSKSKSK

SEQ ID NO: 128 - protein, 10 aa, synthetic construct
  REGION 1..10: note = SEQ ID NO: 128 is an amino acid sequence of an exemplaryhydrophilic motif consisting of the amino acid sequence:pSer-Lys-pSer-Lys-pSer-Lys-pSer-Lys-pSer-Lys, in which theC-terminus of the amino acid sequence is amidated.
SKSKSKSKSK

SEQ ID NO: 129 - protein, 4 aa, synthetic construct
  REGION 1..4: note = SEQ ID NO: 129 is an amino acid sequence of an exemplaryhydrophilic motif consisting of the amino acid sequence:pSer-Arg-pSer-Arg, in which the C-terminus of the amino acidsequence is amidated.
SRSR

SEQ ID NO: 130 - protein, 4 aa, synthetic construct
  REGION 1..4: note = SEQ ID NO: 130 is an amino acid sequence of an exemplaryhydrophilic motif consisting of the amino acid sequence:pSer-Arg-pSer-Arg, in which the C-terminus of the amino acidsequence is amidated.
SRSR

SEQ ID NO: 131 - protein, 8 aa, synthetic construct
  REGION 1..8: note = SEQ ID NO: 131 is an amino acid sequence of an exemplaryhydrophilic motif consisting of the amino acid sequence:pSer-Arg-pSer-Arg-pSer-Arg-pSer-Arg, in which the C-terminus ofthe amino acid sequence is amidated.
SRSRSRSR

SEQ ID NO: 132 - protein, 9 aa, synthetic construct
  REGION 1..9: note = SEQ ID NO: 132 is an amino acid sequence of an exemplaryhydrophilic motif consisting of the amino acid sequence:pSer-Arg-pSer-Arg-pSer-Arg-pSer-Arg-pSer-Arg, in which theC-terminus of the amino acid sequence is amidated.
SRSRSRSRS

SEQ ID NO: 133 - protein, 6 aa, synthetic construct
  REGION 1..6: note = SEQ ID NO: 133 is an amino acid sequence of an exemplaryhydrophilic motif consisting of the amino acid sequence:Ser-Lys-Asp-Ser-Lys-Asp, in which the C-terminus of the aminoacid sequence is amidated.
SKDSKD

SEQ ID NO: 134 - protein, 9 aa, synthetic construct
  REGION 1..9: note = SEQ ID NO: 134 is an amino acid sequence of an exemplaryhydrophilic motif consisting of the amino acid sequence:Ser-Lys-Asp-Ser-Lys-Asp-Ser-Lys-Asp, in which the C-terminus ofthe amino acid sequence is amidated.
SKDSKDSKD

SEQ ID NO: 135 - protein, 12 aa, synthetic construct
  REGION 1..12: note = SEQ ID NO: 135 is an amino acid sequence of an exemplaryhydrophilic motif consisting of the amino acid sequence:Ser-Lys-Asp-Ser-Lys-Asp-Ser-Lys-Asp-Ser-Lys-Asp, in which theC-terminus of the amino acid sequence is amidated.
SKDSKDSKDSKD

SEQ ID NO: 136 - protein, 6 aa, synthetic construct
  REGION 1..6: note = SEQ ID NO: 136 is an amino acid sequence of an exemplaryhydrophilic motif consisting of the amino acid sequence:Ser-Arg-Glu-Ser-Arg-Glu, in which the C-terminus of the aminoacid sequence is amidated.
SRESRE

SEQ ID NO: 137 - protein, 9 aa, synthetic construct
  REGION 1..9: note = SEQ ID NO: 137 is an amino acid sequence of an exemplaryhydrophilic motif consisting of the amino acid sequence:Ser-Arg-Glu-Ser-Arg-Glu-Ser-Arg-Glu, in which the C-terminus ofthe amino acid sequence is amidated.
SRESRESRE

SEQ ID NO: 138 - protein, 12 aa, synthetic construct
  REGION 1..12: note = SEQ ID NO: 138 is an amino acid sequence of an exemplaryhydrophilic motif consisting of the amino acid sequence:Ser-Arg-Glu-Ser-Arg-Glu-Ser-Arg-Glu-Ser-Arg-Glu, in which theC-terminus of the amino acid sequence is amidated.
SRESRESRESRE

SEQ ID NO: 139 - protein, 6 aa, synthetic construct
  REGION 1..6: note = SEQ ID NO: 139 is an amino acid sequence of an exemplaryhydrophilic motif consisting of the amino acid sequence:Ser-Lys-Glu-Ser-Lys-Glu, in which the C-terminus of the aminoacid sequence is amidated.
SKESKE

SEQ ID NO: 140 - protein, 9 aa, synthetic construct
  REGION 1..9: note = SEQ ID NO: 140 is an amino acid sequence of an exemplaryhydrophilic motif consisting of the amino acid sequence:Ser-Lys-Glu-Ser-Lys-Glu-Ser-Lys-Glu, in which the C-terminus ofthe amino acid sequence is amidated.
SKESKESKE

SEQ ID NO: 141 - protein, 12 aa, synthetic construct
  REGION 1..12: note = SEQ ID NO: 141 is an amino acid sequence of an exemplaryhydrophilic motif consisting of the amino acid sequence:Ser-Lys-Glu-Ser-Lys-Glu-Ser-Lys-Glu-Ser-Lys-Glu, in which theC-terminus of the amino acid sequence is amidated.
SKESKESKESKE

SEQ ID NO: 142 - protein, 6 aa, synthetic construct
  REGION 1..6: note = SEQ ID NO: 142 is an amino acid sequence of an exemplaryhydrophilic motif consisting of the amino acid sequence:Ser-Arg-Asp-Ser-Arg-Asp, in which the C-terminus of the aminoacid sequence is amidated.
SRDSRD

SEQ ID NO: 143 - protein, 9 aa, synthetic construct
  REGION 1..9: note = SEQ ID NO: 143 is an amino acid sequence of an exemplaryhydrophilic motif consisting of the amino acid sequence:Ser-Arg-Asp-Ser-Arg-Asp-Ser-Arg-Asp, in which the C-terminus ofthe amino acid sequence is amidated.
SRDSRDSRD

SEQ ID NO: 144 - protein, 12 aa, synthetic construct
  REGION 1..12: note = SEQ ID NO: 144 is an amino acid sequence of an exemplaryhydrophilic motif consisting of the amino acid sequence:Ser-Arg-Asp-Ser-Arg-Asp-Ser-Arg-Asp-Ser-Arg-Asp, in which theC-terminus of the amino acid sequence is amidated.
SRDSRDSRDSRD

SEQ ID NO: 145 - protein, 4 aa, synthetic construct
  REGION 1..4: note = SEQ ID NO: 145 is an amino acid sequence of an exemplary proteasesubstrate motif consisting of the amino acid sequence:Ala-Ala-Asn-Gly, which comprises a protease recognition site oflegumain.
AANG

SEQ ID NO: 146 - protein, 6 aa, synthetic construct
  REGION 1..6: note = SEQ ID NO: 146 is an amino acid sequence of an exemplary proteasesubstrate motif consisting of the amino acid sequence:Leu-Ala-Gly-Gly-Ala-Gly, which comprises a protease recognitionsite of cathepsin B.
LAGGAG

SEQ ID NO: 147 - protein, 7 aa, synthetic construct
  REGION 1..7: note = SEQ ID NO: 147 is an amino acid sequence of an exemplary proteasesubstrate motif consisting of the amino acid sequence:Arg-Ser-Lys-Arg-Val-Ser-Gly, which comprises a proteaserecognition site of a furin protease.
RSKRVSG

SEQ ID NO: 148 - protein, 5 aa, synthetic construct
  REGION 1..5: note = SEQ ID NO: 148 is an amino acid sequence of an exemplary proteasesubstrate motif consisting of the amino acid sequence:Arg-Ser-Lys-Arg-Ser, which comprises a protease recognition siteof a furin protease.
RSKRS

SEQ ID NO: 149 - protein, 8 aa, synthetic construct
  REGION 1..8: note = SEQ ID NO: 149 is an amino acid sequence of an exemplary proteasesubstrate motif consisting of the amino acid sequence:Ser-Ala-Gln-Ala-Val-Val-Ser-Gln, which comprises a proteaserecognition site of an ADAM10 protease.
SAQAVVSQ

SEQ ID NO: 150 - protein, 6 aa, synthetic construct
  REGION 1..6: note = SEQ ID NO: 150 is an amino acid sequence of an exemplary proteasesubstrate motif consisting of the amino acid sequence:Ala-Gln-Ala-Val-Val-Ser, which comprises a protease recognitionsite of an ADAM10 protease.
AQAVVS

SEQ ID NO: 151 - protein, 8 aa, synthetic construct
  REGION 1..8: note = SEQ ID NO: 151 is an amino acid sequence of an exemplary proteasesubstrate motif consisting of the amino acid sequence:Leu-Ala-Gln-Ala-Val-Val-Ser-Ala, which comprises a proteaserecognition site of a TACE protease.
LAQAVVSA

SEQ ID NO: 152 - protein, 6 aa, synthetic construct
  REGION 1..6: note = SEQ ID NO: 152 is an amino acid sequence of an exemplary proteasesubstrate motif consisting of the amino acid sequence:Ala-Gln-Ala-Val-Val-Ser, which comprises a protease recognitionsite of a TACE protease.
AQAVVS

SEQ ID NO: 153 - protein, 8 aa, synthetic construct
  REGION 1..8: note = SEQ ID NO: 153 is an amino acid sequence of an exemplary proteasesubstrate motif consisting of the amino acid sequence:Leu-Ala-Ala-Ala-Val-Val-Ser-Ser, which comprises a proteaserecognition site of a TACE protease.
LAAAVVSS

SEQ ID NO: 154 - protein, 5 aa, synthetic construct
  REGION 1..5: note = SEQ ID NO: 154 is an amino acid sequence of an exemplary proteasesubstrate motif consisting of the amino acid sequence:Ala-Ala-Ala-Val-Val, which comprises a protease recognition siteof a TACE protease.
AAAVV

SEQ ID NO: 155 - protein, 8 aa, synthetic construct
  REGION 1..8: note = SEQ ID NO: 155 is an amino acid sequence of an exemplary proteasesubstrate motif consisting of the amino acid sequence:Pro-Ala-Ala-Ala-Gln-Arg-Leu-Arg, which comprises a proteaserecognition site of an ADAM8 protease.
PAAAQRLR

SEQ ID NO: 156 - protein, 6 aa, synthetic construct
  REGION 1..6: note = SEQ ID NO: 156 is an amino acid sequence of an exemplary proteasesubstrate motif consisting of the amino acid sequence:Ala-Ala-Ala-Gln-Arg-Leu, which comprises a protease recognitionsite of an ADAM8 protease.
AAAQRL

SEQ ID NO: 157 - protein, 8 aa, synthetic construct
  REGION 1..8: note = SEQ ID NO: 157 is an amino acid sequence of an exemplary proteasesubstrate motif consisting of the amino acid sequence:Leu-Pro-Ala-Ala-Leu-Val-Gly-Ala, which comprises a proteaserecognition site of a MMP-2 protease.
LPAALVGA

SEQ ID NO: 158 - protein, 4 aa, synthetic construct
  REGION 1..4: note = SEQ ID NO: 158 is an amino acid sequence of an exemplary proteasesubstrate motif consisting of the amino acid sequence:Pro-Ala-Ala-Leu, which comprises a protease recognition site of aMMP-2 protease.
PAAL

SEQ ID NO: 159 - protein, 8 aa, synthetic construct
  REGION 1..8: note = SEQ ID NO: 159 is an amino acid sequence of an exemplary proteasesubstrate motif consisting of the amino acid sequence:Leu-Pro-Ser-Gly-Leu-Val-Gly-Ala, which comprises a proteaserecognition site of a MMP-2 protease.
LPSGLVGA

SEQ ID NO: 160 - protein, 4 aa, synthetic construct
  REGION 1..4: note = SEQ ID NO: 160 is an amino acid sequence of an exemplary proteasesubstrate motif consisting of the amino acid sequence:Pro-Ser-Gly-Leu, which comprises a protease recognition site of aMMP-2 protease.
PSGL

SEQ ID NO: 161 - protein, 8 aa, synthetic construct
  REGION 1..8: note = SEQ ID NO: 161 is an amino acid sequence of an exemplary proteasesubstrate motif consisting of the amino acid sequence:Gly-Pro-Ala-Gly-Leu-Ala-Gly-Ala, which comprises a proteaserecognition site of a MMP-9 protease.
GPAGLAGA

SEQ ID NO: 162 - protein, 4 aa, synthetic construct
  REGION 1..4: note = SEQ ID NO: 162 is an amino acid sequence of an exemplary proteasesubstrate motif consisting of the amino acid sequence:Pro-Ala-Gly-Leu, which comprises a protease recognition site of aMMP-9 protease.
PAGL

SEQ ID NO: 163 - protein, 8 aa, synthetic construct
  REGION 1..8: note = SEQ ID NO: 163 is an amino acid sequence of an exemplary proteasesubstrate motif consisting of the amino acid sequence:Gly-Pro-Gly-Gly-Leu-Ala-Gly-Ala, which comprises a proteaserecognition site of a MMP-9 protease.
GPGGLAGA

SEQ ID NO: 164 - protein, 8 aa, synthetic construct
  REGION 1..8: note = SEQ ID NO: 164 is an amino acid sequence of an exemplary proteasesubstrate motif consisting of the amino acid sequence:Gly-Pro-Leu-Gly-Leu-Val-Gly-Gln, which comprises a proteaserecognition site of a MMP-1 protease.
GPLGLVGQ

SEQ ID NO: 165 - protein, 4 aa, synthetic construct
  REGION 1..4: note = SEQ ID NO: 165 is an amino acid sequence of an exemplary proteasesubstrate motif consisting of the amino acid sequence:Pro-Leu-Gly-Leu, which comprises a protease recognition site of aMMP-1 protease.
PLGL

SEQ ID NO: 166 - protein, 8 aa, synthetic construct
  REGION 1..8: note = SEQ ID NO: 166 is an amino acid sequence of an exemplary proteasesubstrate motif consisting of the amino acid sequence:Gly-Pro-Ala-Gly-Leu-Gly-Gly-Gly, which comprises a proteaserecognition site of a MMP-7 protease.
GPAGLGGG

SEQ ID NO: 167 - protein, 4 aa, synthetic construct
  REGION 1..4: note = SEQ ID NO: 167 is an amino acid sequence of an exemplary proteasesubstrate motif consisting of the amino acid sequence:Pro-Ala-Gly-Leu, which comprises a protease recognition site of aMMP-7 protease.
PAGL

SEQ ID NO: 168 - protein, 8 aa, synthetic construct
  REGION 1..8: note = SEQ ID NO: 168 is an amino acid sequence of an exemplary proteasesubstrate motif consisting of the amino acid sequence:Gly-Pro-Pro-Gly-Leu-Arg-Gly-Pro, which comprises a proteaserecognition site of a MMP-13 protease.
GPPGLRGP

SEQ ID NO: 169 - protein, 4 aa, synthetic construct
  REGION 1..4: note = SEQ ID NO: 169 is an amino acid sequence of an exemplary proteasesubstrate motif consisting of the amino acid sequence:Pro-Pro-Gly-Leu, which comprises a protease recognition site of aMMP-13 protease.
PPGL

SEQ ID NO: 170 - protein, 8 aa, synthetic construct
  REGION 1..8: note = SEQ ID NO: 170 is an amino acid sequence of an exemplary proteasesubstrate motif consisting of the amino acid sequence:Gly-Pro-Leu-Gly-Leu-Arg-Gly-Pro, which comprises a proteaserecognition site of a MMP-13 protease.
GPLGLRGP

SEQ ID NO: 171 - protein, 4 aa, synthetic construct
  REGION 1..4: note = SEQ ID NO: 171 is an amino acid sequence of an exemplary proteasesubstrate motif consisting of the amino acid sequence:Pro-Leu-Gly-Leu, which comprises a protease recognition site of aMMP-13 protease.
PLGL

SEQ ID NO: 172 - protein, 8 aa, synthetic construct
  REGION 1..8: note = SEQ ID NO: 172 is an amino acid sequence of an exemplary proteasesubstrate motif consisting of the amino acid sequence:Gly-Pro-Ala-Gly-Leu-Arg-Thr-Glu, which comprises a proteaserecognition site of a MMP-14 protease.
GPAGLRTE

SEQ ID NO: 173 - protein, 8 aa, synthetic construct
  REGION 1..8: note = SEQ ID NO: 173 is an amino acid sequence of an exemplary proteasesubstrate motif consisting of the amino acid sequence:Leu-Pro-Gln-Gly-Leu-Ala-Gly-Arg, which comprises a proteaserecognition site of a MMP-14 protease.
LPQGLAGR

SEQ ID NO: 174 - protein, 4 aa, synthetic construct
  REGION 1..4: note = SEQ ID NO: 174 is an amino acid sequence of an exemplary proteasesubstrate motif consisting of the amino acid sequence:Pro-Ala-Gly-Leu, which comprises a protease recognition site of aMMP-14 protease.
PAGL

SEQ ID NO: 175 - protein, 8 aa, synthetic construct
  REGION 1..8: note = SEQ ID NO: 175 is an amino acid sequence of an exemplary proteasesubstrate motif consisting of the amino acid sequence:Glu-Ala-Glu-Asn-Gly-Glu-Leu-Pro, which comprises a proteaserecognition site of a LGMN protease.
EAENGELP

SEQ ID NO: 176 - protein, 4 aa, synthetic construct
  REGION 1..4: note = SEQ ID NO: 176 is an amino acid sequence of an exemplary proteasesubstrate motif consisting of the amino acid sequence:Ala-Ala-Asn-Gly, which comprises a protease recognition site of aLGMN protease.
AANG

SEQ ID NO: 177 - protein, 5 aa, synthetic construct
  REGION 1..5: note = SEQ ID NO: 177 is an amino acid sequence of an exemplary proteasesubstrate motif consisting of the amino acid sequence:Asp-Asn-Phe-Leu-Val, which comprises a protease recognition siteof a Cathepsin A protease.
DNFLV

SEQ ID NO: 178 - protein, 5 aa, synthetic construct
  REGION 1..5: note = SEQ ID NO: 178 is an amino acid sequence of an exemplary proteasesubstrate motif consisting of the amino acid sequence:Asp-Asn-Phe-Phe-Val, which comprises a protease recognition siteof a Cathepsin A protease.
DNFFV

SEQ ID NO: 179 - protein, 8 aa, synthetic construct
  REGION 1..8: note = SEQ ID NO: 179 is an amino acid sequence of an exemplary proteasesubstrate motif consisting of the amino acid sequence:Gly-Leu-Ala-Gly-Gly-Ala-Gly-Gly, which comprises a proteaserecognition site of a Cathepsin B protease.
GLAGGAGG

SEQ ID NO: 180 - protein, 6 aa, synthetic construct
  REGION 1..6: note = SEQ ID NO: 180 is an amino acid sequence of an exemplary proteasesubstrate motif consisting of the amino acid sequence:Leu-Ala-Gly-Gly-Ala-Gly, which comprises a protease recognitionsite of a Cathepsin B protease.
LAGGAG

SEQ ID NO: 181 - protein, 9 aa, synthetic construct
  REGION 1..9: note = SEQ ID NO: 181 is an amino acid sequence of an exemplary proteasesubstrate motif consisting of the amino acid sequence:Gly-Leu-Val-Ala-Leu-Leu-Ala-Gly-Gly, which comprises a proteaserecognition site of a Cathepsin B protease.
GLVALLAGG

SEQ ID NO: 182 - protein, 6 aa, synthetic construct
  REGION 1..6: note = SEQ ID NO: 182 is an amino acid sequence of an exemplary proteasesubstrate motif consisting of the amino acid sequence:Leu-Glu-Val-Leu-Ile-Val, which comprises a protease recognitionsite of a Cathepsin D protease.
LEVLIV

SEQ ID NO: 183 - protein, 5 aa, synthetic construct
  REGION 1..5: note = SEQ ID NO: 183 is an amino acid sequence of an exemplary proteasesubstrate motif consisting of the amino acid sequence:Glu-Val-Leu-Ile-Val, which comprises a protease recognition siteof a Cathepsin D protease.
EVLIV

SEQ ID NO: 184 - protein, 8 aa, synthetic construct
  REGION 1..8: note = SEQ ID NO: 184 is an amino acid sequence of an exemplary proteasesubstrate motif consisting of the amino acid sequence:Glu-Val-Val-Leu-Val-Ala-Leu-Ala, which comprises a proteaserecognition site of a Cathepsin E protease.
EVVLVALA

SEQ ID NO: 185 - protein, 8 aa, synthetic construct
  REGION 1..8: note = SEQ ID NO: 185 is an amino acid sequence of an exemplary proteasesubstrate motif consisting of the amino acid sequence:Glu-Val-Val-Phe-Val-Ala-Leu-Ala, which comprises a proteaserecognition site of a Cathepsin E protease.
EVVFVALA

SEQ ID NO: 186 - protein, 4 aa, synthetic construct
  REGION 1..4: note = SEQ ID NO: 186 is an amino acid sequence of an exemplary proteasesubstrate motif consisting of the amino acid sequence:Val-Leu-Val-Ala, which comprises a protease recognition site of aCathepsin E protease.
VLVA

SEQ ID NO: 187 - protein, 4 aa, synthetic construct
  REGION 1..4: note = SEQ ID NO: 187 is an amino acid sequence of an exemplary proteasesubstrate motif consisting of the amino acid sequence:Val-Phe-Val-Ala, which comprises a protease recognition site of aCathepsin E protease.
VFVA

SEQ ID NO: 188 - protein, 8 aa, synthetic construct
  REGION 1..8: note = SEQ ID NO: 188 is an amino acid sequence of an exemplary proteasesubstrate motif consisting of the amino acid sequence:Asp-Val-Leu-Leu-Ser-Trp-Ala-Val, which comprises a proteaserecognition site of a Cathepsin G protease.
DVLLSWAV

SEQ ID NO: 189 - protein, 5 aa, synthetic construct
  REGION 1..5: note = SEQ ID NO: 189 is an amino acid sequence of an exemplary proteasesubstrate motif consisting of the amino acid sequence:Val-Leu-Leu-Ser-Trp, which comprises a protease recognition siteof a Cathepsin G protease.
VLLSW

SEQ ID NO: 190 - protein, 8 aa, synthetic construct
  REGION 1..8: note = SEQ ID NO: 190 is an amino acid sequence of an exemplary proteasesubstrate motif consisting of the amino acid sequence:Ala-Lys-Leu-Lys-Glu-Glu-Asp-Asp, which comprises a proteaserecognition site of a Cathepsin K protease.
AKLKEEDD

SEQ ID NO: 191 - protein, 8 aa, synthetic construct
  REGION 1..8: note = SEQ ID NO: 191 is an amino acid sequence of an exemplary proteasesubstrate motif consisting of the amino acid sequence:Ala-Gly-Leu-Gly-Glu-Glu-Asp-Asp, which comprises a proteaserecognition site of a Cathepsin K protease.
AGLGEEDD

SEQ ID NO: 192 - protein, 8 aa, synthetic construct
  REGION 1..8: note = SEQ ID NO: 192 is an amino acid sequence of an exemplary proteasesubstrate motif consisting of the amino acid sequence:Ala-Leu-Leu-Gly-Ala-Pro-Pro-Pro, which comprises a proteaserecognition site of a Cathepsin L protease.
ALLGAPPP

SEQ ID NO: 193 - protein, 8 aa, synthetic construct
  REGION 1..8: note = SEQ ID NO: 193 is an amino acid sequence of an exemplary proteasesubstrate motif consisting of the amino acid sequence:Gly-Leu-Leu-Gly-Ser-Glu-Pro-Glu, which comprises a proteaserecognition site of a Cathepsin L protease.
GLLGSEPE

SEQ ID NO: 194 - protein, 4 aa, synthetic construct
  REGION 1..4: note = SEQ ID NO: 194 is an amino acid sequence of an exemplary proteasesubstrate motif consisting of the amino acid sequence:Leu-Gly-Ala-Pro, which comprises a protease recognition site of aCathepsin L protease.
LGAP

SEQ ID NO: 195 - protein, 4 aa, synthetic construct
  REGION 1..4: note = SEQ ID NO: 195 is an amino acid sequence of an exemplary proteasesubstrate motif consisting of the amino acid sequence:Leu-Gly-Ser-Glu, which comprises a protease recognition site of aCathepsin L protease.
LGSE

SEQ ID NO: 196 - protein, 8 aa, synthetic construct
  REGION 1..8: note = SEQ ID NO: 196 is an amino acid sequence of an exemplary proteasesubstrate motif consisting of the amino acid sequence:Ala-Ala-Lys-Gly-Ala-Ala-Pro-Glu, which comprises a proteaserecognition site of a Cathepsin S protease.
AAKGAAPE

SEQ ID NO: 197 - protein, 4 aa, synthetic construct
  REGION 1..4: note = SEQ ID NO: 197 is an amino acid sequence of an exemplary proteasesubstrate motif consisting of the amino acid sequence:Leu-Gly-Ala-Ala, which comprises a protease recognition site of aCathepsin S protease.
LGAA

SEQ ID NO: 198 - protein, 8 aa, synthetic construct
  REGION 1..8: note = SEQ ID NO: 198 is an amino acid sequence of an exemplary proteasesubstrate motif consisting of the amino acid sequence:Ser-Ser-Gln-Tyr-Ser-Ser-Asn-Gly, which comprises a proteaserecognition site of a KLK3 protease.
SSQYSSNG

SEQ ID NO: 199 - protein, 8 aa, synthetic construct
  REGION 1..8: note = SEQ ID NO: 199 is an amino acid sequence of an exemplary proteasesubstrate motif consisting of the amino acid sequence:Ser-Gln-Gln-Tyr-Ser-Ser-Asn-Gly, which comprises a proteaserecognition site of a KLK3 protease.
SQQYSSNG

SEQ ID NO: 200 - protein, 8 aa, synthetic construct
  REGION 1..8: note = SEQ ID NO: 200 is an amino acid sequence of an exemplary proteasesubstrate motif consisting of the amino acid sequence:Ser-Ser-Gln-Gln-Ser-Ser-Asn-Gly, which comprises a proteaserecognition site of a KLK3 protease.
SSQQSSNG

SEQ ID NO: 201 - protein, 8 aa, synthetic construct
  REGION 1..8: note = SEQ ID NO: 201 is an amino acid sequence of an exemplary proteasesubstrate motif consisting of the amino acid sequence:Gly-Gly-Ser-Arg-Ser-Gly-Gly-Gly, which comprises a proteaserecognition site of a KLK2 protease.
GGSRSGGG

SEQ ID NO: 202 - protein, 8 aa, synthetic construct
  REGION 1..8: note = SEQ ID NO: 202 is an amino acid sequence of an exemplary proteasesubstrate motif consisting of the amino acid sequence:Gly-Gly-Ser-Arg-Ser-Pro-Gly-Gly, which comprises a proteaserecognition site of a KLK2 protease.
GGSRSPGG

SEQ ID NO: 203 - protein, 8 aa, synthetic construct
  REGION 1..8: note = SEQ ID NO: 203 is an amino acid sequence of an exemplary proteasesubstrate motif consisting of the amino acid sequence:Gly-Val-Asn-Leu-Asp-Val-Glu-Val, which comprises a proteaserecognition site of a beta-secretase 1 protease.
GVNLDVEV

SEQ ID NO: 204 - protein, 8 aa, synthetic construct
  REGION 1..8: note = SEQ ID NO: 204 is an amino acid sequence of an exemplary proteasesubstrate motif consisting of the amino acid sequence:Arg-Gln-Ala-Arg-Lys-Val-Gly-Gly, which comprises a proteaserecognition site of a matriptase-1 protease.
RQARKVGG

SEQ ID NO: 205 - protein, 8 aa, synthetic construct
  REGION 1..8: note = SEQ ID NO: 205 is an amino acid sequence of an exemplary proteasesubstrate motif consisting of the amino acid sequence:Ala-Ala-Ala-Arg-Lys-Val-Gly-Gly, which comprises a proteaserecognition site of a matriptase-1 protease.
AAARKVGG

SEQ ID NO: 206 - protein, 15 aa, synthetic construct
  REGION 1..15: note = SEQ ID NO: 206 is an amino acid sequence of protein 1 consistingof the amino acid sequence:Phe-Lys-Phe-Glu-Ala -Ala-Asn-Gly-Glu-Glu-Gly-Ser-Gly-Glu-Glu, inwhich the N-terminus is modified to comprise a Fmoc protecting
FKFEAANGEEGSGEE

SEQ ID NO: 207 - protein, 7 aa, synthetic construct
  REGION 1..7: note = SEQ ID NO: 207 is an amino acid sequence of protein 2 consistingof the amino acid sequence: Phe-Lys-Phe-Glu-Ala-Ala-Asn, in whichthe N-terminus is modified to comprise a Fmoc protecting group.
FKFEAAN

SEQ ID NO: 208 - protein, 18 aa, synthetic construct
  REGION 1..18: note = SEQ ID NO: 208 is an amino acid sequence of protein 3 consistingof the amino acid sequence:Phe-Lys-Phe-Glu-Leu -Ala-Gly-Gly-Ala-Gly-Glu-Glu-Gly-Ser-Gly-Glu-Glu-Glu, in which the N-terminus is modified to comprise a Fmoc
FKFELAGGAGEEGSGEEE